(19) United States
(12) Patent Application Publication
(10) Pub. No.: US 2026/0311332 A1
(43) Pub. Date: Oct. 8, 2026

(54) SYSTEMS AND METHODS FOR KARYOTYPING BY SEQUENCING

(71) Applicant: CytoTerra Inc., Seattle, WA (US)

(72) Inventors: Shawn SULLIVAN, Seattle, WA (US); Brad NELSON, Seattle, WA (US); Max PRESS, Seattle, WA (US); Zev KRONENBERG, Seattle, WA (US); Stephen EACKER, Seattle, WA (US); Ivan LIACHKO, Seattle, WA (US)

(73) Assignee: CytoTerra Inc., Seattle, WA (US)

(21) Appl. No.: 19/669,861

(22) Filed: May 6, 2026

Related U.S. Application Data
(63) Continuation of application No. 17/442,840, filed on Mar. 27, 2020.
(60) Provisional application No. 62/825,499, filed on Mar. 28, 2019.

(51) Int. Cl. G06T 7/00; C12Q 1/6806; C12Q 1/6869; G06T 5/70; G06T 7/13; G16B 20/00; G16B 40/00; G16B 40/20; G16B 45/00; G16H 10/40; G16H 10/60; G16H 50/20

(52) U.S. Cl. CPC G06T 7/0012; C12Q 1/6806; C12Q 1/6869; G06T 5/70; G06T 7/13; G16B 20/00; G16B 40/00; G16B 40/20; G16B 45/00; G16H 10/40; G16H 10/60; G16H 50/20; G06T 2207/30072; G06T 2207/30096

(57) ABSTRACT

The disclosure relates to methods and systems for identifying chromosomal structural variants in a subject using chromosomal conformational capture data, relating the chromosomal structural variants to diseases or disorders, and methods of treating same.

### CROSS-REFERENCES TO RELATED APPLICATIONS

This application is a continuation of U.S. application Ser. No. 17/442,840, filed on Sep. 24, 2021, which is a U.S. national phase of International Application No. PCT/US2020/025528, filed on Mar. 27, 2020, which claims the benefit and priority of U.S. Provisional Application No. 62/825,499, filed on Mar. 28, 2019, the disclosures of each of which are incorporated herein by reference in their entirety.

### BACKGROUND

For decades clinicians have used genetic tests to identify chromosomal structural variants, or genomic abnormalities, responsible for Mendelian diseases, cancers, autism and other human diseases. Similar tests are also employed for agricultural, veterinary, research and other purposes. The most common test to identify large-scale structural variation (SV) is karyotyping, whereby condensed metaphase chromosomes and visually inspected using various staining and microscopy techniques. A secondary, related technique that can confirm genomic rearrangements at specific loci is fluorescence in situ hybridization (FISH). Both karyotyping and FISH are labor intensive, time consuming, and require highly specialized training, limiting the throughput and efficiency of these methods. Furthermore, karyotyping methods are limited both by their resolution and by the need to obtain actively dividing cells, which can be difficult with liquid cancers such as blood and lymphatic cancers in clinical settings. There thus exists a need for additional methods accurately and rapidly identify chromosomal structural variants.

### SUMMARY

Systems and methods for identifying chromosomal structural variants using chromosomal conformational capture techniques, in any organism, tissue or cell type, are provided herein. In some embodiments of the systems and methods of the disclosure, the chromosomal structural variants are known and described in the art. In some alternative embodiments, the chromosomal structural variants are novel. The disclosure further provides systems and methods for relating chromosomal structural variants to biological information such as associated diseases or disorders, gene expression, and recommended treatments, and using this information to treat a disease or disorder in a subject.

Accordingly, the disclosure provides methods of treating a subject with a chromosomal structural variant comprising: (a) receiving a test set of reads from a sample from the subject; (b) aligning the test set of reads from the subject to a reference genome to produce a set of mapped reads from the subject; (c) training a machine learning model to distinguish between sets of reads from healthy subjects and sets of reads corresponding to known chromosomal structural variants; (d) applying the machine learning model to the mapped set of reads from the subject after training the machine learning model; (e) computing a likelihood that the subject has a known chromosomal structural variant based on applying the machine learning model to the mapped set of reads from the subject; and (f) generating a karyotype of the subject based on the likelihood the subject has the known chromosomal structural variant; wherein the test set of reads, the sets of reads from healthy subjects and the sets of reads corresponding to known chromosomal structural variants are generated by a chromosome conformation analysis technique. In some embodiments, the methods comprise generating geometric data structures from the test set of reads, the sets of reads from healthy subjects, and the sets of reads corresponding to known chromosomal structural variants.

In some embodiments of the methods of the disclosure, the methods comprise (a) receiving a test set of reads from a sample from the subject; (b) aligning the test set of reads from the subject to a reference genome to produce a mapped set of reads from the subject; (c) generating a geometric data structure from the mapped set of reads; (d) training a machine learning model to distinguish between geometric data structures from sets of reads from healthy subjects and sets of reads corresponding to known chromosomal structural variants; (e) applying the machine learning model to the geometric data structure from the subject after training the machine learning model; (f) computing a likelihood that the subject has a known chromosomal structural variant based on applying the machine learning model to the geometric data structure from the subject; and (g) generating a karyotype of the subject based on the likelihood the subject has the known chromosomal structural variant; wherein the test set of reads, the sets of reads from healthy subjects and the sets of reads corresponding to known chromosomal structural variants are generated by a chromosome conformation analysis technique.

In some embodiments of the methods of the disclosure, the known chromosomal structural variants each cause a disease or a disorder in a subject. In some embodiments, the methods further comprise treating the subject for the disease or disorder caused by the known chromosomal structural variant if the karyotype indicates that the subject has said known chromosomal structural variant.

In some embodiments of the methods of the disclosure, the chromatin conformation analysis technique comprises chromatin conformation capture (3C), circularized chromatin conformation capture (4C), carbon copy chromosome conformation capture (5C), chromatin immunoprecipitation (ChIP), ChIP-Loop, Hi-C, combined 3C-ChIP-cloning (6C), Capture-C, Split-pool barcoding (SPLiT-seq), Nuclear Ligation Assay (NLA), Single-cell Hi-C (scHi-C), Combinatorial Single-cell Hi-C, Concatamer Ligation Assay (COLA), Cleavage Under Targets and Release Using Nuclease (CUT& RUN), in vitro proximity ligation (e.g. Chicago®), in situ proximity ligation (in situ Hi-C), proximity ligation followed by sequencing on an Oxford Nanopore machine (Pore-C), proximity ligation sequenced on a Pacific Biosciences machine (SMRT-C), DNase Hi-C, Micro-C or Hybrid Capture Hi-C.

The disclosure provides systems for determining if a subject has a known chromosomal structural variant.

In some embodiments of the systems of the disclosure, the systems comprise: (a) a computer-readable storage medium which stores computer-executable instructions comprising: (i) instructions for receiving a test set of reads from a sample from the subject, wherein the test set of reads is generated by a chromosome conformation analysis technique; (ii) instructions for mapping the test set of reads from the subject onto a reference genome; (iii) instructions for applying a machine learning model to the test set of reads from the subject after training the machine learning model, wherein the machine learning model is trained to distinguish between sets of reads from healthy subjects and sets of reads corresponding to known chromosomal structural variants; (iv) instructions for computing a likelihood that the test set of reads contains a known chromosomal structural variant based on applying the machine learning model to the test set of reads; and (v) instructions for generating a karyotype of the subject based on the likelihood the subject has the known chromosomal structural variant; and (b) a processor which is configured to perform steps comprising: (i) receiving a set of input files which comprise the test set of reads from the subject and the reference genome; and (ii) executing the computer-executable instructions stored in the computer-readable storage medium.

In some embodiments of the systems of the disclosure, the systems comprise: (a) a computer-readable storage medium which stores computer-executable instructions comprising: (i) instructions for receiving a test set of reads from a sample from the subject, (wherein the test set of reads is generated by a chromosome conformation analysis technique; (ii) instructions for mapping the test set of reads from the subject onto a reference genome; (iii) instructions for generating a geometric data structure from the mapped set of reads; (iv) instructions for applying a machine learning model to the geometric data structure from test set of reads from the subject after training the machine learning model, wherein the machine learning model is trained to distinguish between geometric data structures sets of reads from healthy subjects and sets of reads corresponding to known chromosomal structural variants; (v) instructions for computing a likelihood that the geometric data structure from test set of reads contains a known chromosomal structural variant based on applying the machine learning model to the test set of reads; and (vi) instructions for generating a karyotype of the subject based on the likelihood the subject has the known chromosomal structural variant; and (b) a processor which is configured to perform steps comprising: (i) receiving a set of input files which comprise the test set of reads from the subject and the reference genome; and (ii) executing the computer-executable instructions stored in the computer-readable storage medium.

The disclosure provides methods of identifying chromosomal structural variants in a subject comprising: (a) training a first machine learning model to detect at least one region of a first contact matrix comprising at least one chromosomal structural variant; (b) receiving a first contact matrix from a subject by the first machine learning model, wherein the contact matrix is produced by a chromosome conformation analysis technique; (c) applying the first machine learning model to the first contact matrix to identify at least one region of the first contact matrix containing at least one chromosomal structural variant; (d) expressing each chromosomal structural variant identified by the first machine learning model as a bounding box comprising a start location and an end in a genome, and a label; (e) training a second machine learning model to relate the at least one chromosomal structural variant to biological information; (f) receiving the bounding box and the label of the at least one chromosomal structural variant identified by the first machine learning model by the second machine learning model; and (g) applying the second machine learning model, after training the second machine learning model; thereby identifying each chromosomal structural variant of the subject and the biological information related to each chromosomal structural variant.

The disclosure provides systems for identifying chromosomal structural variants in a subject comprising: (a) a computer-readable storage medium which stores computer-executable instructions comprising: (i) instructions for importing a first contact matrix from a subject into a first machine learning model, wherein the first contact matrix is produced by a chromosome conformation analysis technique; (ii) instructions for applying the first machine learning model to the contact matrix to detect at least one region of the first contact matrix comprising at least one chromosomal structural variant; (iii) instructions for expressing each chromosomal structural variant identified by the first machine learning model as a bounding box comprising a start and an end in a genome, and a label; (iv) instructions for receiving the bounding box and the label of the at least one chromosomal structural variant identified by the first machine learning model by a second machine learning model; and (v) instructions for applying the second machine learning model, wherein the second machine learning model is trained to relate a chromosomal structural variant to biological information, and wherein applying the second machine learning model occurs after training the second machine learning model; and (b) a processor which is configured to perform steps comprising: (i) receiving a set of input files which comprise at least the first contact matrix from the subject and the reference genome; and (ii) executing the computer-executable instructions stored in the computer-readable storage medium.

The disclosure provides methods of detecting chromosomal structural variants in a subject comprising: (a) receiving a contact matrix, wherein the contact matrix is produced by a chromosome conformation analysis technique applied to a sample from the subject; (b) representing the contact matrix as an image, wherein an intensity of each pixel in the image represents a density of links between two genomic locations in the contact matrix; and (c) applying image processing to the image; thereby detecting chromosomal structural variants in the subject.

The disclosure provides methods comprising: (a) contacting a sample from a subject with a stabilizing agent, wherein said sample comprises nucleic acids; (b) cleaving the nucleic acids into a plurality of fragments comprising at least a first segment and a second segment; (c) attaching the first segment and the second segment at a junction to generate a plurality of fragments comprising attached segments; (d) obtaining at least some sequence on each side of the junction of the plurality of fragments comprising attached segments to generate a plurality of reads; and (e) applying any of the machine learning models described herein.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a Hi-C proximity contact map showing the contact matrix of the first seven chromosomes from an acute myeloid leukemia (AML) sample. The dashed lines denote chromosome boundaries. Translocations appear as off-diagonal rectangular boxes between chromosome pairs one-five, two-six, and four-six.

FIG. 2 is a diagram showing an exemplary karyotyping by sequencing (KBS) embodiment of the disclosure. Left, a set of biological and/or clinical data, which may include variant, healthy, or simulated chromatin conformation data, as well as clinical or biological data about those samples or the organism(s) being analyzed, is used as input to train one or more models. Top, new clinical or research samples for which KBS analysis is desired are processed by a chromatin conformation capture protocol, which generates a chromatin conformation capture dataset after sequencing, alignment, and other processing. These data are provided as input to the trained models, which detect variants and their significance. Human-readable reports are finally generated from the analysis results.

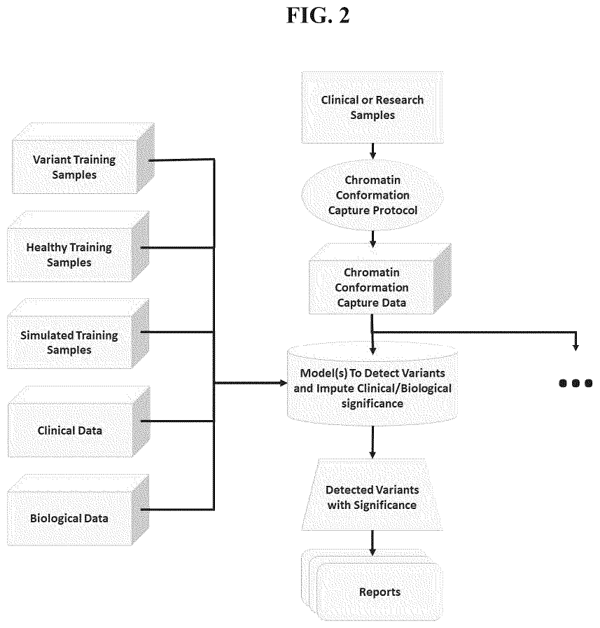

FIG. 3 is a block diagram that illustrates a variants identification system, according to an embodiment.

FIG. 4 is a diagram showing an exemplary karyotyping by sequencing embodiment of the disclosure, which can be used to genotype known structural variants in human samples. In box 402, healthy samples are processed with the Hi-C protocol and aligned to the human genome, resulting in a contact matrix. The contact matrices are used to train a negative binomial distribution (NBD) model. In box 404, a database containing variants of known clinical significance is manually curated. Variants are represented as genomic bands, similar to the nomenclature used in classical karyotyping. In box 406, new clinical or research samples are processed with the Hi-C protocol and aligned to the human genome, following the same methodology as in the training samples in box 402. The KBS variant detector uses the NBD model to calculate the likelihood that each known variant is present in the sample. All detected known variants are output by the KBS variant detector, including their significance from the clinical data. Human-readable reports similar to classical karyotype-based cytogenetics reports are generated.

FIG. 5 is a diagram showing an exemplary karyotyping by sequencing embodiment of the disclosure, which can be used for general purpose variant detection and annotation for any organism. In box 502, samples containing known variants, though not necessarily variants of known significance, are processed with Hi-C and aligned to the reference or draft genome, resulting in a contact matrix. Each variant in a sample is known, and used to label the type of variant. The contact matrixes from the samples are used at a mixture of resolutions to train a convolutional neural network (CNN) to detect the presence and type of variants in a sample. In box 504, data about samples containing structural variants of known clinical or biological significance are processed with the Hi-C protocol and aligned to the reference or draft assembly, resulting in a contact matrix. Clinical or biological data such as diagnoses, outcomes, drug/treatment response, metabolic effect, and other relevant data are used to train a k-nearest neighbors model (KNN) to associate contact matrix features with clinical or biological characteristics. In box 506, new clinical or research samples are processed with the Hi-C protocol and aligned to the reference or draft genome, following the same methodology as in the training samples in boxes 502 and 504. The KBS variant detector recursively uses the CNN, creating increasing resolution contact matrixes between classification steps, to precisely identify structural variants to the desired resolution. All detected known variants are then classified using the KNN model to predict the clinical and/or biological implications of the variant. Human-readable reports similar to classical karyotype-based cytogenetics reports are generated from the results.

FIG. 6 shows a contact matrix from a cancer sample that has been analyzed using the methods the disclosure. Corners are detected (Xs) within chr3 for a cancer sample. These corners correspond to structural variants detected on the chromosome. The units on the x- and y-axis are megabases.

FIG. 7 shows simulated Hi-C heat map data. Data was generated via introducing a synthetic structural variant mutation into the human genome and randomly generating proximity ligation interactions according to a statistical model reflecting the theoretical characteristics of the Hi-C protocol. The red rectangle off the main diagonal illustrates where this variant occurred, which was labeled as a translocation from chromosome 7 to chromosome 12 with a 0.98 confidence by the second major application.

FIG. 8 shows an exemplary visualization of a chromosomal conformational capture contact matrix as an image.

FIG. 9 shows the events detected by karyotyping by sequencing methods in a leukemia sample.

FIG. 10 is an image representing the processed matrix ready for use by the KBS Variant Detector. Raw Hi-C linkage densities are shown in the top right half of the matrix, and normalized Hi-C matrixes are shown on the bottom left half of the matrix. (A) Raw Hi-C linkage data show many details about genome architecture, such as the signature of a location from which an unbalanced translocation moved part of one copy of a chromosome. (B) Normalized Hi-C linkage data emphasize abnormal aspects of the dataset, such as interchromosomal translocations.

FIG. 11 is an image showing complex translocations create challenges for Hi-C-based structural variation callers. Zooming into the Hi-C matrix shows reciprocal translocations from chr2<->chr6 and chr4<->chr6 create an increased chr2: <->chr4 interaction signal.

### DETAILED DESCRIPTION

Computation methods and systems for the identification of chromosomal structural variants using chromatin conformation capture techniques are provided herein. In some embodiments, the disclosure further provides systems and methods for relating chromosomal structural variants to biological information pertinent to the chromosomal structural variant (for example, clinical data).

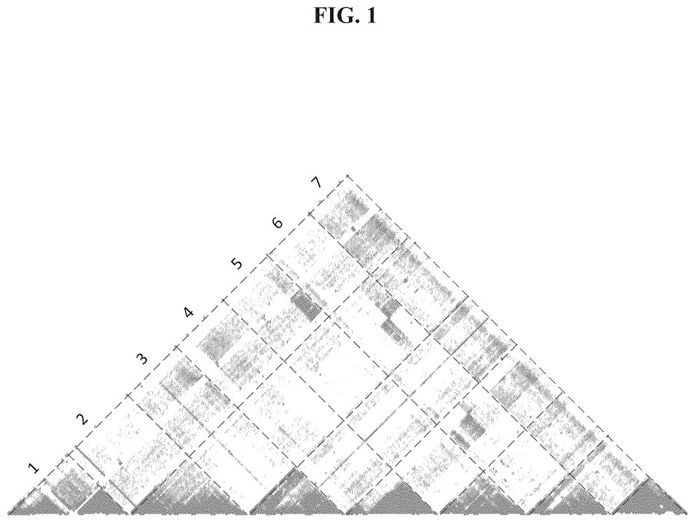

Chromatin conformation capture methods, such as 3-C, 4-C, 5-C, and Hi-C, physically link DNA molecules in close proximity inside intact cells. These methods measure how often two loci co-associate in space in vivo. A two-dimensional contact matrix is then calculated from chromatin conformation capture data by mapping high throughput sequencing reads from a chromatin conformation capture library to a draft or reference genome (FIG. 1). In a contact matrix, loci originating from the same chromosomes have a higher interaction frequency than loci on different chromosomes, and neighboring loci on the same chromosome have a higher interaction frequency than distal loci on that chromosome. Every individual's genome exhibits a slightly different contact matrix due to allelic variation within the individual's population of cells and mutations the individual was born with or acquired during their lifetime. These differences are termed variants. Some variants can be seen with the naked eye by visualizing the contact matrix as a contact map. Other variants can be detected by analyzing the contact matrix computationally. These variants include, but are not limited to, balanced and unbalanced translocations, inversions, and copy number variation such as insertions, deletions, repeat expansions, and other complex events. Some variants are known to have clinical significance, i.e. are associated with a disease and/or course of treatment. Other variants are of unknown clinical significance, or are novel (not previously described in the art). Chromatin conformation data and the methods and systems disclosed herein provide the means to describe variants of known clinical significance, and to discover variants of unknown clinical significance and novel variants.

Karyotyping by sequencing (KBS) methods of the disclosure use chromatin conformation data in clinical and research scenarios where karyotyping or karyotype-like data would be useful. This method includes multiple major applications. First, KBS methods are able to identify human genomic rearrangements observable by cytogenetic methods and to test for the presence of known clinically-reportable variants, in effect producing the same kind of actionable information as karyotyping but with highly different, powerful means. Second, KBS methods are capable of analyzing any sample to detect any structural variants, and classify these variants using any provided data about structural variation in the organism being sampled.

### Subjects

The disclosure provides methods and systems for identifying one or more chromosomal structural variants in a subject.

Subjects of the disclosure can be any organism. In some embodiments, the subject is a eukaryote. In some embodiments, the subject is a metazoan. In some embodiments, the subject is a vertebrate. In some embodiments, the subject is a mammal. In some embodiments, the subject is a human, a monkey, an ape, a rabbit, a guinea pig, a gerbil, a rat or a mouse. In some embodiments, the subject is an agricultural animal. Exemplary agricultural animals include horses, sheep, cows, pigs and chickens. In some embodiments, the subject is an animal that is kept as a pet (a veterinary subject). Exemplary pets include dogs and cats.

In some embodiments, the subject is a human.

In some embodiments, particularly those embodiments wherein the subject is a human, the subject has one or more symptoms of a disease or disorder which is caused by one or more chromosomal structural variants in the subject. In some embodiments, the chromosomal structural variant is one that is known in the art to cause a disease or disorder, or to affect the function of a gene or genes that cause a disease or disorder. In alternative embodiments, the chromosomal structural variant is a novel chromosomal structural variant, i.e. a variant that has not previously been described in the art. The disclosure provides systems and methods to identify both novel and known chromosomal structural variants.

The disclosure provides methods and systems for identifying one or more chromosomal structural variants in cells isolated or derived from any tissue or cell type in the subject. In some embodiments, the tissue is a healthy tissue of the subject, for example, healthy blood, skin, bone marrow, liver, kidney, neural tissue or muscle. In some embodiments, the tissue has one or more symptoms of a disease or disorder. In some embodiments, the disease or disorder is cancer, and the tissue comprises cancer cells. In some embodiments, the cancer comprises a solid tumor and the tissue comprises tumor cells. In some embodiments, the cancer comprises a liquid tumor, and tissue comprises white blood cells, blood progenitor cells, stem cells or bone marrow cells. In some embodiments, the tissue comprises a mixture of cells that comprise one or more chromosomal structural variants and cells that do not comprise one or more chromosomal structural variants.

As used herein “healthy subjects” do not have signs or symptoms of, or are not suspected of having, clinically significant chromosomal structural variants, or a disease caused by unknown structural variants. Chromosomal conformational sequencing information from samples from healthy subjects can be used, e.g., to train the machine learning models described herein, or for comparison purposes. Healthy subjects may be those whose genomes have been analyzed for CSVs by independent methods, such as conventional karyotyping or FISH. In some cases, healthy samples may contain CSVs, for example CSVs unrelated to a disease or disorder being analyzed using the methods described herein, or CSVs that are believed to have a minimal effect on the health of the subject.

“Healthy samples” include samples from healthy subjects. “Healthy samples” also include samples from subjects who have a disease or a disorder, but the healthy sample is from a tissue that is not affected by the disease or disorder. For example, if the subject has cancer, a test sample from a tumor of the cancer can be analyzed for chromosomal structural variants using the methods described herein, and compared to a healthy sample from a tissue from the same subject that does not have the tumor.

### Chromosomal Structural Variants

The disclosure provides methods and systems for identifying one or more chromosomal structural variants in a subject.

As used herein, the term “chromosome” refers to a chromatin complex comprising all or a portion of the genome of a cell. The genome of a cell is often characterized by its karyotype, which is the collection of all the chromosomes that comprise the genome of the cell. The genome of a cell can comprise one or more chromosomes. In humans, each chromosome has a short arm (termed “p” for “petit”) and a long arm (termed “q” for “queue”).

Each chromosome arm is divided into regions, or cytogenetic bands, that can be seen in a conventional karyotype using a microscope. The bands are labeled p1, p2, p3 etc. counting from the centromere out towards the telomeres. Higher-resolution sub-bands within the bands are sometimes also used to identify regions in the chromosome. Sub-bands are also numbered from the centromere out towards the telomere. Information on chromosome banding and chromosome nomenclature can be found in pp. 37-39 of Strachan, T. and Read, A. P. 1999. Human Molecular Genetics, 2nd ed. New York: John Wiley & Sons.

The terms “nucleic acid,” “polynucleotide,” and “oligonucleotide” are used interchangeably and refer to a deoxyribonucleotide or ribonucleotide polymer in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties. In general, an analogue of a particular nucleotide has the same base-pairing specificity (e.g., an analogue of A will base pair with T. A polynucleotide of deoxyribonucleic acids (DNA) of specific identities and order is also referred to herein as a “DNA sequence.” Chromosomes comprise polynucleotides complexed with proteins (e.g. histones).

As used herein the terms “Structural Variant”, “Chromosomal Structural Variant”, “CSV” or “SV” refer to a difference in the structure of an individual's chromosome or chromosomes relative to the chromosome(s) in the genomes of other individuals within the same species or in a closely related species. Differences in chromosomal structure encompass differences in the arrangement and identity of DNA sequences in a chromosome. Differences in the arrangement of DNA sequences in a chromosome include both differences in the positions of DNA sequences on the chromosome relative to other sequences (e.g., translocations) and differences in orientation relative to other sequences (e.g., inversions). Differences in the identity of DNA sequences along a chromosome can include both new sequences or missing sequences, for example through the movement sequences from one chromosome to another non-homologous chromosome.

Chromosomal structural variations can be small or large in size, encompassing tens of base pairs, hundreds of base pairs, kilobases, megabases, or even significant portions (a half, a third or three-quarters, e.g.) of an individual chromosome. All size of chromosomal structural variations are within the scope of the disclosure.

There are multiple types of chromosomal structural variants, all of which are envisaged as within the scope of the methods and systems of the disclosure. Non-limiting examples of types of chromosomal structural variants include a translocation, a balanced translocation, an unbalanced translocation, a complex translocation, an inversion, a deletion, a duplication, a repeat expansion or a ring.

As used herein the term “translocation” refers to the exchange of DNA sequences between non-homologous chromatids, between two or more positions on the same chromatid, or between homologous chromatids that is not as a result of crossover during meiosis. Translocations can create gene fusions, which occur when two genes that are not normally adjacent to each other are brought into proximity. Alternatively, or in addition, translocations can disrupt gene function by breaking genes at the borders of the translocation. For example, a translocation can separate an open reading frame (ORF) from a distal regulatory element or bring the open reading frame into proximity with a new regulatory element, thereby affecting gene expression. Alternatively, or in addition, the break point of the translocation can occur in the middle of a gene, thereby creating a gene truncation. A “breakpoint” refers to the point or region of a chromosome at which the chromosome is cleaved during a translocation. A “breakpoint junction” refers to the region of the chromosome at which the different parts of chromosomes involved in a translocation join. Alternatively, or in addition, a translocation can affect the expression of one or more genes contained within the translocation by moving those genes to a new chromatin environment in the nucleus, for example by moving a DNA sequence from a region of strong gene expression (e.g. euchromatin) to a region of low gene expression (e.g. heterochromatin) or vice versa. Depending on the translocation, the translocation can have no effect on gene expression, can effect a single gene, or can effect multiple genes.

As used herein the term “balanced translocation” refers to the reciprocal exchange of DNA between non-homologous chromatids, or between homologous chromatids not as a result of crossover during meiosis. A “balanced translocation” is a translocation in which there is no loss of genetic material during the translocation, but all genetic material is preserved during the exchange. In an “unbalanced translocation” there is a loss of genetic material during the exchange.

As used herein, the term “reciprocal translocation” refers to a translocation which involves the mutual exchange of fragments between two broken chromosomes. In a reciprocal translocation, one part of one chromosome unites with the part of another chromosome.

As used herein, the terms “variant translocation”, “abnormal translocation” or “complex translocation” refer to the involvement of a third chromosome in a secondary rearrangement that follows a first translocation.

Translocations can be intrachromosomal (the rearrangement breakpoints occur within the same chromosome) or interchromosomal (the rearrangement breakpoints are between two different chromosomes).

As used herein, the term “inversion” refers to the rearrangement of DNA sequences within the same chromosome. Inversions change the orientation of a DNA sequence within a chromosome.

As used herein, the term “deletion” refers to a loss of a DNA sequence. Deletions can be any size, ranging from a few nucleotides to entire chromosomes. Translocations are frequently accompanied by deletions, for example at the translocation break points.

As used herein, the term “duplication” refers to a duplication of a DNA sequence (e.g., the genome contains three copies of a DNA sequence, instead of two). Duplications can be any size, ranging from a few nucleotides to entire chromosomes. Translocations are frequently accompanied by duplications.

As used herein, the term “repeat expansion” refers to tandem repeated sequences in the genome that with variable copy numbers between subjects. When there are a greater than average number of repeats of a repetitive sequence, the repetitive sequence has been expanded. Repeated sequences can comprise 2, 3, 4, 5, 6, 7, 8, 9, 10 or more repeated nucleotides. Expanded repeats are associated with a number of genetic disorders, including but not limited to Huntington's disease, spinocerebellar ataxias, fragile X syndrome, myotonic dystrophy, Friedreich's ataxia and juvenile myoclonic epilepsy.

All types of chromosomal structural variants can be identified using the methods and systems of the disclosure.

In some embodiments, the chromosomal structural variant identified by the methods and systems of the disclosure is a chromosomal variant that is known in the art. For example, the chromosomal structural variant identified by the methods of the disclosure is a chromosomal structural variant that has been previously described and characterized. Descriptions of chromosomal structural variants in the art include mapping one or more breakpoints of the chromosomal structural variant using techniques known in the art, for example by karyotyping, sequencing or Southern blot. In those embodiments wherein the chromosomal structural variant is known to cause a disease or disorder, descriptions of known chromosomal structural variants include clinical data such as symptoms, prognosis and recommended courses of treatment.

In some embodiments, the chromosomal structural variant identified by the methods and systems of the disclosure is a novel chromosomal variant. Novel chromosomal structural variants are variants that have not previously been described in the art. Novel chromosomal structural variants may be similar to chromosomal structural variants known in the art. For example, a chromosomal structural variant may be both recurrent, in that similar variants occur independently across multiple individuals, and novel, in that each individual with a recurrent variant comprises a variant with slightly different break points. In some embodiments, a novel chromosomal structural variant has one or more breakpoints that are similarly placed compared to a break point of a chromosomal structural variant known in the art. A similarly placed break point comprises a break point that is within 50 bp, within 100 bp, within 500 bp, within 1 kb, within 5 kb, within 10 kb, within 20 kb, within 50 kb, within 100 kb, within 200 kb or within 500 kb or within 1 Mb of a break point of a chromosomal structural variant known in the art. In some embodiments, a novel chromosomal structural variant has one or more breakpoints that are identical to a break point of a chromosomal structural variant known in the art, and one or more breakpoints that are not identical to a break point of a chromosomal structural variant known in the art. In some embodiments, a novel chromosomal structural variant does not have similar or identical break points to a chromosomal structural variant known in the art.

### Representation of Chromosomal Structural Variants

The disclosure provides systems and methods for identifying one or more chromosomal structural variants in a subject, and representing the chromosomal structural variant or variants in a manner that can be readily interpreted by a person of ordinary skill in the art (for example, a clinician, a doctor, a patient or a researcher).

In some embodiments, the chromosomal structural variant is represented as a karyotype. Karyotyping is a traditional method used to identify chromosomal structural variants. In karyotyping, the development of cells is arrested during metaphase, bound chromatids are extracted, stained and photographed, and the structural properties of the chromatids are mapped using the cytogenetic banding patterns of the chromosome. Karyotyping is expensive, time consuming and of limited resolution. Traditional karyotyping relies on the cytogenetic bands and sub bands within the karyotype to map the boundaries of chromosomal structural variants, and so cannot resolve chromosomal structural variants that are finer (smaller) than the cytogenetic bands of the karyotype, which typically have a minimum resolution of about 5 Mb. In contrast, the systems and methods of the disclosure are able to achieve a resolution that is at least 1,000 finer than a traditional karyotype.

One method used in karyotyping is Flow cytometry (FC) and fluorescence in situ hybridization (FISH) which can be used to detect aneuploidy in any phase of the cell cycle. FISH is used identify the physical location of specific DNA sequences on a chromatid using fluorescent probes. FISH probes are short DNA oligos linked to fluorophores. FISH probes, once hybridized, can be visualized using optical microscopy accompanied by fluorophore excitation. When two or more FISH probes, with different fluorophore colors, are used the coarse distance and orientation between two loci can be estimated. One advantage of this method is that it is less expensive than karyotyping, but the cost is still significant enough that generally only a small selection of chromosomes are tested (for humans, usually chromosomes 13, 18, 21, X, Y; also sometimes 8, 9, 15, 16, 17, 22). In contrast, the systems and methods of the disclosure can rapidly and cheaply karyotype all chromosomes in a subject. In addition, FISH has a low level of specificity. Using FISH to analyze 15 cells, one can detect mosaicism of 19% with 95% confidence. The reliability of the test becomes much lower as the level of mosaicism gets lower, and as the number of cells to analyze decreases. The test is estimated to have a false negative rate as high as 15% when a single cell is analyzed. Thus, there is a great demand for a method that has a higher throughput, lower cost, and greater accuracy, such as the methods provided herein.

Traditional karyotype results can be represented as karyotype spreads, which are images of all the chromosomes analyzed in the karyotype, stained to identify cytogenetic bands and arranged in ordered pairs. While the methods of the disclosure provide a resolution superior to a traditional karyotype, the chromosomal structural variants identified by the methods of the disclosure can be represented as a karyotype or karyotype spread. This facilitates interpretation of chromosomal structural variant data of the disclosure by doctors and clinicians, who may be more familiar with and trained to identify chromosomal structural variants based on traditional karyotypes.

In some embodiments, chromosomal structural variants of the disclosure are represented as a karyotype.

In some embodiments, chromosomal structural variants identified by the methods and systems of the disclosure are represented as a bounding rectangle. In some embodiments, the bounding rectangle comprises a start location and end location in the genome of the chromosomal structural variant, and a label.

In some embodiments, chromosomal structural variants identified by the methods and systems of the disclosure are represented as a genomic coordinates and a label.

In some embodiments, the label comprises the type of chromosomal structural variant identified by the methods and systems of the disclosure. For example, the label identifies the chromosomal structural variant as a translocation, a balanced translocation, an inversion, a deletion, a duplication or a ring.

In some embodiments, the label identifies biological information relevant to the chromosomal structural variant identified by the methods and systems of the disclosure. For example, the label indicates what diseases or disorders are associated with the chromosomal structural variant, what genes are affected, and/or a course of treatment.

In some embodiments, the label comprises the genomic coordinates of a chromosomal structural variant identified by the systems and methods of the disclosure.

In some embodiments, the label comprises information about the chromosomal structural variant that has been created by a first machine learning model that is used as an input for a second machine learning model. For example, a first machine learning machine learning model identifies and labels one or more chromosomal structural variants, and a second machine learning machine learning model relates the identified chromosomal structural variant(s) to relevant biological information. In some embodiments, the first machine learning machine learning model is a likelihood classifier that uses a convolutional neural network trained to identify chromosomal structural variants from chromosomal conformational capture data. In some embodiments, the second machine learning model is a recurrent neural network or a sense detector that is trained is trained using clinical label data from known chromosomal structural variations.

### Clinical Chromosomal Structural Variants

The disclosure provides methods and systems for identifying one or more chromosomal structural variants in a subject, and further relating the one or more chromosomal structural variants to relevant biological information. Relevant biological information includes, but is not limited to, the clinical significance of the variant, associated diseases or disorders, symptoms thereof, associated genes and/or genetic mutations, effects of the chromosomal structural variant on gene expression, and recommended courses of treatment or therapies.

In some embodiments, the chromosomal structural variants that are identified by the systems and methods of the disclosure cause one or more diseases or disorders.

In some embodiments, the chromosomal structural variants that cause diseases or disorders are inherited, i.e. the chromosomal structural variant is transmitted from parent to offspring via the germ line. All inherited chromosomal structural variants are within the scope of the systems and methods of the disclosure.

In other alternative embodiments, the chromosomal structural variants that cause diseases or disorders are somatic, i.e. the chromosomal structural variant arise de novo in a cell in the individual. Depending upon when in development a somatic chromosomal structural variant arises, somatic chromosomal structural variants can occur all the cells in an organism (the chromosomal structural variant arises prior to the first cell division), or can occur in a subset of the cells in the organism (the chromosomal structural variant occurs later in development, or in an adult). Exemplary disorders that can occur in every cell include aneuploidies such as Turner syndrome (X chromosome monosomy) and Down syndrome (trisomy 21).

Exemplary disorders caused by haploinsufficiencies resulting from deletions include Williams syndrome, Langer-Giedion syndrome, Miller-Dieker syndrome, and DiGeorge/velocardiofacial syndrome. All somatic chromosomal structural variants are within the scope of the systems and methods of the disclosure.

In some embodiments, the diseases or disorders caused by chromosomal structural variants are caused by a chromosomal structural variant that occurs de novo in the subject. In some embodiments, the chromosomal structural variant that occurs de novo is a recurrent structural variant. Many chromosomal structural variants are recurrent, in that the same or similar chromosomal structural variants occur de novo in multiple individuals. These individuals are not necessarily related. In many cases, the recurrent chromosomal structural variants are caused by non-allelic homologous recombination mediated by flanking segmental duplications. In non-allelic homologous recombination, improper crossing over between non-homologous DNA sequences, for example DNA sequences that contain similar repetitive DNA sequences, leads to a tandem or direct duplication and a deletion. Non-limiting examples of diseases and disorders caused by recurrent chromosomal structural variants include in Charcot Marie Tooth disease, hereditary neuropathy with liability to pressure palsies, Prader Willi, Angelman, Smith Magenis, DiGeorge/velocardiofacial (DGS/VCFS), Williams Beurens, and Sotos syndromes.

Databases of chromosomal structural variants are known to persons of ordinary skill in the art. For example, biological information regarding chromosomal structural variants and their associated diseases and disorders, and treatments for these diseases and disorders can be found in the Online Mendelian Inheritance in Man (www.omim.org), the Mitelman Database of Chromosome Aberration and Gene Fusion in Cancer (cgap.nci.nih.gov/Chromosomes/Mitelman) and the NCBI database (www.ncbi.nlm.nih.gov/clinvar?term=300005 [MIM]).

Exemplary diseases and disorders associated with chromosomal structural variants are shown in table 1.

TABLE 1
Diseases and genes associated with chromosomal structural variants

| Title | Cytogenetic Location | Genomic Coordinates (GRCh38) |
|---|---|---|
| Huntington disease | 4p16.3 | |
| Hemoglobin H disease | 16p13.3, 16p13.3 | |
| Alzheimer's disease | 21q21.3 | 21: 25880549-26171127 |
| heart defects, congenital, and other | 18q11.2 | |
| congenital anomalies | | |
| myeloproliferative disease, | | |
| autosomal recessive | | |
| adrenal hyperplasia, congenital, due | 6p21.33 | |
| to 21-hydroxylase deficiency | | |
| macular dystrophy, vitelliform, 2 | 11q12.3 | |
| dupuytren contracture | 16q11.1-q22 | 16: 36800000-74100000 |
| holoprosencephaly 1 | 21q22.3 | 21: 41200000-46709983 |
| chromosome 18q deletion syndrome | 18q | 18: 18500000-80373285 |
| corneal dystrophy, fuchs endothelial, | 1p34.3 | |
| 1 | | |
| Rett syndrome (mecp2) | Xq28 | X: 154021799-154097730 |
| schizophrenia | 1p36.2, 1p36.22, | |
| | 1q32.1, 1q42.2, | |
| | 3p25.2, 3q13.31, | |
| | 5q23-q35, 6p23, | |
| | 6q13-q26, 8p21, | |
| | 10q22.3, 11q14-q21, | |
| | 13q14.2, 13q32, | |
| | 13q33.2, 14q32.33, | |
| | 18p, 22q11.21, | |
| | 22q11.21, 22q12.3, | |
| | 22q12.3 | |
| Friedreich ataxia 1 | 9q21.11 | |
| incontinentia pigmenti | Xq28 | |
| retinitis pigmentosa (rpgr) | Xp11.4 | X: 38269162-38327541 |
| macular dystrophy, retinal, 1, north | 6q16.2 | |
| carolina type (mcdr1) | | |
| 21-hydroxylase deficiency (cyp21a2) | 6p21.33 | 6: 32038315-32041669 |
| premature ovarian failure 1; pof1 | Xq27.3 | |
| interstitial lung disease, dyskeratosis | 20q13.33 | 20: 63657809-63696252 |
| congenita and hoerall-hreidarsson | | |
| syndrome (rtel1) | | |
| tetralogy of Fallot; tof | 5q35.1, 8p23.1, | |
| | 8q23.1, 18q11.2, | |
| | 20p12.2, 22q11.21 | |
| Alzheimer's disease (uchl1) | 4p13 | 4: 41256880-41268428 |
| Digeorge syndrome 2, | 10p14 | 10: 8045419-8075200 |
| hypoparathyroidism deafness and | | |
| renal dysplasia syndrome (gata3) | | |
| mucopolysaccharidosis type vii | 7q11.21 | 7: 65960683-65982313 |
| (beta-glucuronidase; gusb) | | |
| blepharophimosis, ptosis, and | 3q22.3 | |
| epicanthus inversus; bpes | | |
| systemic lupus erythematosis (fc | 1q23.3 | 1: 161647242-161678653 |
| fragment of igg, low affinity iib, | | |
| receptor for; fcgr2b) | | |
| albinism, oculocutaneous, type ia; | 11q14.3 | |
| oca1a | | |
| c syndrome | 3q13.1-q13.2 | |
| diaphragmatic hernia, congenital | 15q26.1 | 15: 88500000-93800000 |
| macrocephaly/megalencephaly | 9p23-p22 | 9: 14081842-14398982 |
| (nuclear factor i/b; nfib) | | |
| superoxide dismutase 2; sod2 | 6q25.3 | 6: 159679063-159762528 |
| mucopolysaccharidosis, type iiia; | | |
| mps3a | 17q25.3 | |
| Meckel syndrome, type 1; mks1 | 17q22 | |
| Angelman syndrome (ubiquitin- | | |
| protein ligase e3a; ube3a) | 15q11.2 | 15: 25337233-25439380 |
| mucopolysaccharidosis, type ii; | | |
| mps2 | Xq28 | |
| Noonan syndrome 1; ns1 | 12q24.13 | |
| fragile x syndrome; fxs | Xq27.3 | |
| small nucleolar rna host gene 14; | 15q11.2 | 15: 24823607-25419461 |
| snhg14 | | |
| autism | 7q22 | 7: 98400000-107800000 |
| cat eye syndrome; ces | 22q11 | 22: 15000000-25500000 |
| chronic lymphocytic and heavy | 14q32.33 | 14: 105741472-105743069 |
| chain deposition disease (igg heavy | | |
| chain locus; ighg1) | | |
| keratin 10, type i; krt10 | 17q21.2 | 17: 40818116-40822620 |
| preeclampsia/eclampsia 1; pee1 | 2p13 | 2: 68400000-74800000 |
| x-linked alport syndrome (collagen, | Xq22.3 | X: 108439843-108697544 |
| type iv, alpha-5; c014a5) | | |
| aprataxin; aptx | 9p21.1 | 9: 32883871-33025130 |
| Gilles de la Tourette syndrome; gts | 11q23 | 11: 110600000-121300000 |
| epilepsy (cholinergic receptor, | 15q13.3 | 15: 32030461-32172520 |
| neuronal nicotinic, alpha polypeptide | | |
| 7; chrna7) | | |
| hypomelanosis of ito; hmi | | |
| choroideremia; chm | Xq21.2 | |
| danubian endemic familial | | |
| nephropathy | | |
| aceruloplasminemia | 3q24-q25 | |
| renal tubular acidosis (solute carrier | 17q21.31 | 17: 44248389-44268160 |
| family 4 (anion exchanger), member | | |
| 1; slc4a1) | | |
| galactosemia | 9p13.3 | |
| insensitivity to pain, thyroid disease | 1q23.1 | 1: 156815749-156881849 |
| (neurotrophic tyrosine kinase, | | |
| receptor, type 1; ntrkl) | | |
| mandibulacral displasia (zinc | 1p34.2 | 1: 40258049-40294183 |
| metalloproteinase ste24; zmp5te24) | | |
| thrombocytopenia-absent radius | 1q21.1 | |
| syndrome; tar | | |
| osteogenesis imperfecta, type ii; oi2 | 7q21.3, 17q21.33 | |
| dyskeratosis congenita, autosomal | 20q13.33 | |
| recessive 5; dkcb5 | | |
| Ellis-van Creveld syndrome; eve | 4p16.2, 4p16.2 | |
| immunodeficiency 41 with | 10p15.1 | |
| lymphoproliferation and | | |
| autoimmunity; imd41 | | |
| congenital anomalies of kidney and | 1q23.3 | |
| urinary tract syndrome with or | | |
| without hearing loss, abnormal ears, | | |
| or developmental delay; cakuthed | | |
| phosphoglycerate kinase deficiency | Xq21.1 | X: 78104168-78126826 |
| (phosphoglycerate kinase 1; pgkl) | | |
| Axenfeld-Rieger syndrome, type 1; | 4q25 | |
| riegl | | |
| campomelic dysplasia | 17q24.3 | |
| Hermansky-Pudlak syndrome 2; | 5q14.1 | |
| hps2 | | |
| microcephaly 5, primary, autosomal | 1q31.3 | |
| recessive; mcph5 | | |
| immunodeficiency, common | 2q33.2 | |
| variable, 1; cvid1 | | |
| corpus callosum, agenesis of, with | | |
| facial anomalies and robin sequence | | |
| gout (urate oxidase, pseudogene; | 1p22 | 1: 84400000-94300000 |
| uox) | | |
| tetralogy of fallot (paired-like | | |
| homeodomain transcription factor 2; | | |
| pitx2) | 4q25 | 4: 110617422-110642122 |
| Fanconi anemia (fancc gene; fancc) | 9q22.32 | 9: 95099053-95317729 |
| osteochondrodysplasia | 4p15.32 | 4: 16160504-16226537 |
| (transmembrane anterior posterior | | |
| transformation 1; taptl) | | |
| Holt-Oram syndrome; hos | 12q24.21 | |
| severe combined immunodeficiency, | | |
| autosomal recessive, t cell-negative, | 20q13.12 | |
| b cell-negative, nk cell-negative, due | | |
| to adenosine deaminase deficiency | | |
| peroxisome biogenesis disorders | 7q21.2 | 7: 92487022-92528530 |
| (peroxisome biogenesis factor 1; | | |
| pexl) | | |
| trichorhinophalangeal syndrome, | 8q23.3 | |
| type i; trpsl | | |
| chromosome 15q13.3 deletion | 15q13.3 | 15: 30900000-33400000 |
| syndrome | | |
| folate deficiency (dihydrofolate | 5q14.1 | 5: 80626225-80654980 |
| reductase; dhfr) | | |
| immunoglobulin kappa light chain | 2p11.2 | 2: 88857360-88857682 |
| deficiency, deposition disease | | |
| (immunoglobulin kappa light chain | | |
| constant region; igkc) | | |
| fg syndrome 4 (calcium/calmodulin- | Xp11.4 | X: 41514933-41923524 |
| dependent serine protein kinase; | | |
| cask) | | |
| chromosome xq28 duplication | Xq28 | X: 148000000-156040895 |
| syndrome | | |
| omphalocele, autosomal | 1p31.3 | 1: 60800000-68500000 |
| t-cell immunodeficiency, recurrent | 20q13.12 | |
| infections, and autoimmunity with or | | |
| without cardiac malformations; tiiac | | |
| chromosome 14q11-q22 deletion | 14q11-q22 | 14: 17200000-57600000 |
| syndrome | | |
| ring chromosome 14 syndrome | Chr.14 | |
| Dandy-Walker syndrome; dws | 3q22-q24 | 3: 129500000-149200000 |
| blood group, xg system; xg | Xpter-p22.32 | |
| osteogenesis imperfecta (collagen, | 7q21.3 | 7: 94394560-94431231 |
| type i, alpha-2; c011a2) | | |
| liver disease (haptoglobin; hp) | 16q22.2 | 16: 72054591-72061055 |
| skeletal malformation | 2q31.1 | |
| (brachydactyly, type el; bdel) | | |
| cone-rod dystrophy 17; cord17 | 10q26 | 10: 117300000-133797422 |
| spastoc paraplegia (wd repeat- | 3p22.2 | 3: 39051985-39096670 |
| containing protein 48; wdr48) | | |
| catechol-o-methyltransferase; comt | 22q11.21 | 22: 19941739-19969974 |
| kidney disease (complement factor | 1q31.3 | 1: 196975021-197009724 |
| h-related 5; cfhr5) | | |
| clotting diseases (coagulation factor | 11p11.2 | 11: 46719165-46739507 |
| ii; f2) | | |
| Hunter syndrome (iduronate 2- | Xq28 | X: 149476989-149505353 |
| sulfatase; ids) | | |
| spondylocostal dysostosis 5; scdo5 | 16p11.2 | |
| aniridia 2; an2 | 11p13 | |
| peroxisome biogenesis disorders | 6p21.1 | 6: 42963872-42980223 |
| (peroxisome biogenesis factor 6; | | |
| pex6) | | |
| Hermansky-Pudlak syndrome type 2 | 5q14.1 | 5: 78002325-78294754 |
| (adaptor-related protein complex 3, | | |
| beta-1 subunit; ap3b1) | | |
| chromosome 15q11-q13 duplication | 15q11 | 15: 19000000-25500000 |
| syndrome | | |
| Kallmann syndrome (kal1 gene; | Xp22.31 | X: 8528873-8732186 |
| kal1) | | |
| cardiomyopathy, ovarian disorders | 20p12.3 | 20: 5950651-6000940 |
| (minichromosome maintenance | | |
| complex component 8; mcm8) | | |
| Waardenburg syndrome (paired box | 2q36.1 | 2: 222199886-222298995 |
| gene 3; pax3) | | |
| immunodeficiency, inflammatory | 5p13.2 | 5: 35856848-35879602 |
| diseases (interleukin 7 receptor; i17r) | | |
| sc phocomelia syndrome | 8p21.1 | |
| clotting disorders (coagulation factor | 5q35.3 | 5: 177402137-177409575 |
| xii; f12) | | |
| microcephaly, seizure (valyl-trna | 6p21.33 | 6: 31777517-31795934 |
| synthetase; vars) | | |
| albinism (leucine-rich melanocyte | 10q22.2-q22.3 | 10: 75431645-76557374 |
| differentiation-associated protein; | | |
| lrmda) | | |

Chromosomal structural variants and associated diseases and disorders are also described by the National Institute of Health's Genetic and Rare Diseases Information Center (rarediseases.info.nih.gov/diseases/diseases-by-category/36/chromosome-disorders). Chromosomal structural variants with clinical significance include, but are not limited to, 15q13.3 microdeletion syndrome, 16p11.2 deletion syndrome, 17q23.1q23.2 microdeletion syndrome, 1q duplications, 1q21.1 microdeletion syndrome, 22q11.2 deletion syndrome, 22q11.2 duplication syndrome, 2q23.1 microdeletion syndrome, 2q37 deletion syndrome, 47 XXX syndrome, 47, XYY syndrome, 49,XXXXX syndrome, Cat eye syndrome, Chromosome 1, uniparental disomy 1q12 q21, Chromosome 10p deletion, Chromosome 10p duplication, Chromosome 10q deletion, Chromosome 10q duplication, Chromosome 11p deletion, Chromosome 11p duplication, Chromosome 11q deletion, Chromosome 11q duplication, Chromosome 12p deletion, Chromosome 12p duplication, Chromosome 12q deletion, Chromosome 12q duplication, Chromosome 13q deletion, Chromosome 13q duplication, Chromosome 14q deletion, Chromosome 14q duplication, Chromosome 15q deletion, Chromosome 15q duplication, Chromosome 16 trisomy, Chromosome 16p deletion, Chromosome 16p duplication, Chromosome 16q deletion, Chromosome 17p deletion, Chromosome 17p duplication, Chromosome 17q duplication, Chromosome 18p deletion, Chromosome 18p tetrasomy, Chromosome 19p deletion, Chromosome 19p duplication, Chromosome 19q deletion, Chromosome 19q duplication, Chromosome 1p deletion, Chromosome 1p duplication, Chromosome 1p36 deletion syndrome, Chromosome 1q deletion, Chromosome 1q21.1 duplication syndrome, Chromosome 20 trisomy, Chromosome 20p deletion, Chromosome 20p duplication, Chromosome 20q deletion, Chromosome 20q duplication, Chromosome 21q deletion, Chromosome 21q duplication, Chromosome 22q deletion, Chromosome 2p deletion, Chromosome 2p duplication, Chromosome 2q deletion, Chromosome 2q duplication, Chromosome 2q24 microdeletion syndrome, Chromosome 3p deletion, Chromosome 3p duplication, Chromosome 3p-syndrome, Chromosome 3q deletion, Chromosome 3q duplication, Chromosome 3q29 microduplication syndrome, Chromosome 4p deletion, Chromosome 4p duplication, Chromosome 4q deletion, Chromosome 4q duplication, Chromosome 5p deletion, Chromosome 5p duplication, Chromosome 5q deletion, Chromosome 5q duplication, Chromosome 6p deletion, Chromosome 6p duplication, Chromosome 6q deletion, Chromosome 6q duplication, Chromosome 6q25 microdeletion syndrome, Chromosome 7p deletion, Chromosome 7p duplication, Chromosome 7q deletion, Chromosome 7q duplication, Chromosome 8p deletion, Chromosome 8p duplication, Chromosome 8p23.1 deletion, Chromosome 8q deletion, Chromosome 8q duplication, Chromosome 9 inversion—Not a rare disease, Chromosome 9p deletion, Chromosome 9p duplication, Chromosome 9q deletion, Chromosome 9q duplication, Chromosome Xq duplication, Chromosome Xq28 deletion syndrome, Diploid-triploid mosaicism, Distal chromosome 18q deletion syndrome, Emanuel syndrome, Jacobsen syndrome, Kleefstra syndrome, Koolen de Vries syndrome, Mosaic monosomy 18, Mosaic monosomy 22, Mosaic trisomy 13, Mosaic trisomy 14, Mosaic trisomy 22, Mosaic trisomy 7, Mosaic trisomy 8, Mosaic trisomy 9, Nablus mask-like facial syndrome, Pallister-Killian mosaic syndrome, Partial deletion of Y, Potocki-Shaffer syndrome, Proximal chromosome 18q deletion syndrome, Recombinant chromosome 8 syndrome, Ring chromosome 1, Ring chromosome 10, Ring chromosome 11, Ring chromosome 12, Ring chromosome 13, Ring chromosome 14, Ring chromosome 15, Ring chromosome 16, Ring chromosome 17, Ring chromosome 18, Ring chromosome 19, Ring chromosome 2, Ring chromosome 20, Ring chromosome 21, Ring chromosome 22, Ring chromosome 3, Ring chromosome 4, Ring chromosome 5, Ring chromosome 6, Ring chromosome 7, Ring chromosome 8, Ring chromosome 9, Smith-Magenis syndrome, Tetrasomy 9p, Tetrasomy X, Triploidy, Trisomy 13,Trisomy 17 mosaicism, Trisomy 2 mosaicism, Turner syndrome, Wolf-Hirschhorn syndrome, X-linked susceptibility to autism-4, Y chromosome infertility and Y chromosome pericentric inversion.

In some embodiments, chromosomal structural variants do not occur in every cell in the subject. In some embodiments, the cells with the chromosomal structural variant(s) are cancer cells in the subject. A subject with a cancer can have cancer cells with one or more chromosomal structural variants, while the non-cancerous cells of the subject do not have a chromosomal structural variant, or do not have the same chromosomal structural variants that are seen in the cancer cells of the subject.

Cancers are diseases caused by the proliferation of malignant neoplastic cells, such as tumors, neoplasms, carcinomas, sarcomas, blastomas, leukemias, lymphomas and the like. Cancers that can be analyzed using the methods described herein include solid tumors and liquid tumors. For example, cancers include, but are not limited to, mesothelioma, leukemias and lymphomas such as cutaneous T-cell lymphomas (CTCL), noncutaneous peripheral T-cell lymphomas, lymphomas associated with human T-cell lymphotrophic virus (HTLV) such as adult T-cell leukemia/lymphoma (ATLL), B-cell lymphoma, acute nonlymphocytic leukemias, chronic lymphocytic leukemia, chronic myelogenous leukemia, acute myelogenous leukemia, lymphomas, and multiple myeloma, non-Hodgkin lymphoma, acute lymphatic leukemia (ALL), chronic lymphatic leukemia (CLL), Hodgkin's lymphoma, Burkitt lymphoma, adult T-cell leukemia lymphoma, acute-myeloid leukemia (AML), chronic myeloid leukemia (CML), or hepatocellular carcinoma. Further examples include myelodisplastic syndrome, childhood solid tumors such as brain tumors, neuroblastoma, retinoblastoma, Wilms' tumor, bone tumors, and soft-tissue sarcomas, common solid tumors of adults such as head and neck cancers (e.g., oral, laryngeal, nasopharyngeal and esophageal), genitourinary cancers (e.g., prostate, bladder, renal, uterine, ovarian, testicular), lung cancer (e.g., small-cell and non-small cell), breast cancer, pancreatic cancer, melanoma and other skin cancers, stomach cancer, brain tumors, tumors related to Gorlin's syndrome (e.g., medulloblastoma, meningioma, etc.) and liver cancer.

Most cancers acquire one or more clonal chromosomal structural variants during the development of the cancer, which can be identified by the systems and methods of the disclosure. In many cases, recurrent chromosomal structural variants are associated with particular morphological and clinical disease characteristics. Structural variants in cancer cells can affect the expression and/or function of proto-oncogenes and tumor suppressors. Structural variants in cancer cells can also facilitate the progression of the cancer itself, as mutations and changes in gene expression caused by the chromosomal structural variant(s) promote increased growth and invasiveness of tumor cells, and tumor vascularization. Identifying the specific chromosomal structural variants in a cancer cells in a cancer sample allows for the more effective selection of cancer therapies. These therapies can be tailored to changes in gene expression and cancer pathologies associated with the particular chromosomal structural variants in the cancer cells. Thus, the rapid and effective identification of chromosomal structural variants in cancers is a critical piece of the cancer diagnostic and treatment arsenal.

In some embodiments, structural variants in cancer cells create novel fusion proteins which promote the progression of the cancer. A non-limiting, exemplary list of chromosomal structural variants that cause fusion proteins associated with cancers is described in Hasty, P. and Montagna, C. (2014) Mol. Cell. Oncol.: e29904 and shown below:

TABLE 2
Chromosomal structural variants creating fusion proteins associated with
cancers and targeted therapies

| Name | Breakpoint | Cancer | Therapy |
|---|---|---|---|
| BCR-ABL | t(9; 22) | Acute lymphoblastic | Imatinib |
| (Philadelphia | (q34; q11) | leukemia, acute | |
| chromosome) | | myelogenous leukemia, | |
| | | chronic myelogenous | |
| | | leukemia | |
| ALK-EML4 | Inv(2) | Non-small cell lung cancer | Crizotinib |
| c-ros oncogene | (p21; p23) | Non-small cell lung cancer, | Crizotinib |
| 1 (ROS1) and | | cholangiocarcinoma, | |
| additional | | glioblastoma multiforme, | |
| genes | | gastric adenocarcinoma and | |
| | | acute myelogenous leukemia | |
| AML1/ETO | t(8; 21) | acute myelogenous leukemia | General |
| | (q22; q22) | | chemotherapy |
| PML-RARA | t(15; 17) | acute myelogenous leukemia | ATRA and |
| | (q22; q21) | | arsenic oxide |
| Mixed lineage | | acute myelogenous leukemia | ATRA |
| leukemia | | | |
| (MLL) with | | | |
| various | | | |
| fusion partners | | | |
| PAX3-FOXO1 | t(2; 13) | alveolar rhabdomyosarcoma | Thapsigargin |
| | (q36; q14) | | |
| PAX7-FOXO1 | t(1; 13) | alveolar rhabdomyosarcoma | Therapeutics |
| | (p36; q14) | | targeting |
| | | | downstream |
| | | | pathways |
| FOXO3-MLL | t(6; 11) | alveolar rhabdomyosarcoma | ATRA |
| | (q21; q23) | and leukemia | |
| FOXO4-MLL | t(X; 11) | alveolar rhabdomyosarcoma | ATRA |
| | (q13; q23) | and leukemia | |
| FOXP1-PAX5 | t(3; 9) | Lymphoblastic leukemia | |
| | (p13; p13) | | |

Currently there are 21,477 documented gene fusions and 69,134 cases documented in the Cancer Genome Anatomy Project (cgap.nci.nih.gov/Chromosomes/Mitelman), all of which are envisaged as falling within the scope of the instant disclosure. Further non-limiting examples of chromosomal structural variants associated with cancers are described in Bernhein, A. Cytogenetics of cancers: from chromosome to sequence. 2010 Molecular Oncology 4(4): 309-322, and are shown in Table 3 below. Targeted therapies and clinical trials for therapies corresponding to known CSVs can be found at www.mycancergenome.org, the contents of which are incorporated by reference herein. In table 3, lists of variants and corresponding genes are listed in order.

TABLE 3
Examples of Chromosomal Variants Associated with Cancers

| Cancer | Variant (s) | Gene (s) | Targeted Therapy |
|---|---|---|---|
| Acute | t(1; 19)(q23; p13) | PBX1-TCF3 | Idelalisib |
| Lymphocytic | | | |
| Leukemia (ALL) | | | |
| L1/L2 Pre-B | | | |
| ALL L1/L2 B or | t(9; 22)(q34; q11) | ABL-BCR | Tyrosine kinase |
| biphenotypic | | | inhibitors (TM) |
| | | | including |
| | | | Imatinib, |
| | | | Dasatinib, |
| | | | Nilotinib, |
| | | | Bosutinib, |
| | | | Ponatinib |
| ALL L1/L2 | t(4; 11)(q21; q23) | AF4-MLL | — |
| biphenotypic | | | |
| ALL Ll/L2 | t(12; 21)(p13; q22) | TEL-AML1 | Autophagy |
| (child) | | | inhibitors, |
| | | | combination |
| | | | therapies |
| ALL L1/L2 | 50-60 chromosomes, hyper | _; IL3*IGH; | — |
| | diploidy; t(5; 14)(q31; q32); | CDKN2(p16); ABL- | |
| | del(9p),t(9p); t(9; 12)(q34; p13); | TEL; MLL-V; ETV6 | |
| | t(11; V)(q23; V); del(12p) | | |
| ALL L1/L3 | dup(6)(q22-q23); del(9)(p13); | MYB; PAX5 | — |
| ALL L1/L3 | episome(9q34.1)§ | NUP214-ABL1 | Imatinib |
| B (ALL3, | t(8; 14)(q24; q32) | IGH*MYC | Leuprolide and |
| Burkitt's | | | transplantation |
| leukemia/lymphoma) | | | |
| B (ALL3, | t(2; 8)(p12; q24); | IGK*MYCc | — |
| Burkitt's | t(8; 22)(q24; q11) | | |
| leukemia/lymphoma) | | | |
| Follicular | t(14; 18)(q32; q21) and variants | IGH*BCL2/IGK/IGL | Bc12 inhibitors |
| lymphoma to | | | (oblimersen, |
| large-cell diffuse | | | ABT-737, ABT- |
| lymphoma | | | 199) |
| Mantle-cell | t(11; 14)(q13; q32) | CCND1*IGH | Ibrutinib |
| lymphoma | | | |
| Marginal zone | t(1; 14)(p21; q32); 3 | BCL10*IGH; _ | — |
| lymphoma | | | |
| Marginal zone | t(11; 18)(q21; q21) | BIRC3-MALT1 | Rituximab, |
| lymphoma | | | chlorambucil |
| Large-cell diffuse | t(3; 14)(q27; q32), variants | BCL6*IGH, | — |
| lymphoma | | BCL6*V | |
| Large-cell diffuse | t(11; 14)(q13; q32) | CCND1*IGH | Ibrutinib |
| lymphoma | | | |
| Anaplastic large- | t(2; 5)(p23; q35), variants | ALK-NPM1 | ALK inhibitors |
| cell lymphoma | | | |
| Lymphocytic B | t(11; 14)(q13; q32) | CCND1*IGH | Ibrutinib |
| cell lymphoma, | | | |
| chronic | | | |
| lymphocytic | | | |
| leukemia | | | |
| Lymphocytic B | t(14; 19)(q32; q13); | IGH*BCL3; | — |
| cell lymphoma, | t(2; 14)(p13; q32); | BCL11A*IGH; | |
| chronic | del(11)(q23.1); del(13)(q14) | ATM; DLEU, miR- | |
| lymphocytic | | 16-1 & 15a | |
| leukemia | | | |
| Prolymphocytic T | inv(14)(q11q32); | TCRA/TCR D* | — |
| leukemia | t(14; 14)(q11; q32) | TCL1A | |
| Prolymphocytic T | t(7; 14)(q35; q32.1) | TCRB* TCL1A | — |
| leukemia | | | |
| Multiple | t(11; 14)(q13; q32) | CCND1*IGH | Ibrutinib |
| myeloma | | | |
| Multiple | t(4; 14)(p16; q32); del(6)(q21); | WHSC1-IGHG1; _; | — |
| myeloma | del(13)(q14) | DLEU, miR-16-1 & | |
| | | 15a | |
| Acute myeloid | t(8; 21)(q22; q22) | RUNX1-RUNX1T1 | — |
| leukemia (AML) | | | |
| M2 | | | |
| AML M3 and | t(15; 17)(q22; q11-12) | PML-RARA | Retinoid Acid |
| microgranular | | | |
| variant | | | |
| AML M3 | t(11; 17)(q23; q12) | PLZF-RARA | Retinoid Acid |
| (atypical) | | | |
| AML M4Eo | inv(16)(p13q22) ou; | CBFB-MYH11 | — |
| | t(16; 16)(p13; q22 | | |
| AML M5a and | t(9; 11)(p22; q23); t(11q23; V) | MLL-MLLT3; MLL | — |
| other AML | | multiple partners | |
| | | including MLL | |
| Acute | t(1; 22)(p13; q13) | RBM15-MKL1 | — |
| megakaryoblastic | | | |
| leukemia | | | |
| AML, | t(3; 3)(q21; q26) or variants | RPN1-EVI1 | — |
| myelodysplastic | | | |
| syndromes | | | |
| (MDS) | | | |
| AML, MDS | t(3; 5)(q25; q34); | MLF1-NPM1; | — |
| | t(5; 12)(q33; p13); −5/del(5q); | PDGFRB-ETV6; | |
| | t(6; 9)(p23; q34); | RPS14; DEK- | |
| | t(7; 11)(p15; p15); −7 ou del(7q); | NUP214; HOXA9- | |
| | 8; t(8; 16)(p11; p13); t(9; 12)(q34; | NUP98; numerous | |
| | p13); t(12; 13)(p13; q12.3); | genes; _; MOZ-CBP; | |
| | t(12; 22)(p13; q13); | ETV6-ABL; ETV6- | |
| | t(12; V)(p13; V), del(12p); | CDX2; ETV6-NM1; | |
| | (16; 21)(p11; q22); del(20q) | ETV6L-V; FUS- | |
| | | ERG; _ | |
| Alkylating agent- | −5 ou del(5q); −7 ou del(7q) | — | — |
| and irradiation- | | | |
| induced leukemia | | | |
| Anti | t(11q23; V) | MLL-V | — |
| topoisomerase II | | | |
| induced leukemia | | | |
| Chronic myeloid | t(9; 22)(q34; q11) | BCR-ABL1 | Imatimib, 2nd |
| leukemia (CML) | | | generation |
| | | | tyrosine kinase |
| | | | inhibitor (TM) |
| Lymphoblastic | t(9; 22), +8,+Ph, +19, i(17q) | BCR-ABL1 | Imatimib, 2nd |
| acutisation of | | | generation TKI |
| CML | | | |
| Polycytemia vera | +9p; del(20q) | — | — |
| MDS/MPD | t(8; 9(p21; p24) | PCM1-JAK2 | JAK inhibitors |
| Chronic | t(5; 12)(q33; p13) | PDGFRB-TEL | Imatinib |
| myelomonocytic | | | |
| leukemia | | | |
| 5q- syndrome | del(5q) | RPS14 | — |
| Breast cancer | amp(1)(q32.1); amp(20)(q12) | IKBKE; NCOA3 | — |
| Breast and | amp(6)(q25.1) | ESR1 | Tamoxifen |
| various cancers | | | |
| Breast cancer | amp(17)(q21.1) | ERBB2 (HER2) | Trastuzumab, |
| | | | Lapatinib |
| Breast and | t(12; 15)(p13; q25) | ETV6-NTRK3 | Trk inhibitors |
| various cancers | | | |
| Colon cancer | del(4)(q12); del(5)(q21-q22) | REST; APC | — |
| Hepatocellular | amp(11)(q13-q22); | BIRC2; YAP1 | — |
| carcinoma | amp(11)(q13-q22) | | |
| Lung cancer | amp(1)(p34.2) | MYCL1 | — |
| Lung cancer | inv(2)(p22-p21p23) | EML4-ALK | ALK inhibitors, |
| (non-small-cell) | | | Alectinib, |
| | | | Crizotinib |
| Lung, head and | amp(3)(q26.3) | DCUN1D1 | — |
| neck cancers | | | |
| Lung cancer | amp(7)(p12) | EGFR | Cetircimab, |
| (non-small-cell) | | | Panitumumab, |
| | | | Gefitinib, |
| | | | Erlotinib |
| Lung cancer | amp(14)(q13) | NKX2-1 | — |
| (non-small-cell) | | | |
| Ovarian cancer | amp(1)(q22); mp(3)(q26.3) | RAB25; PIK3CA | — |
| Ovarian, breast | amp(11)(q13.5); | EMSY1; RPS6KB1 | — |
| cancers | amp(17)(q23.1) | | |
| Prostate cancer | amp(X)(q12) | AR | — |
| Prostate cancer | del(21)(q22.3q22.3) | TMPRSS2*ERG | |
| Renal carcinoma | .+7q31; .+17q; t(X; 1)(p11; p34); | MET; _; PSF-TFE3; | — |
| papillary | t(X; 1)(p11.2; q21.2) | PRCC-TFE3 | |
| Thyroid cancer | t(2; 3)(q12-q14; p25); | PAX8-PPARG; | — |
| follicular | inv(10)(q11.2q11.2); | RET-NCOA4; RET- | |
| | inv(10)(q11.2q21) | CCDC6 | |
| Ewing's sarcoma | t(11; 22)(q24.1-q24.3; q12.2); | FLI1-EWSR1; | — |
| | t(21; 22)(q22.3 ; q12.2) | ERG-EWSR1 | |
| Rhabdomyosarcoma | t(1; 13)(p36; q14); | PAX7-FKHR; | — |
| (alveolar) | t(1; 13)(p36; q14); | PAX7-FKHR; | |
| | t(2; 13)(q37; q14) | PAX3-FKHR | |
| Chondrosarcoma | t(9; 17)(q22; q11) | RBP56-CHN | — |
| (extrasqueletical) | | | |
| Chondrosarcomas | t(9; 22)(q22; q12) | EWS-CHN | — |
| (myxoid) | | | |
| Desmoplastic | t(11; 22)(p13; q12) | WT1-EWS | — |
| tumors | | | |
| Clear cell | t(12; 22)(q13; q12) | ATF1-EWS | — |
| sarcomas | | | |
| Liposarcomas | t(12; 16)(q13; p11) | CHOP-FUS | — |
| Liposarcomas | t(12; 16)(q13; p11) | CHOP-FUS | — |
| (myxoid) | | | |
| Dermatofibrosarcomas | t(17; 22)(q22; q13) | COL1A1-PDGFB | — |
| protuberans | | | |
| Alveolar soft part | der(17)4X; 17)(p11; q25) | ASPSCR1-TFE3 | — |
| sarcomas | | | |
| Synovialosarcomas | t(X; 18)(p11.2; q11.2) | SYT-SSX1/SSX2-SYT | — |
| Malignant | amp(3)(p14.2-p14.1) | MITF | — |
| melanoma | | | |
| Glioma | amp(1)(q32) | MDM4 | — |
| Astrocytoma, | .+7 | — | — |
| glioblastoma | | | |
| Anaplastic | del(19q); del(1p) | — | — |
| oligodendroglioma | | | |
| Medulloblastoma | amp(2)(p24.1); del(6)(q23.1); | MYCN; WNT; | — |
| | amp(8)(q24.2); del(9)(p21); | MYC; | |
| | i(17q) | CDKN2A/CDKN2B; | |
| | | P53 | |
| Neuroblastoma | amp(2)(p24.1); del(1p) | MYCN; _ | — |
| Neuroblastoma | amp(2)(p23.1) | ALK | ALK inhibitors |
| | | | (crizotinib, |
| | | | ceritinib, |
| | | | alectinib, |
| | | | brigatinib, |
| | | | lorlatinib) |
| Renal-cell cancer | del(3p26-p25) | VHL | — |
| Retinoblastoma | del(13)(q14.2); amp(1)(q32); | RB1; MDM4; RB | — |
| | del(13)(q14) | | |
| Testicular germ- | +12p | — | — |
| cell tumor | | | |
| Wilms' tumor | del(11p); del(X)(q11.1) | WT1; FAM123B | — |
| Various cancers | +1q; del(3p); del(6q); deh(11q); | — | — |
| | +17q | | |
| Various cancers | amp(5)(p13); amp(6)(p22); | SKP2; E2F3; MET; | — |
| | amp(7)(q31); amp(8)(p11.2); | FGFR1; MYC; | |
| | amp(8)(q24.2); del(9)(p21); | CDKN2A/CDKN2B; | |
| | amp(11)(q13); del(11)(q22- | CCND1; ATM; | |
| | q23); amp(12)(p12.1); | KRAS; MDM2; | |
| | amp(12)(q14.3); amp(12)(q15); | DYRK2; GPC5; | |
| | amp(13)(q32); del(17)(q11.2); | NF1; CCNE1; | |
| | amp(19)(q12); amp(20)(q13) | AURKA | |
| Various cancers | amp(7)(p12) | EGFR | Cetuximab, |
| | | | Panitumumab, |
| | | | Gefitinib, |
| | | | Erlotinib, |
| | | | Lapatinib |
| Various cancers | deh(10)(q23.3) | PTEN | PARP inhbitors |
| Various cancers | amp(12)(q14) | CDK4 | Palbociclib, |
| | | | Ribociclib |
| Various cancers | amp(17)(q21.1) | ERBB2 (HER2) | Trastuzumab, |
| | | | Lapatinib, |
| | | | Pertuzamab, |
| | | | Afatinib |
| Various cancers | del(17)(p13.1) | TP53 | rituxumab, |
| | | | lenalidomide, |
| | | | idelalisib |
| Various cancers | Del(5)(q31q33) | | lenalidomide |

In some embodiments, chromosomal structural variants in cancer cells lead to changes in gene regulation and gene expression, which contribute to the progression of the cancer. A chromosomal structural variant can lead to the downregulation of one or more the tumor suppressors, which are genes that protect the cell from cancer. For example, a chromosomal structural variant with a break point near a tumor suppressor can separate the coding sequence of the tumor suppressor from a regulatory element. Alternatively, or in addition, a chromosomal structural variant can lead to the conversion of one or more proto-oncogenes into an oncogene which promotes cancer progression. For example, a chromosomal structural variant with a break point near a proto-oncogene can bring the proto-oncogene into proximity of a novel regulatory element, leading to upregulated expression. Exemplary tumor suppressors that can be down regulated by the chromosomal structural variants of the disclosure include, but are not limited to, p53, Rb, PTEN, INK4, APC, MADR2, BRCA1, BRCA2, WT1, DPC4 and p21. Exemplary oncogenes that can be upregulated by the chromosomal structural variants of the disclosure include, but are not limited to, Abl1, HER-2, c-KIT, EGFR, VEGF, B-Raf, Cyclin D1, K-ras, beta-catenin, Cyclin E, Ras, Myc and MITF. All chromosomal structural elements which affect proto-oncogenes and tumor suppressor genes are envisaged as within the scope of the systems and methods of the disclosure.

### Chromosomal Conformational Capture

Provided herein are systems and methods that use chromosomal conformation capture techniques to identify one or more chromosomal structural variants in a subject.

The terms “chromosomal conformational capture” and “chromosome conformation analysis” are used interchangeably herein.

The methods of the disclosure can use standard chromatin conformation data, such as Hi-C data, generated from a tissue sample (e.g. cancerous or normal tissues or cells). The computational methods involves the training of one or more machine learning models, which can be used in more than one of the major applications. The one or more machine learning models chosen may include deep learning models, gradient descent models, graph network models, neural network models, support vector machine models, expert system models, decision tree models, logistic regression models, clustering models, Markov models, Monte Carlo models, or other machine learning models, as well as models which fit observed data to probabilistic models such as likelihood models. The one or more machine learning models can include a supervised machine learning model trained based on labeled training data, and/or can include an unsupervised machine learning model trained based on unlabeled training data. Training data, such as for example, the labeled training data and/or the unlabeled training data, can be generated from real biological samples, simulated genomes which may have simulated mutations, or can be generated using another algorithm, such as algorithms used in a generative adversarial network. The training data comprises chromatin conformation data or data derived from it (such as a contact matrix, and may be normalized, filtered, compressed, or smoothed) and clinical or biological information about the effects, properties, implications, or outcomes associated with the data.

In some embodiments of the systems and methods of the disclosure, the systems and methods comprise one or more machine learning models that are trained using chromosomal conformation capture data. In some embodiments, the one or more machine learning models are trained using experimentally determined chromosomal conformational capture data. In some embodiments, the one or more machine learning models are trained using simulated chromosomal conformational capture data. In some embodiments, the one or more machine learning models are trained using a combination of experimentally determined and simulated chromosomal conformational capture data.

In some embodiments, the chromosomal conformational capture data used to train the one or more machine learning machine learning models comprises experimentally determined chromosomal conformational capture data. In some embodiments, the experimentally determined chromosomal conformational capture data comprises a plurality of sets of reads from healthy subjects. In some embodiments, the experimentally determined chromosomal conformational capture data comprises a plurality of sets of reads from subjects with known chromosomal structural variants.

Chromosomal conformational data is generated by chemically cross-linking regions of the genome that are in close spatial proximity. The cross linked DNA is then restriction enzyme digested and ligated to generate chromatin/DNA complexes which can be identified by high-throughput sequencing. The resultant sequence reads are mapped to a genome, for example a reference genome, to determine the frequency with which each interaction occurs within the population of cells that was used to generate the initial sample. When two loci are in close spatial proximity, they will generate more reads that comprise DNA sequences that map both loci than if the two loci are not in close spatial proximity.

Experimentally determined chromosomal conformational capture data may form part of an input file used by a system to carry out the methods described herein. The set of reads may be generated by any suitable method based on chromatin interaction techniques or chromosome conformation analysis techniques. Chromosome conformation analysis techniques that may be used in accordance with the embodiments described herein may include, but are not limited to, Chromatin Conformation Capture (3C), Circularized Chromatin Conformation Capture (4C), Carbon Copy Chromosome Conformation Capture (5C), Chromatin Immunoprecipitation (ChIP; e.g., cross-linked ChIP (XChIP), native ChIP (NChIP)), ChIP-Loop, genome conformation capture (GCC) (e.g., Hi-C, 6C), Capture-C, Split-pool barcoding (SPLiT-seq), Nuclear Ligation Assay (NLA), Single-cell Hi-C (scHi-C), Combinatorial Single-cell Hi-C, Concatamer Ligation Assay (COLA), Cleavage Under Targets and Release Using Nuclease (CUT& RUN), in vitro proximity ligation (e.g. Chicago®), in situ proximity ligation (in situ Hi-C), proximity ligation followed by sequencing on an Oxford Nanopore machine (Pore-C), proximity ligation sequenced on a Pacific Biosciences machine (SMRT-C), DNase Hi-C, Micro-C and Hybrid Capture Hi-C. In some embodiments, the dataset is generated using a genome-wide chromatin interaction method, such as Hi-C.

In some embodiments, chromosomal conformational data can be generated from a population of cells. In some embodiments, chromosomal conformational capture data is generated by Chromatin Conformation Capture (3C). 3C is used to analyze the organization of chromatin in a cell by quantifying the interactions between genomic loci that are nearby in 3-D space. 3C quantifies interactions between a single pair of genomic loci. In some embodiments, chromosomal conformational capture data is generated by Circularized Chromatin Conformation Capture (4C). 4C captures interactions between one locus and all other genomic loci. In some embodiments, chromosomal conformational capture data is generated by Carbon Copy Chromosome Conformation Capture (5C). 5C detects interactions between all restriction fragments within a given region. In some embodiments, the region is one megabase or less. In some embodiments, chromosomal conformational capture data is generated by Chromatin Immunoprecipitation (ChIP; e.g., cross-linked ChIP (XChIP), native ChIP (NChIP)). In some embodiments, chromosomal conformational capture data is generated by ChIP-Loop. In some embodiments, chromatin immumoprecipitation based methods incorporate chromatin immunoprecipitation (chIP) based enrichment and chromatin proximity ligation to determine long range chromatin interactions. In some embodiments, chromosomal conformational capture data is generated by Hi-C. Hi-C uses high-throughput sequencing to find the nucleotide sequence of fragments that map to both partners in all interacting pairs of loci. In some embodiments, chromosomal conformational capture data is generated by Capture-C. Capture-C selects and enriches for genome-wide, long-range contacts involving active and inactive promoters. In some embodiments, chromosomal conformational capture data is generated by SPLIT-seq. SPLIT-seq is a technique that can be used to transcriptome profile single cells. In some embodiments, chromosomal conformational capture data is generated by Nuclear Ligation Assay (NLA). Similar to 3C, NLA can be used to determine the circularization frequencies of DNA following proximity based ligation. In some embodiments, chromosomal conformational capture data is generated by Concatamer Ligation Assay (COLA). COLA is a Hi-C based protocol that uses the CviJI restriction enzyme to digest chromatin. In some embodiments, using COLA results in smaller fragments compared to traditional Hi-C. In some embodiments, chromosomal conformational capture data is generated by Cleavage Under Targets and Release Using Nuclease (CUT& RUN). CUT & RUN uses a targeted nuclease strategy for high-resolution mapping of DNA binding sites. For example, CUT&RUN can use an antibody-targeted chromatin profiling method in which a nuclease tethered to protein A binds to an antibody of choice and cuts immediately adjacent DNA, releasing DNA bound to the antibody target. CUT & RUN can be carried out in situ. CUT & RUN can produce precise transcription factor or histone modification profiles, as wells as mapping long-range genomic interactions. In some embodiments, chromosomal conformational capture data is generated by DNase Hi-C. DNase Hi-C uses DNase I for chromatin fragmentation, and can overcome restriction enzyme related limitations in conventional Hi-C protocols. In some embodiments, chromosomal conformational capture data is generated by Micro-C. Micro-C using micrococcal nuclease to fragment chromatin into mononucleosomes. In some embodiments, chromosomal conformational capture data is generated by Hybrid Capture Hi-C. Hybrid Capture Hi-C combines targeted genomic capture and with Hi-C to target selected genomic regions.

In some alternative embodiments, chromosomal conformational capture data can be generated from a single cell. For example, the chromosomal conformation capture data can be generated using Single-cell Hi-C (scHi-C) or Combinatorial Single-cell Hi-C. Single-cell Hi-C is an adaptation of Hi-C to single-cell analysis by including in-nucleus ligation. Combinatorial single-cell Hi-C is a modified single-cell Hi-C protocol that adds unique cellular indexing to measure chromatin accessibility in thousands of single cells per assay.

In some embodiments, chromosomal conformational capture data can be generated from a proximity ligation based protocol that is carried out in situ, i.e. in intact nuclei.

In some embodiments, chromosomal conformational capture data can be generated from a proximity ligation based protocol that is carried out in vitro. Exemplary in vitro based protocols include Chicago® from Dovetail Genomics, which using high molecular weight DNA as a starting material. In some embodiments, the input DNA is about 20-200 kbp. In some embodiments, the input DNA is about 50 kbp.

In some embodiments, generating the chromosomal conformation capture data comprises: (a) contacting a sample from a subject with a stabilizing agent, wherein said sample comprises nucleic acids; (b) cleaving the nucleic acids into a plurality of fragments comprising at least a first segment and a second segment; (c) attaching the first segment and the second segment at a junction to generate a plurality of fragments comprising attached segments; (d) obtaining at least some sequence on each side of the junction of the plurality of fragments comprising attached segments to generate a plurality of reads; and (e) applying any of the machine learning models described herein to the plurality of reads from the subject.

In some embodiments, the nucleic acids comprise genomic DNA. For example, the nucleic acids comprise genomic DNA extracted from a sample from the subject.

In some embodiments, the stabilizing agent comprises ultraviolet light or a chemical fixative. Exemplary chemical fixatives include formaldehyde.

In some embodiments, cleaving the nucleic acids comprises mechanical cleavage or enzymatic cleavage. Mechanical cleavage can be accomplished by shearing, such as with a sonicator. Exemplary methods of enzymatic cleavage include digestion by restriction enzyme.

In some embodiments, attaching the first segment and the second segment comprises ligation. For example, the methods can include intramolecular ligation to attach fragments, before reversing the stabilizing or cross linking agent.

Chromosomal conformational capture data used by the methods and systems of the disclosure can be generated using any sequencing methods or next generation sequencing platform known in the art. For example, chromosomal conformational capture data may be generated by proximity ligation followed by sequencing on an Oxford Nanopore machine (Pore-C), a Pacific Biosciences machine (SMRT-C), a Roche/454 sequencing platform, ABI/SOLID platform, or an Illumina/Solexa sequencing platform.

In some embodiments of the systems and methods of the disclosure, the methods comprise mapping reads generated by chromosomal conformational capture onto a genome. In some embodiments, the sets of reads may be aligned with the genome any suitable alignment method, algorithm or software package known in the art. Suitable short read sequence alignment software that may be used to align the set of reads with an assembly include, but are not limited to, BarraCUDA, BBMap, BFAST, BLASTN, BLAT, Bowtie, HIVE-hexagon, BWA, BWA-PSSM, BWA-mem, CASHX, Cloudburst, CUDA-EC, CUSHAW, CUSHAW2, CUSHAW2-GPU, CUSHAW3, drFAST, ELAND, ERNE, GASSST, GEM, Genalice MAP, Geneious Assembler, GensearchNGS, GMAP and GSNAP, GNUMAP, IDBA-UD, ISAAC, LAST, MAQ, mrFAST and mrsFAST, MOM, MOSAIK, Novoalign & NovoalignCS, NextGENe, NextGenMap, Omixon, PALMapper, Partek, PASS, PerM, PRIMEX, QPalma, RazerS, REAL, cREAL, RMAP, INA, RTG Investigator, Segemehl, SeqMap, Shrec, SHRIMP, SLIDER, SOAP, SOAP2, SOAP3, SOAP3-dp, SOCS, SSAHA, SSAHA2, Stampy, STORM, subread and Subjunc, Taipan, UGENE, VelociMapper, XpressAlign, and Zoom.

In some embodiments of the systems and methods of the disclosure, the methods further comprise filtering out reads that align poorly to a reference genome prior to applying the machine learning models described herein. In some embodiments, the method comprises filtering out reads that align poorly in the training dataset. In some embodiments, the method comprises filtering out reads that align poorly in the data from the subject. In some embodiments, filtering out reads comprises mapping the chromosomal conformational capture reads onto a reference genome and filtering out the low quality alignment data. For example, reads can be aligned to a reference genome using BWA-mem, and low quality alignment data with less than MQ 20 is excluded.

In some embodiments, the one or more machine learning models are trained using simulated chromosomal conformational capture data. In some embodiments, the simulated chromosomal conformational capture data simulates one or more chromosomal structural variants. In some embodiments the simulated chromosomal conformational capture data simulates chromosomal conformational capture data from subjects who do not have chromosomal structural variants. In some embodiments, the simulated chromosomal conformational capture data from subjects who do not have chromosomal structural variants comprises all regions of the genome of the subject.

Methods of simulating chromosomal conformation capture data are described herein. Given the high costs of sequence large numbers of samples, it is cost effective and advantageous to train machine learning models used in the methods disclosed herein using simulated chromosomal conformation capture data that covers the full genome of the subject. Further, using simulated data to model full genomes of subjects without chromosomal structural variants t prevents over-fitting of data during training of the machine learning models, and ensures that the machine learning models disclosed herein will recognize the “null” model, i.e. when no chromosomal structural variant is present for all regions in the genome of the subject.

In some embodiments of the methods and systems of the disclosure, chromosomal conformational capture data is represented as a geometric data structure. Chromosomal conformational capture data represented as a geometric data structure can be used to train the machine learning models described herein. Chromosomal conformational capture data from a subject, for example a subject who has, or is suspected of having, a chromosomal structural variant, can be represented as a geometric data structure and the chromosomal structural variant identified using the machine learning models described herein.

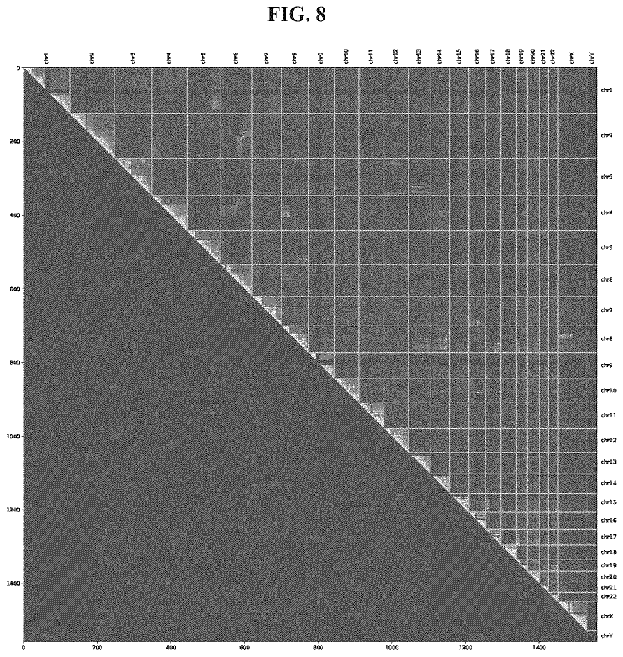

In some embodiments of the methods and systems of the disclosure, chromosomal conformational capture data is represented as a matrix. In some embodiments, the matrix is a contact matrix. A contact matrix is a matrix that stores interaction data between pairs of loci in a genome (e.g. a reference genome species-matched to the subject). A contact matrix of the disclosure can be generated by the following steps: (i) performing a chromosome conformation analysis technique on a sample from the subject to generate a set of reads; (ii) aligning the set of reads from the subject to the reference genome; and (iii) transforming the aligned set of reads into a contact matrix. In some embodiments, transforming the aligned set of reads into a contact matrix further comprises (iv) binning the reads into regions of the genome; and (v) normalizing the matrix by the size of the bins, the overall abundance of contact interactions in bins, and/or the frequency of the appearance of restriction motifs or other DNA sequences of interest present in those bins. Alternatively, or in addition, the matrix can be corrected for experimental, biological, technical, or other forms of noise or error using iterative correction, weighting, noise modeling, translation of signal to the percent domain, use of statistical measures such as mean, median, or percentiles, the application of low-pass, high-pass, or mid-pass filters, or other statistical techniques. In an exemplary contact matrix of the disclosure, each row and column corresponds to a position in a genome (e.g. a reference genome corresponding to the genome of the subject), binned to a specific nucleotide resolution, and the value entered into each cell of the matrix corresponds to the number of chromosomal conformational capture reads that map to both the row and column genome positions (i.e., the interaction frequency of those two loci). In some embodiments, the contact matrix is normalized for the number of restriction motifs present in the bins, and iterative correction is performed. An exemplary visualization of a contact matrix is shown in FIG. 8.

In some embodiments, the genome of the subject is divided into bins of contiguous nucleotides, and each cell in the contact matrix represents a bin of contiguous nucleotides. In some embodiments, each cell of the contact matrix comprises between 100 bp and 20,000,000 bp of the genome of the subject. In some embodiments, each cell of the contact matrix comprises between 10,000 bp and 10,000,000 bp of the genome of the subject. In some embodiments, each cell of the contact matrix comprises 5,000,000 bp of the genome of the subject, 4,000,000 bp of the genome of the subject, 3,000,000 bp of the genome of the subject, 2,000,000 bp of the genome of the subject, 1,000,000 bp of the genome of the subject, 500,000 bp of the genome of the subject, 400,000 bp of the genome of the subject, 300,000 bp of the genome of the subject, 200,000 bp of the genome of the subject, 100,000 bp of the genome of the subject, 10,000 bp of the genome of the subject, 5,000 bp of the genome of the subject, 1,000 bp of the genome of the subject, 500 bp of the genome of the subject or 100 bp of the genome of the subject.

In some embodiments, each cell of the contact matrix comprises 3,000,000 bp of the genome of the subject.

In some embodiments, each cell of the contact matrix comprises 1,000 bp of the genome of the subject.

In some embodiments, each cell of the contact matrix comprises 100 bp of the genome of the subject.

In some embodiments, the contact matrix comprises the entire genome of the subject.

In some alternative embodiments, the contact matrix comprises a portion of the genome of the subject (e.g. a chromosome, or a portion of a chromosome). In some embodiments, the contact matrix comprises a portion of the genome of the subject that corresponds to a bounding box around a chromosomal structural variant that has been identified using the systems and methods of the disclosure.

In some embodiments, the contact matrix is an averaged contact matrix, a median contact matrix, or a contact matrix with a percentile cut-off. In some embodiments, the averaged contact matrix has a resolution of between 100 bp per cell and 10,000,000 bp per cell.

In some embodiments of the methods and systems of the disclosure, chromosomal conformational capture data is represented as an image. In some embodiments, the contact matrix is represented as an image. Exemplary image representations comprise heat maps. In an exemplary heat map, genomic location, binned to a particular resolution, is plotted along both X and Y coordinates, and the opacity of each cell or pixel is directly related to the frequency of interactions represented by the loci at the X and Y coordinate positions.

In some embodiments of the methods and systems of the disclosure, chromosomal conformational capture data is represented as a geometric data structure. In some embodiments, the geometric data structure comprises a k-dimensional tree (a k-d tree). K-d trees are space-partitioning data structures that will be familiar to a person of ordinary skill in the art.

In some embodiments, the k-d tree is a two dimensional k-d tree. For example, data from a contact matrix can be transformed into a k-d tree.

In some embodiments, a first axis of the 2-d k-d tree represents a first genomic region, and a second axis of the k-d represents a second genomic location, and the k-d tree represents a frequency of links between any two genomic locations in each of the sets of reads from either a set of reads used to train a machine learning models (e.g., a classifier machine learning model) of the disclosure, a set of reads from a subject, or both.

In a 2D k-d tree of the disclosure, both axes represent genomic locations, for example in a reference genome corresponding to the subject, and the information contained in the k-d comprises the number of read pairs that map between each region on each axis (the linkage frequencies). This arrangement allows for the discernment of all structural relationships among all loci in a genome, even regions for which there is not any actually data, in a computationally efficient manner using O(log(n)).

One advantage of a k-d tree is that, unlike a traditional contact matrix, it can be accessed at an arbitrary resolution without any need to recompute the contact matrix at a new resolution. For example, using the methods of the disclosure, the entire k-d tree can first be interrogated at a genome-wide scale to identify regions of interest that may comprise chromosomal structural variants. Then, the regions of interest can be interrogated at increasingly fine resolution until the borders of the chromosomal structural variants are defined to an appropriate resolution. In some embodiments, the resolution comprises a 500,000 bp resolution, a 100,000 bp resolution, a 50,000 bp resolution, a 10,000 bp resolution, a 1,000 bp resolution, a 500 bp resolution or a 100 bp resolution. The resolution at which to interrogate the k-d can be tailored to known chromosomal structural variants. For example, large variants can be identified with coarser resolution, while smaller variants require finer resolution. Using these techniques, the borders of chromosomal structural variants can be resolved to within 500,000 bp, within 100,000 bp, within 50,000 bp, within 10,000 bp, within 1,000 bp, within 500 bp or within 100 bp. This can indicate, for example, whether or not a chromosomal structural variant is likely to affect the function of a gene at its border, for example by truncating the gene. Thus, k-d trees provide superior resolution and scaling, and requires less intensive computations than traditional contact matrices.

### Machine Learning Models

Disclosed herein are methods of treating a subject with a chromosomal structural variant. In some embodiments, the methods comprise: (a) receiving a test set of reads from a sample from the subject; (b) aligning the test set of reads from the subject to a reference genome; (c) training a machine learning model to distinguish between sets of reads from healthy subjects and sets of reads corresponding to known chromosomal structural variants; (d) applying the machine learning model to the mapped set of reads from the subject after training the machine learning model; (e) computing a likelihood that the subject has a known chromosomal structural variant based on applying the machine learning model to the mapped set of reads from the subject; and (f) generating a karyotype of the subject based on the likelihood the subject has the known chromosomal structural variant; wherein the test set of reads, the sets of reads from healthy subjects and the sets of reads corresponding to known chromosomal structural variants are generated by a chromosome conformation analysis technique.

In some embodiments, the methods comprise generating geometric data structures from the test set of reads, the sets of reads from healthy subjects and sets of reads corresponding to known chromosomal structural variants. Machine learning models can be trained to identify, or discriminate between, geometric data structures corresponding to sets of reads from healthy subjects and sets of reads corresponding to known chromosomal structural variants. Trained machine learning models as described herein can be applied to geometric data structures from the test set of reads for the subject to identify chromosomal structural variants in the subject.

Provided herein are systems for applying out the methods of the disclosure for identifying structural variants in a subject.

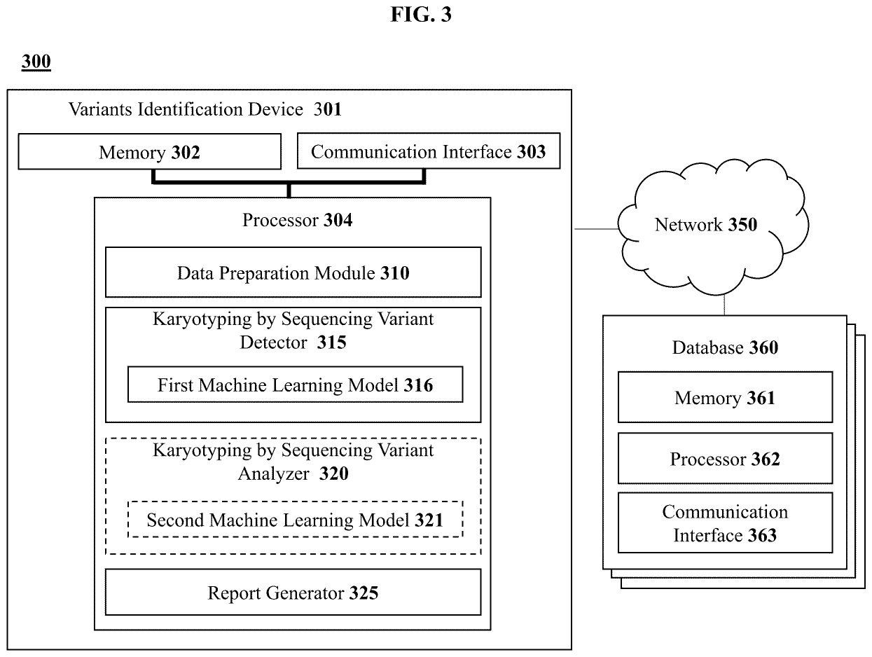

FIG. 3 is a block diagram that illustrates a variants identification system 300, according to an embodiment. The variants identification system 300 can include a variants identification device 301 (also referred to herein as “the variants detection device”) used to generate and report detected variants with significance in response to information from a sample or set of samples (e.g., a set of clinical samples, a set of research samples, and/or the like). Information from a sample or set of samples includes sequencing information produced by chromosomal capture techniques, and/or contact matrices and the like. The information from the sample or the set of samples can be in form of computer data stored in a memory described hereby. The variants identification device 301 can be a hardware-based computing device and/or a multimedia device, such as, for example, a computer, a laptop, a smartphone, a tablet, and/or the like. The variants identification device 301 can be communicatively coupled to a network 350 and further communicate, via the network 350, with a set of databases 360.

The variants identification device 301 includes a memory 302, a communication interface 303, and a processor 304. The variants identification device 301 can receive a set of sample information from a data source. The data source can include, for example, the set of databases 360, a file system, a peripheral device communicatively coupled to the variants identification device 301, and/or the like. The variants identification device 301 can receive the set of sample information from the data source in response to a user of the variants identification device 301 providing an indication to begin identification of variants of the set of samples.

The memory 302 of the variants identification device 301 can be, for example, a memory buffer, a random access memory (RAM), a read-only memory (ROM), a hard drive, a flash drive, a secure digital (SD) memory card, an external hard drive, a universal flash storage (UFS) device, and/or the like. The memory 302 can store, for example, one or more software modules and/or code that includes instructions to cause the processor 304 to perform one or more processes or functions (e.g., a first machine learning model 316, a second machine learning model 321, a report generator 325, and/or the like). The memory 302 can store a set of files associated with (e.g., generated by executing) the first machine learning model 316 and/or the second machine learning model 321. The set of files associated with the first machine learning model 316 and/or the second machine learning model 321 can include data generated by the first machine learning model 316 and/or the second machine learning model 321 during the operation of the variants identification device 301. For example, the set of files associated with the first machine learning model 316 and/or the second machine learning model 321 can include temporary variables, return memory addresses, variables, a graph of a machine learning model (e.g., a set of arithmetic operations or a representation of the set of arithmetic operations used by the machine learning model), the graph's metadata, assets (e.g., external files), electronic signatures (e.g., specifying a type of the machine learning model being exported, and the input/output tensors), and/or the like, generated during the operation of the machine learning model.

The communication interface 303 of the variants identification device 301 can be a hardware component of the variants identification device 301 operatively coupled to the processor 304 and/or the memory 302. The communication interface 303 can be operatively coupled to and used by the processor 304. The communication interface 303 can be, for example, a network interface card (NIC), a Wi-Fi™ module, a Bluetooth® module, an optical communication module, and/or any other suitable wired and/or wireless communication interface. The communication interface 303 can be configured to connect the variants identification device 301 to the network 350. In some instances, the communication interface 303 can facilitate receiving or transmitting data via the network 350. More specifically, in some implementations, the communication interface 303 can facilitate receiving/transmitting the information from the sample or set of samples from/to the set of databases, each communicatively coupled to the variants identification device 301 via the network 350. In some instances, data received via communication interface 303 can be processed by the processor 304 or stored in the memory 302, as described in further detail herein.

The processor 304 can be, for example, a hardware based integrated circuit (IC) or any other suitable processing device configured to run or execute a set of instructions or a set of codes. For example, the processor 304 can include a general purpose processor, a central processing unit (CPU), an accelerated processing unit (APU), a field programmable gate array (FPGA), a graphics processing unit (GPU), a neural network processor (NNP), and/or the like. The processor 304 is operatively coupled to the memory 302 through a system bus.

The network 350 can be a digital telecommunication network of servers and/or compute devices. The servers and/or computes device on the network can be connected via one or more wired or wireless communication networks (not shown) to share resources such as, for example, data or computing power. The wired or wireless communication networks between servers and/or compute devices of the network 350 can include one or more communication channels, for example, a radio frequency (RF) communication channel(s), a fiber optic commination channel(s), an electronic communication channel(s), and/or the like. The network 350 can be, for example, the Internet, an intranet, a local area network (LAN), a wide area network (WAN), a metropolitan area network (MAN), an/or the like.

The set of databases 360 can include databases, such as external hard drives, external compute device, cloud database services, and/or the like. The set of databases 360 each having a memory 361, a communication interface 363, and a processor 362, that can be structurally and/or functionally similar to the memory 302, the communication interface 303, and the processor 304, respectively. The set of databases 360 can be communicatively coupled to the variants identification device via the network 350.

The processor 304 can include a data preparation module 310, a karyotyping by sequencing variant detector 315, a first machine learning model 316, and a report generator 325. The processor 304 can optionally include a karyotyping by sequencing variant analyzer 320, a second machine learning model 321. Each of the data preparation module 310, the karyotyping by sequencing variant detector 315, the first machine learning model 316, the karyotyping by sequencing variant analyzer 320, the second machine learning model 321, and the report generator 325 can be software stored in the memory 302 and executed by the processor 304. For example, a code to cause the first machine learning model 321 to generate a layout from a document can be stored in memory 302 and executed by the processor 304. Similarly, each of the data preparation module 310, the karyotyping by sequencing variant detector 315, the first machine learning model 316, the karyotyping by sequencing variant analyzer 320, the second machine learning model 321, and the report generator 325 can be a hardware-based device. For example, a process to cause the second machine learning model 321 to generate a set of significance values for a set of detected variants in the sample or set of samples can be implemented on an IC chip(s).

The data preparation module 310 can receive information from a sample or set of samples from the memory 302 and/or from the set of databases 360. The information from the sample or set of samples can be pre-processed by the data preparation module 310 before training and/or executing the first machine learning model 316 and/or the second machine learning model 321. In some instances, the data preparation module 310 can categorize the information from the sample or set of samples to a set of samples from healthy individuals, a set of clinical samples, a set of research samples, a set of known variant positions, a set of samples with variants of known clinical significance, and/or the like. The data preparation module 310 can scan process the information from the sample or set of samples, for example to align to a reference or a draft genome, or to generate a training contact matrix. Each variant in an information of a sample from the set of samples is known, and is used to label the type of variant.

In some instances, the data preparation module 310 can normalize the sequencing reads or contact matrix from the sample or set of samples to a common format and/or a common scale. For example, the preparation module 310 can normalize a set of images representing the information from the sample or set of samples to a common image size of 256 pixels by 256 pixels and to a common image file format of Tagged Image File Format (TIFF). In some instances, the data preparation module 310 can generate a training data. The training data can be a labeled training data that associated a first category of data from the information from the sample or set of samples with a second category of data from the information from the sample or set of samples. For example, the labeled training data can be a set of clinical samples each associated with a variant from a set of known variants.

The karyotyping by sequencing variant detector 315 receives the training contract matrix from the data preparation module 310, and trains the first machine learning model 316. In some instances, the contact matrix from the information from the sample or set of samples can be used at a mixture of resolutions to train the first machine learning model 316 such as, for example, a convolutional neural network (CNN). The first machine learning model 316 can be executed to identify a presence and a type of variants in a sample. In some instances, the karyotyping by sequencing variant detector 315 can recursively execute the first machine learning model 316, creating increasing resolution contact matrixes between classification steps, to precisely identify structural variants to the desired resolution.

In some embodiments, the karyotyping by sequencing variant analyzer 320, receives information from a set of samples with variants of known clinical significance such as, for example, diagnoses, outcomes, drug/treatment response, metabolic effect, and/or the like, from the data preparation module 310, and trains the second machine learning model 321. Information about samples containing structural variants of known clinical or biological significance are processed, using the data preparation module 310 and/or the karyotyping by sequencing variant analyzer 320, with an Hi-C protocol and aligned to a reference or a draft assembly, resulting in a contact matrix. The information from the set of samples with variants of known clinical significance are used to train the second machine learning model such as, for example, a k-nearest neighbors model (KNN). The second machine learning model 321, can be executed to associate a contact matrix features and/or variants with clinical or biological characteristics and/or clinical significance. The report generator 325 can receive a set of identified variants from the first machine learning model 316 and a set of clinical significance of the identified variants of the second machine learning model 321, and generate a report that presents, via a graphical user interface (GUI), the set of identified variants and/or the set of clinical significance of the identified variants to a user of the variants identification device 301.

In use, the variants identification device 301 can receive, at the data preparation module 310, information from a new set of clinical samples and/or a new set of research samples whose clinical significance is unknown. The data preparation module 310 can categorize the information from new set of clinical samples and/or the new set of research samples and process the new set of clinical samples and/or the new set of research samples, for example by aligning to a reference or a draft genome. The karyotyping by sequencing variant detector 315 recursively uses the first machine learning model 316 (e.g., a CNN model), creating increasing resolution contact matrixes between classification steps, to precisely identify a set of structural variants of the desired resolution. Each structural variant from the set of structural variants are then classified using the second machine learning model 321 (e.g., a KNN model) of the karyotyping by sequencing variant analyzer 320 to predict a set of clinical significance and/or biological significance of the set of structural variants. Lastly, the report generator 325 generates a human-readable reports (e.g., similar to classical karyotype-based cytogenetics reports) from the set of structural variants and/or the set of clinical significance and/or biological significance of the set of structural variants.

In some implementations, the first machine learning model and/or the second machine learning model can include a deep learning model, a gradient descent model, a graph network model, a neural network model, a support vector machine, an export system model, a decision tree model, a logistic regression model, a clustering model, a Markov model, a Monte Carlo model, a likelihood model, and/or the like.

The disclosure provides methods of identifying chromosomal structural variants in a subject comprising: (a) training a first machine learning model to detect at least one region of a first contact matrix comprising at least one chromosomal structural variant; (b) receiving a first contact matrix from a subject by the first machine learning model, wherein the contact matrix is produced by a chromosome conformation analysis technique; (c) applying the first machine learning model to the first contact matrix to identify at least one region of the first contact matrix containing at least one chromosomal structural variant; (d) expressing each chromosomal structural variant identified by the first machine learning model as a bounding box comprising a start and an end in a genome, and a label; (e) training a second machine learning model to relate the at least one chromosomal structural variant to biological information; (f) importing the bounding box and the label of the at least one chromosomal structural variant identified by the first machine learning model into the second machine learning model; and (g) applying the second machine learning model to the bounding box and the label of the at least one chromosomal structural variant identified by the first machine learning classifier, after training the second machine learning model; thereby identifying each chromosomal structural variant of the subject and the biological information related to each chromosomal structural variant. In some embodiments, the method further comprises after step (d) and before step (e): (i) generating an second contact matrix, wherein the second contact matrix comprises the start and end genomic locations of the bounding box, and wherein a resolution of the second contact matrix is finer than a resolution of the first contact matrix; (ii) applying the first machine learning model to the second contact matrix to detect at least one region of the second contact matrix containing the at least one chromosomal structural variant; and (iii) expressing the at least one chromosomal structural variant as a second bounding box comprising a start and an end genomic location of the at least one chromosomal structural variant, and the label, wherein the second bounding box comprises a higher resolution than the bounding box.

In some implementations, the first machine learning model and or the second machine learning model can include a type of a neural network such as, for example, a dense layer neural network, a residual neural network, a convolutional neural network, a recurrent neural network, and/or the like. The neural network model can be configured to include an input layer, an output layer, and a set of hidden layers. The set of hidden layers can further include a set of normalization layers, a set of dense layers, a set of convolutional layers, a set of pooling layers, a set of activation layers, a set of dropout layers, and/or the like. At a training stage, the neural network model can be configured to receive as an input a set of contact matrices, a set of sequencing reads from samples with known variants, for example variants of known clinical significance, simulated sequencing reads corresponding to chromosomal structural variants or wild type chromosomes, and/or the like, in form of a batch of data, as an input vector at the input layer, and generate an output. The neural network model can be iteratively trained based on the input and by comparing the output to variants and variants with significance, to generate a trained neural network model. At a verification stage and/or execution stage, the trained neural network model can then be executed to generate an estimate output that closely anticipates the variants and/or variants with significance of samples and/or contact matrices.

In some implementations, the first machine learning model comprises a convolutional neural network (CNN). CNNs are a class of deep neural networks frequently used to analyze visual imagery. CNNs of the disclosure take an input contact matrix and assign importance (learnable weights and biases) to various aspects/objects in the contact matrix and be able to differentiate between contact matrices from datasets with and without chromosomal structural variants and the type and positions of the variants. In some embodiments, the CNN captures relationships in a contact matrix by the application of a series of convolutional filters of various dimensions, pooling operations, drop-out operations and so forth. The convolutional filters can learn local patterns in the contact matrix. The local patterns identified using the convolutional filters can be translation invariant. For example, a local pattern identified in a first position in a training contact matrix can be identified if appeared at a second position, anywhere, at a testing contact matrix. Furthermore, the convolutional filters can be trained on spatial hierarchies of patterns in the contact matrix to learn highly complex patterns in data. For example, a first convolutional layer of the CNN can be trained on patterns of the contact matrix, whereas a second convolutional layer of the CNN can be trained on patterns of the first convolutional layer of the CNN, and so on.

Exemplary CNN architectures suitable for the methods of the instant disclosure include resnet-50 and RetinaNet.

In some embodiments, the CNN is trained on contact matrices generated from simulated and/or biological samples. In some embodiments, training the CNN comprises: (i) receiving a first training dataset by the CNN, wherein the training dataset comprises contact matrices generated from simulated and/or biological samples; (ii) using transfer learning to apply a pre-trained model to the CNN; and (iii) re-training the CNN with a second training dataset, wherein the second training dataset comprises contact matrices from biological samples. In some embodiments, the first training dataset comprises or consists of contact matrices from subjects that do not have chromosomal structural variants. In alternative embodiments, the first training dataset comprises at least one contract matrix form a subject with a chromosomal structural variant. In further alternative embodiments, the first training dataset comprises contact matrixes comprising a plurality of chromosomal structural variants. In some embodiments, the first training dataset comprises full genome contract matrices and contact matrices comprising or consisting essentially of portions of genomes.

“Transfer learning”, as used herein, refers to a process in machine learning wherein a model developed for a first task is re-used as a starting point for developing a model for a second task. Applying transfer learning saves time and computing power when training neural networks. Methods for applying transfer learning to CNNs will be readily apparent to one of ordinary skill in the art.

In some embodiments, the second machine learning model comprises a recurrent neural network, a sense detector or a k-nearest neighbors model, all of which will be known to a person of ordinary skill in the art.

In some embodiments, the second machine learning model comprises as sense detector. A sense detector, also sometimes referred to as a text classifier or text tagging, is a type of machine learning classifier that is trained, and used, to classify text based on meaning. The sense detector can include a Naïve Bayes model, a Support Vector Machine model, a deep learning model, a convolutional neural network model, a recurrent neural network model, and/or a hybrid system that combine machine learning and rule based systems.

Recurrent neural networks (RNNs) are a class of machine learning models where connections between nodes in the network form a directed graph along a temporal sequence. In effect, loops between the nodes allow information to persist (e.g., memorize) in the network. Thus, RNNs are often highly effective in processing sequential data, time series, classifying time series, and/or processing data where order of data has a significance.

A k-nearest neighbors model is a type of machine learning model that is used to classify and regress data. A k-nearest neighbors model is able to identify what category or categories data belongs in, and also estimate the relationships amongst variables in a dataset. In some embodiments, the k-nearest neighbors model is supervised machine learning model that is trained on a training dataset.

In some embodiments, the sense detector is trained using clinical label data from known chromosomal structural variations, diagnosis data, clinical outcome data, drug or treatment response data or metabolic data. Sources of such data are readily known to persons of ordinary skill in the art.

In some embodiments, the machine learning model is a likelihood model classifier. Likelihood model classifiers are a type of supervised machine learning classifiers, as described in further details hereby.

The disclosure provides methods of training a likelihood model classifier comprising (i) receiving a plurality of sets of reads from healthy subjects into the likelihood model classifier; (i) receiving a plurality of sets of reads corresponding to known chromosomal structural variants into the likelihood model classifier; (iii) representing each known chromosomal structural variant as a bounding rectangle comprising a start and an end location in a genome of the chromosomal structural variant, and a label; (iv) partitioning the sets of reads from (i) and (ii) by genomic location; (v) transforming the partitioned sets of reads from (iv) into a geometric data structure; (vi) modeling a frequency of links between any two genomic locations for each of the sets of reads from (i) and (ii) using a negative binomial distribution model; and (vii) training the negative binomial distribution model to recognize a null distribution from the plurality of sets of reads from healthy subjects, wherein the negative binomial distribution model is trained to recognize a null distribution at the bounding rectangle of each known chromosomal structural variant.

The disclosure provides methods of training a likelihood model classifier comprising (i) receiving a plurality of geometric data structures generated from sets of reads from healthy subjects into the machine learning model; (ii) receiving a plurality of geometric data structures generated from sets of reads corresponding to known chromosomal structural variants into the machine learning model; (iii) representing each known chromosomal structural variant as a bounding rectangle comprising a start location and an end location in a genome of the chromosomal structural variant, and a label; (iv) modeling a frequency of links between any two genomic locations for the sets of reads from (i) and (ii) using a negative binomial distribution model; and (v) training the negative binomial distribution model to recognize a null distribution from the plurality of sets of reads from healthy subjects, wherein the negative binomial distribution model is trained to recognize a null distribution at the bounding rectangle of each known chromosomal structural variant. Processing the sets of reads prior to training the classifier can include, inter alia, mapping the reads to a reference genome, excluding reads that map poorly, and generating a geometric data structure from the sets of reads from healthy subjects, or the sets of reads corresponding to known chromosomal structural variants. Generating the geometric data structure can include (i) partitioning the sets of reads by genomic location; and (ii) transforming the partitioned sets of reads into a geometric data structure.

The likelihood model classifier is trained by importing labeled training data. In some embodiments, the training data comprises a representation of each known chromosomal structural variant as a bounding rectangle comprising a start and an end location in a genome of the chromosomal structural variant, and a label. In some embodiments, the training data comprises a plurality of sets of reads from healthy subjects and a plurality of sets of reads corresponding to known chromosomal structural variants. In some embodiments, the training data comprises a plurality of geometric data structures generated from sets of reads from healthy subjects and a plurality of geometric data structures generated from sets of reads corresponding to known chromosomal structural variants. The sets of reads can be simulated, experimentally determined, or a mixture of both. In some embodiments, the sets of reads from healthy subjects comprise reads corresponding to the genomic locations of each known chromosomal structural variant. This allows the likelihood model classifier to model the distribution of linkage frequencies for the null distribution (no CSV) for all the locations of all known chromosomal structural variants. In some preferred embodiments, the training data comprises sets of reads that are independent and identically distributed. In some embodiments, the imported training data is partitioned by genomic location, and transformed into geometric data structure such as a 2-d k-d tree or a matrix.

In some embodiments, a certain probability distribution in the testing data from the subject is assumed and its required parameters (e.g. probability model) are calculated during the training phase. In some embodiments, the probability model used by the likelihood model classifier is determined by the training data. Exemplary probability models include Bernoulli models, binomial models, negative binomial models, multinomial models, Gaussian models or Poisson distributions.

In some embodiments, the probability model comprises a negative binomial distribution. Negative binomial distributions are advantageous over other models in that it can account for over-dispersion of read count data.

In the learning phase of the likelihood model classifier, the input is the training data and the output is the parameters that are required for the likelihood model classifier. Exemplary parameters include maximum likelihood Estimation (MLE), Bayesian estimation (maximum a posteriori) or optimization of loss criterion.

Following training, the likelihood model classifier is applied to a mapped set of chromosomal conformational capture reads from a subject. In some embodiments, applying the likelihood model classifier comprises fitting the transformed and partitioned test set of reads from the subject to the null model and to an alternate model for each known chromosomal structural variant. In some embodiments, the null model is the distribution of linkage frequencies seen in a subject that does not have a known chromosomal structural variant. In fitting to the null model, the likelihood model classifier identifies known chromosomal structural variants by looking for the absence of the null model, which is the distribution of linkages frequencies between every pair of loci found in a healthy subject, rather than looking for the presence of a known chromosomal structural variant. In some embodiments, fitting the transformed and partitioned test set of reads from the subject to the null model comprises fitting across the entire genome. In some alternative embodiments, the fitting comprises fitting across a portion of the genome corresponding to the bounding rectangle of each known chromosomal or subchromosomal structural variant.

In some embodiments, the methods comprise computing a likelihood ratio of the fit of the transformed and partitioned test set of reads to the null model versus the alternative models for each known chromosomal structural variant. Likelihood ratio tests are statistical tests used for comparing the goodness of fit of two statistical models, a null model (no CSV) and an alternative model (the presence of a known CSV). The test is based on the ratio of likelihoods of the two models, and expresses how many times more likely the data are under one model over the other model. Methods of computing likelihood or log-likelihood ratios, or transformations of these ratios scaled by constant factors, are well known to persons of ordinary skill in the art. In some embodiments, a proximity signal is represented in a matrix, or in rectangular subregions of the matrix can be further subdivided into quadrants about a focal coordinate (x, y). In some embodiments, the data in the matrix is binned. In such embodiments, a theoretical model can be developed to describe the changes in proximity signal expected for various structural variants, including balanced translocations, unbalanced translocations, inversions, insertions, deletions, or other copy number variations. Such theoretical models can include the use of beta, gamma, binomial, negative binomial, bimodal, multimodal, empirically fitted spline, Poisson, Dirichlet, uniform, linear, quadratic, polynomial, exponential, logarithmic, triangle, power law, Bayesian, or other suitable distributions, or any combination thereof, to model proximity signal or the apportionment thereof among regions which would theoretically be on the same chromosome, be on different chromosomes, be on the same chromosome with a given distance or range of distances between them, be on the same chromosome with a given relative arrangement, or have any other theoretical structural arrangement relative to each other. In such embodiments, theoretical models may be trained based on data in a single sample, trained against a multi-sample training set, or tuned using human-configured or fixed parameters. In such embodiments, the likelihood of a given theoretical model being present and centered on the focal coordinate can be calculated by measuring the likelihood of the observed data given the model. In such embodiments, a series of such theoretical models, reflecting the expected proximity signal of various types of structural variations being present, can be tested against observed proximity signal in a given region, and a region can be scanned for possible variant calls at various focal coordinates using maximum likelihood gradient descent, the Nelder-Mead method, the Broyden-Fletcher-Goldfarb-Shanno (BFGS) method, binary search, exhaustive search, entropy minimization techniques, or any other suitable optimization or minimization technique. In such embodiments, multiple theoretical models can be compared to combinations of focal points to identify more than one structural variant in a given region, yielding sets of fitted models that represent specific called variants at specific focal coordinates. In such an embodiment, fitted models may be weighted using Akaike information criterion (AIC), Bayesian information criterion (BIC), deviance information criterion (DIC), or any other suitable information criterion measure, in order to select the most likely combination of focal coordinates and called variants to have produced the observed data, thereby controlling for natural variation, background, or noise in the proximity signal and reducing the possibility of false positive or false negative variant calls. In some embodiments, the subject is determined to have a known chromosomal structural variant when the likelihood ratio for that known chromosomal variant is less than 0.5, 0.45, 0.40, 0.35, 0.30, 0.25, 0.20, 0.15, 0.10, 0.09, 0.08, 0.07, 0.06, 0.05, 0.04, 0.03, 0.02, 0.01, 0.009, 0.008, 0.007, 0.006, 0.005, 0.003, 0.002, 0.001, 0.0009, 0.0008, 0.007, 0.006, 0.005, 0.0004, 0.0003, 0.0002 or 0.0001. In some embodiments, the likelihood ratio is greater than 75%, 80%, 85%, 90%, 95%, 96%, 97, 98%, 99%, 99.1%, 99.2%, 99.3%, 99.4%, 99.5%, 99.6%, 99.7%, 99.8% or 99.9%. In some embodiments, the likelihood ratio is expressed as a log likelihood ratio.

### Image Processing Based Methods

The disclosure provides systems and methods for identifying chromosomal structural variants in a subject using chromosomal conformation data from the subject that is represented as an image.

In some embodiments, the methods comprise (a) receiving a contact matrix, wherein the contact matrix is produced by a chromosome conformation analysis technique applied to a sample from the subject; (b) representing the contact matrix as an image, wherein an intensity of each pixel in the image represents a density of links between two genomic locations in the contact matrix; and (c) applying image processing to the image; thereby detecting chromosomal structural variants in the subject.

In some embodiments, the image is a heat map representation of a contact matrix. For example, each pixel in the heat map represents a cell of the contact matrix, each cell represents a between 5 and 500 kbp contiguous nucleotides of the genome of the subject (a “bin”), and the intensity of each pixels is proportional to the interaction frequency between two loci.

In some embodiments, each pixel represents 5-500 kbp of a genome of the subject.

In some embodiments, each pixel represents 40 kbp of a genome of the subject.

In some embodiments, the image processing comprises (i) applying a global normalization to the image; (ii) applying a first threshold to the image; (iii) identifying sub regions of the image corresponding to chromosome comparisons; (iv) applying a second threshold to each sub region; (v) de-noising each sub region; (vi) applying an edge and/or corner detecting algorithm to the image; (vii) applying at least one filter to remove false positives; and (viii) determining the genomic locations of all chromosomal structural variants in the image.

In some embodiments, applying an edge and/or corner detecting algorithm at (vi) comprises applying the edge and/or corner detecting algorithm to each sub region (i.e., each chromosome comparison).

In some embodiments, the global normalization of (i) comprises fitting a matrix of weights to the image. In some embodiments, each cell in the matrix of weights corresponds to a pixel in the image. In some embodiments, the matrix of weights is generated from a contact matrix generated from a healthy sample, and fitting the matrix of weights comprises subtracting the image from the healthy subject from the image. In some embodiments, pixels within 10-300 kbp of a cis-chromosome diagonal of the image are excluded from the image. The cis-chromosomal diagonal and pixels adjacent thereto in the image represent pairs of loci that are either the same loci, or immediately adjacent to each other in a healthy subject. The cis-chromosomal diagonal and pixels adjacent thereto therefore have high interaction frequencies (and corresponding pixel intensities). In some embodiments, subtracting the matrix of weights from the image minimizes a sum of each row and each column of pixels of the image. In some embodiments, subtracting the matrix of weights from the image minimizes a sum of each row and each column of pixels of the image excluding pixels within 10-300 kbp of the cis-chromosome diagonal of the image.

In some embodiments, the contact matrix from a healthy sample is generated using a simulated set of reads, a theoretical set of reads, or a set of reads experimentally determined from a healthy tissue that does not have a disease or disorder. In some embodiments, the healthy tissue is from one subject or patient. In some embodiments, the healthy tissue is from a plurality of healthy subjects. In some embodiments, the contact matrix from a healthy sample is a reference contact matrix, e.g. an average of many contact matrices from subjects who do not have chromosomal structural variants.

In some embodiments, the methods further comprise calculating a balanced interaction density for each pixel. A balanced interaction density is calculated by normalizing and correcting the interaction density for sequencing coverage, sequence features such as restriction enzyme or other specific motifs, abundance, background signal, noise, or variation. In some embodiments, the global threshold is calculated using the balanced density interaction for each pixel.

In some embodiments, the first threshold comprises a global threshold. A global threshold is a threshold that is applied over the entire image. Global thresholding assumes that the pixel intensity in the image has a bimodal distribution, and that background can be subtracted from one or more objects in the image by a simple operation that compares image values with a threshold value T that separates the two groups of pixels.

In some embodiments, an image or matrix is generated from a sample from tissue comprising a disease, disorder, or other phenotype of interest, and a second image or matrix is generated from sample from healthy tissue that does not comprise the disease, disorder or phenotype. In some embodiments, the sample from the healthy tissue can be from healthy tissue from elsewhere on the body of the same person from which the sample comprising the disease, disorder, or other phenotype is obtained. In some embodiments, the sample from the healthy tissue is from one or more separate healthy individuals, or from one or more theoretical models. When more than one source of data for a given image or matrix is available, the data from multiple sources may be combined using averaging, summing, multiplying, single value decomposition, or other arithmetic or linear algebraic means. In some embodiments, the image or matrix generated from a sample from healthy tissue comprises a reference image or matrix. A third image or matrix can then be generated by subtracting, dividing, or otherwise comparing one image or matrix with another; this resulting image or matrix reflects deviations between the two earlier images or matrix and thus highlights in particular differences between the disease, disorder, or other phenotype tissue and healthy tissue.

In some embodiments, images or matrixes from disease, disorder, or other phenotype tissue, and those from healthy tissue, are not combined, but are preserved as two populations. The populations can be compared using Eigen decomposition, covariance analysis, per-pixel z-score, or other linear algebraic means.

In some embodiments, the edge and/or corner detecting algorithm comprises a Harris corner method, a Roberts cross method, a Hough transform, a derivative calculation, a Scharr filter, a Sobel filter, or other such method known in the art, or a combination thereof.

In some embodiments, the least one filter to remove false positives comprises a Diagonal Path Finder, non-maximum suppression filter, Neighbor threshold, other such method or a combination thereof. Diagonal Path Finder is an iterative algorithm that performs hill climbing up a gradient (such as a Hi-C interaction frequency gradient in a contact matrix or image thereof) and checks to see whether or not it finds the main diagonal of the image, under non-maximum suppression conditions. If Diagonal Path finder encounters the main diagonal, then the call is considered spurious due to variation in the statistical proximity signal (a false positive). This process relies on the expectation that genuine calls will be local maxima located off the main diagonal of the contact matrix or image thereof. The Harris corner method uses a similar technique to identify when it finds two corners that are so close to each other that they are really just the same corner, and it appearing as two points is an artifact.

### Methods of Treatment

Provided herein are methods of treating a subject with a disease or disorder caused by a chromosomal structural variant. The methods comprise identifying a chromosomal structural variant using the systems and methods of the disclosure, associating the identified chromosomal structural variant with relevant biological information using the systems and methods of the disclosure, recommending a course of treatment, and administering the treatment to the subject.

By comprehensively identifying chromosomal structural variants and relating these variants to diseases and disorders and treatment methods, the systems and methods of the disclosure allow clinicians and doctors to tailor treatments to individual subjects. For example, chromosomal structural variants found in some cancers are associated with better or worse clinical outcomes for particular cancer therapies. In one specific example, methods of the disclosure can be used to identify breast cancers with copy number increases in ERBB2 (epidermal growth factor receptor 2, or HER2), which can be targeted with EGFR inhibitors as part of a recommended course of treatment. Further non-limiting examples of targeted cancer therapies are shown in Tables 3 and 4.

TABLE 4
Genes and pathways affected by chromosomal
structural variants and targeted therapies.

| Target | Pathway | Agents |
|---|---|---|
| ERBB2 (HER2) | RAS/Raf/MAPK and | trastuzumab, pertuzumab, |
| | PI3K/Akt | apatinib, afatinib, neratinib |
| EGFR | PI3K/Akt | erlotinib, gefitinib, |
| | | dacomitinib, neratinib, |
| | | simertinib, rociletinib, |
| | | olmutinib |
| FLT3-ITD | STAT, ERK, AKT, C-Myc | sorafenib, daunoribuicin, |
| | | cytarabine |
| VEGF and mTOR | VEGF and mTOR | sorafenib, sunitinib, |
| | | pazopanib, bevacizumab, |
| | | temsirolimus, everolimus |
| VEGFR | Ras/Raf/MEK/ERK | sorafenib, dovitinib, |
| | | Trametinib |
| BCR-Abl | | imatinib, nilotinib, dasatinib, |
| | | bosutinib, ponatinib, |
| | | bafetinib |

Any chromosomal structural variant that causes a disease or disorder falls is envisaged as within scope of the disorder.

Any chromosomal structural variant that causes a disease or disorder with a recommended treatment regimen falls is envisaged as within scope of the disorder.

Recommended treatments, for example for specific cancers associated with or caused by chromosomal structural variants include, but are not limited to, chemotherapy, radiation, small molecules, combination therapies, targeted cancer therapies, immunotherapies and the like.

Chemotherapies include use of alkylating agents such as cyclophosphamide or temozolamide, antimetabolites such as 5-fluorouracil or gemcitabine, anti-tumor antibiotics (doxorubicin, daunorubicin), topoisomerase inhibitors (e.g., etoposide, irinotecan, topotecan), mitotic inhibitors (e.g., docitaxel, paclitaxel, vinblastine), platinum based therapies (e.g., oxaliplatin, carboplatin) or combinations thereof.

Targeted cancer therapies can be targeted to a particular biomarker associated with, or encompassed by, the CSVs identified using the methods herein. Targeted therapies can include administration of small molecules such as tyrosine kinase inhibitors (e.g., imatinib, gefitinib, erlotinib, sorafenib, sunitinib, dasatinib, lapatinib, nilotinib, bortezomib), Janus kinase inhibitors (e.g., tofacitinib), ALK inhibitors (e.g., crizotinib), Bcl-2 inhbitors (e.g., obatoclax, navitoclax), PARP inhibitors (e.g., iniparib, olaparib), PI3K inhibitors (e.g., perifosine), VEGFR2 inhibitors (e.g., Apatinib), Braf inhibitors (e.g., vemurafenib, dabrafenib), MEK inhibitors (e.g., trametinib), CDK inhibitors, Hsp90 inhibitors and serine/threonine kinase inhibitors (e.g., Temsirolimus, Everolimus, Vemurafenib, Trametinib, Dabrafenib).

Immunotherapies can include adoptive cell therapies, such as chimeric antigen receptor (CAR) T cell therapies. Immunotherapies can include antibody therapies, for example the administration of Pembrolizumab, Rituximab, Trastuzumab, Alemtuzumab, Cetuximab, Bevacizumab or Ipilimumab.

### Computer Systems and Software

The methods described herein may be used in the context of a computer system or as part of software or computer-executable instructions that are stored in a computer-readable storage medium.

In some embodiments, a system (e.g., a computer system) may be used to implement certain features of some of the embodiments of the invention. For example, in certain embodiments, a system (e.g., a computer system) for training a machine learning model is provided.

In certain embodiments, the system may include one or more memory and/or storage devices. The memory and storage devices may be one or more computer-readable storage media that may store computer-executable instructions that implement at least portions of the various embodiments of the invention. In one embodiment, the system may include a computer-readable storage medium which stores computer-executable instructions that include, but are not limited to, one or both of the following: (i) instructions for importing a test set of reads from a sample from the subject, wherein the test set of reads is generated by a chromosome conformation analysis technique; (ii) instructions for mapping the test set of reads from the subject onto a reference genome; (iii) instructions for applying a machine learning model to the test set of reads from the subject, wherein the machine learning model is trained to distinguish between sets of reads from healthy subjects and set of reads corresponding to known chromosomal structural variants; (iv) instructions for computing a likelihood that the test set of reads contains a known chromosomal structural variant; and (v) instructions for generating a karyotype of the subject. In an alternative embodiment, the system may include a computer-readable storage medium which stores computer-executable instructions that include, but are not limited to, one or both of the following: (i) instructions for importing a first contact matrix from a subject into a first machine learning model, wherein the first contact matrix is produced by a chromosome conformation analysis technique; (ii) instructions for applying the first machine learning model to the contact matrix to detect at least one region of the first contact matrix comprising at least one chromosomal structural variant; (iii) instructions for expressing each chromosomal structural variant identified by the first machine learning model as a bounding box comprising a start and an end in a genome, and a label; (iv) instructions for importing the bounding box and the label of the at least one chromosomal structural variant identified by the first machine learning model into a second machine learning model; and (v) instructions for applying the second machine learning model, wherein the second machine learning model is trained to relate a chromosomal structural variant to biological information. Such instructions may be carried out in accordance with the methods described in the embodiments above.

In certain embodiments, the system may include a processor configured to perform one or more steps including, but not limited to (i) receiving a set of input files which comprise the test set of reads from the subject and the reference genome; and (ii) executing the computer-executable instructions stored in the computer-readable storage medium. In an alternative embodiment, the system may include a processor configured to perform one or more steps including, but not limited to (i) receiving a set of input files which comprise at least the first contact matrix from the subject and the reference genome; and (ii) executing the computer-executable instructions stored in the computer-readable storage medium. The set of input files may include, but is not limited to, a file that includes a set of reads generated by a chromosome conformation analysis technique (e.g., Hi-C, described above); one or more files that include a reference genome, one or more training datasets for a first machine learning model or second machine learning model comprising experimental or simulated chromosomal conformation capture reads, images generated from chromosomal conformational capture datasets, an experimental chromosome conformational capture dataset derived from a subject for analysis, a list comprising known chromosomal structural variants, and clinical and/or biological information relevant to chromosomal structural variants. The steps may be performed in accordance with the methods described in the embodiments above.

The computer system may be a server computer, a client computer, a personal computer (PC), a user device, a tablet PC, a laptop computer, a personal digital assistant (PDA), a cellular telephone, an iPhone, an iPad, a Blackberry, a processor, a telephone, a web appliance, a network router, switch or bridge, a console, a hand-held console, a (hand-held) gaming device, a music player, any portable, mobile, hand-held device, wearable device, or any machine capable of executing a set of instructions, sequential or otherwise, that specify actions to be taken by that machine.

The computing system may include one or more central processing units (“processors”), memory, input/output devices, e.g. keyboard and pointing devices, touch devices, display devices, storage devices, e.g. disk drives, and network adapters, e.g. network interfaces, that are connected to an interconnect.

According to some aspects, the interconnect is an abstraction that represents any one or more separate physical buses, point-to-point connections, or both, connected by appropriate bridges, adapters, or controllers. The interconnect, therefore, may include, for example a system bus, a peripheral component interconnect (PCI) bus or PCI-Express bus, a HyperTransport or industry standard architecture (ISA) bus, a small computer system interface (SCSI) bus, a universal serial bus (USB), IIC (12C) bus, or an Institute of Electrical and Electronics Engineers (IEEE) standard 1394 bus, also referred to as Firewire®.

In addition, data structures and message structures may be stored or transmitted via a data transmission medium, e.g. a signal on a communications link. Various communications links may be used, e.g. the Internet, a local area network, a wide area network, or a point-to-point dial-up connection. Thus, computer readable media can include computer-readable storage media, e.g. non-transitory media, and computer-readable transmission media.

The instructions stored in memory can be implemented as software and/or firmware to program one or more processors to carry out the actions described above. In some embodiments of the invention, such software or firmware may be initially provided to the processing system by downloading it from a remote system through the computing system, e.g. via the network adapter.

The various embodiments of the invention introduced herein can be implemented by, for example, programmable circuitry, e.g. one or more microprocessors, programmed with software and/or firmware, entirely in special-purpose hardwired, i.e. non-programmable, circuitry, or in a combination of such forms. Special purpose hardwired circuitry may be in the form of, for example, one or more ASICs, PLDs, FPGAS, etc.

Some portions of the detailed description may be presented in terms of algorithms, which may be symbolic representations of operations on data bits within a computer memory. These algorithmic descriptions and representations are those methods used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

The algorithms and displays presented herein are not inherently related to any particular computer or other apparatus. Various general purpose systems may be used with programs in accordance with the teachings herein, or it may prove convenient to construct more specialized apparatus to perform the methods of some embodiments.

Moreover, while embodiments have been described in the context of fully functioning computers and computer systems, those skilled in the art will appreciate that the various embodiments are capable of being distributed as a program product in a variety of forms, and that the disclosure applies equally regardless of the particular type of machine or computer-readable media used to actually effect the distribution.

Further examples of machine-readable storage media, machine-readable media, or computer-readable (storage) media include but are not limited to recordable type media such as volatile and non-volatile memory devices, floppy and other removable disks, hard disk drives, optical disks (e.g., Compact Disk Read-Only Memory (CD ROMS), Digital Versatile Disks, (DVDs), etc.), among others, and transmission type media such as digital and analog communication links.

### ENUMERATED EMBODIMENTS

The invention may be defined by reference to the following enumerated, illustrative embodiments:
1. A method of treating a subject with a chromosomal structural variant comprising:
   a. receiving a test set of reads from a sample from the subject;
   b. aligning the test set of reads from the subject to a reference genome to produce a mapped set of reads from the subject;
   c. training a machine learning model to distinguish between sets of reads from healthy subjects and sets of reads corresponding to known chromosomal structural variants;
   d. applying the machine learning model to the mapped set of reads from the subject after training the machine learning model;
   e. computing a likelihood that the subject has a known chromosomal structural variant based on applying the machine learning model to the mapped set of reads from the subject; and
   f. generating a karyotype of the subject based on the likelihood the subject has the known chromosomal structural variant;
   wherein the test set of reads, the sets of reads from healthy subjects and the sets of reads corresponding to known chromosomal structural variants are generated by a chromosome conformation analysis technique.
2. The method of embodiment 1, wherein the known chromosomal structural variant causes a disease or a disorder in a subject.
3. The method of embodiment 1 or 2, further comprising treating the subject for the disease or disorder caused by the known chromosomal structural if the karyotype indicates that the subject has said known chromosomal structural variant.
4. The method of any one of embodiments 1-3, wherein the machine learning model includes a deep learning model, a gradient descent model, a graph network model, a neural network model, a support vector machine, an export system model, a decision tree model, a logistic regression model, a clustering model, a Markov model, a Monte Carlo model, or a likelihood model.
5. The method of any one of embodiments 1-3, wherein the machine learning model is a likelihood model classifier.
6. The method of embodiment 5, wherein training the likelihood model classifier in step (c) comprises:
   i. receiving a plurality of sets of reads from healthy subjects into the machine learning model;
   ii. importing a plurality of sets of reads corresponding to known chromosomal structural variants into the machine learning model;
   iii. representing each known chromosomal structural variant as a bounding rectangle comprising a start location and an end location in a genome of the chromosomal structural variant, and a label;
   iv. partitioning the sets of reads from (i) and (ii) by genomic location;
   v. transforming the partitioned sets of reads from (iv) into a geometric data structure;
   vi. modeling a frequency of links between any two genomic locations for each of the sets of reads from (i) and (ii) using a negative binomial distribution model; and
   vii training the negative binomial distribution model to recognize a null distribution from the plurality of sets of reads from healthy subjects,
   wherein the negative binomial distribution model is trained to recognize a null distribution at the bounding rectangle of each known chromosomal structural variant.
7. The method of embodiment 6, wherein the geometric data structure represents a frequency of links between any two genomic locations in each of the sets of reads from (i) and (ii).
8. The method of embodiment 6 or 7, wherein the partitioning step (iv) partitions the sets of reads from (i) and (ii) into genomic locations corresponding to cytogenetic bands in a karyotype.
9. The method of embodiment 8, wherein the cytogenetic bands in the karyotype comprise a resolution of about 5 Mb per band.
10. The method of any one of embodiments 6-9, wherein at least one set of reads corresponding to a known chromosomal structural variant in (ii) is experimentally determined.
11. The method of any one of embodiments 6-9, wherein at least one set of reads corresponding to a known chromosomal structural variant in (ii) is simulated.
12. The method of any one of embodiments 6-11, wherein at least one set of reads from healthy subjects in (i) comprises a simulated set of reads, a theoretical set of reads, or a set of reads experimentally determined from a healthy tissue.
13. The method of embodiment 12, wherein the healthy tissue comprises a tissue from the subject that does not have the disease or disorder.
14. The method of any one of embodiments 6-13, wherein the sets of reads from healthy subjects comprise reads corresponding to the genomic locations of each known chromosomal structural variant.
15. The method of any one of embodiments 6-14, wherein the geometric data structure is a k-dimensional tree (k-d tree).
16. The method of embodiment 15, wherein the k-d tree is a 2 dimensional (2-d) k-d tree.
17. The method of embodiment 16, wherein a first axis of the k-d tree represents a first genomic region, and a second axis of the k-d represents a second genomic location, and wherein the k-d tree represents a frequency of links between any two genomic locations in each of the sets of reads from (i) and (ii).
18. The method of any one of embodiments 15-17, wherein the k-d tree can encode an arbitrary resolution.
19. The method of embodiment 18, wherein the arbitrary resolution is chosen based on the size of a known chromosomal structural variant.
20. The method of any one of embodiments 6-14, wherein the geometric data structure is a matrix.
21. The method of embodiment 20, wherein each cell of the contact matrix represents a frequency of links between any two genomic locations in each of the sets of reads from (i) and (ii).
22. The method of embodiment 21, wherein each cell of the matrix comprises between about 1 million and 10 million base pairs (bp) of the genome of the subject.
23. The method of embodiment 21, wherein each cell of the matrix comprises between about 3 million bp of the genome of the subject.
24. The method of any one of embodiments 6-23, wherein the label at step (iii) identifies the known chromosomal structural variant as a balanced translocation, an unbalanced translocation, an inversion, an insertion, a deletion, a repeat expansion, or a combination thereof.
25. The method of any one of embodiments 1-24, further comprising filtering out reads in the test set of reads that align poorly to the reference genome prior to applying the machine learning model.
26. The method of any one of embodiments 1-25, further comprising partitioning the test set of reads from the subject by genomic location and transforming the partitioned test set of reads into a geometric data structure prior to applying the machine learning model.
27. The method of embodiment 26, wherein applying the machine learning model at step (d) comprises fitting the transformed and partitioned test set of reads from the subject to the null model and to an alternate model for each known chromosomal structural variant.
28. The method of embodiment 27, wherein the fitting comprises fitting across the entire genome.
29. The method of embodiment 26, wherein the fitting comprises fitting across a portion of the genome corresponding to the bounding rectangle of each known chromosomal or subchromosomal structural variant.
30. The method of any one of embodiments 6-29, wherein step (e) comprises computing a likelihood ratio of the fit of the transformed and partitioned test set of reads to the null model versus the alternative models for each known chromosomal structural variant.
31. The method of embodiment 30, wherein the subject is determined to have a known chromosomal structural variant when the likelihood ratio for that known chromosomal variant is less than 0.5, 0.45, 0.40, 0.35, 0.30, 0.25, 0.20, 0.15, 0.10, 0.09, 0.08, 0.07, 0.06, 0.05, 0.04, 0.03, 0.02, 0.01, 0.009, 0.008, 0.007, 0.006, 0.005, 0.003, 0.002, 0.001, 0.0009, 0.0008, 0.007, 0.006, 0.005, 0.0004, 0.0003, 0.0002 or 0.0001.
32. The method of embodiment 30, wherein the likelihood ratio is greater than 75%, 80%, 85%, 90%, 95%, 96%, 97, 98%, 99%, 99.1%, 99.2%, 99.3%, 99.4%, 99.5%, 99.6%, 99.7%, 99.8% or 99.9%.
33. The method of embodiment 30, wherein the likelihood ratio is expressed as a log likelihood ratio.
34. The method of any one of embodiments 1-33, wherein the chromatin conformation analysis technique comprises chromatin conformation capture (3C), circularized chromatin conformation capture (4C), carbon copy chromosome conformation capture (5C), chromatin immunoprecipitation (ChIP), ChIP-Loop, Hi-C, combined 3C-ChIP-cloning (6C), Capture-C, Split-pool barcoding (SPLiT-seq), Nuclear Ligation Assay (NLA), Single-cell Hi-C (scHi-C), Combinatorial Single-cell Hi-C, Concatamer Ligation Assay (COLA), Cleavage Under Targets and Release Using Nuclease (CUT& RUN), in vitro proximity ligation (Chicago®), in situ proximity ligation (in situ Hi-C), proximity ligation followed by sequencing on an Oxford Nanopore machine (Pore-C), proximity ligation sequenced on a Pacific Biosciences machine (SMRT-C), DNase Hi-C, Micro-C or Hybrid Capture Hi-C.
35. The method of any one of embodiments 1-34, wherein the subject has cancer.
36. The method of embodiment 35, wherein the sample is from a tumor.
37. The method of embodiment 36, wherein the tumor is a solid tumor or a liquid tumor.
38. A system for determining if a subject has a known chromosomal structural variant comprising:
   a. a computer-readable storage medium which stores computer-executable instructions comprising:
      i. instructions for receiving a test set of reads from a sample from the subject,
         wherein the test set of reads is generated by a chromosome conformation analysis technique;
      ii. instructions for mapping the test set of reads from the subject onto a reference genome;
      iii. instructions for applying a machine learning model to the test set of reads from the subject after training the machine learning model,
   wherein the machine learning model is trained to distinguish between sets of reads from healthy subjects and sets of reads corresponding to known chromosomal structural variants;
      iv. instructions for computing a likelihood that the test set of reads contains a known chromosomal structural variant based on applying the machine learning model to the test set of reads; and
      v. instructions for generating a karyotype of the subject based on the likelihood the subject has the known chromosomal structural variant; and
   b. a processor which is configured to perform steps comprising:
      i. receiving a set of input files which comprise the test set of reads from the subject and the reference genome; and
      ii. executing the computer-executable instructions stored in the computer-readable storage medium.
39. The system of embodiment 38, wherein the computer-executable instructions further comprising instructions for receiving a training data set and instructions for training the machine learning model to distinguish between sets of reads from healthy subjects and sets of reads corresponding to known chromosomal structural variants.
40. The system of embodiment 38 or 39, wherein the processor is further configured to perform the step of training the machine learning model to distinguish between sets of reads from healthy subjects and sets of reads corresponding to known chromosomal structural variants.
41. The system of any one of embodiments 38-40, wherein the known chromosomal structural variants each cause a disease or a disorder in a subject.
42. The system of any one of embodiments 38-41, wherein the machine learning model includes a deep learning model, a gradient descent model, a graph network model, a neural network model, a support vector machine, an export system model, a decision tree model, a logistic regression model, a clustering model, a Markov model, a Monte Carlo model or a likelihood model.
43. The system of any one of embodiments 38-41, wherein the machine learning model is a likelihood model classifier.
44. The system of embodiment 43, wherein training the likelihood model classifier comprises:
   i. receiving a plurality of sets of reads from healthy subjects into the machine learning model;
   ii. receiving a plurality of sets of reads corresponding to known chromosomal structural variants into the machine learning model;
   iii. representing each known chromosomal structural variant as a bounding rectangle comprising a start location and an end location in a genome of the chromosomal structural variant, and a label;
   iv. partitioning the sets of reads from (i) and (ii) by genomic location;
   v. transforming the partitioned sets of reads from (iv) into a geometric data structure;
   vi. modeling a frequency of links between any two genomic locations for each of the sets of reads from (i) and (ii) using a negative binomial distribution model; and
   vii. training the negative binomial distribution model to recognize a null distribution from the plurality of sets of reads from healthy subjects,
   wherein the negative binomial distribution model is trained to recognize a null distribution at the bounding rectangle of each known chromosomal structural variant.
45. The system of embodiment 44, wherein the geometric data structure represents a frequency of links between any two genomic locations in each of the sets of reads from (i) and (ii).
46. The system of embodiment 44 or 45, wherein the partitioning step (iv) partitions the sets of reads from (i) and (ii) into genomic locations corresponding to cytogenetic bands in a karyotype.
47. The system of embodiment 46, wherein the cytogenetic bands in the karyotype comprise a resolution of about 5 Mb per band.
48. The system of any one of embodiments 44-47, wherein at least one set of reads corresponding to a known chromosomal structural variant in (ii) is experimentally determined.
49. The system of any one of embodiments 44-47, wherein at least one set of reads corresponding to a known chromosomal structural variant in (ii) is simulated.
50. The system of any one of embodiments 44-49, wherein at least one set of reads from healthy subjects in (i) comprises a simulated set of reads, a theoretical set of reads or a set of reads experimentally determined from a healthy tissue.
51. The system of embodiment 50, wherein the healthy tissue comprises a tissue from the subject that does not have the disease or disorder.
52. The system of any one of embodiments 44-51, wherein the sets of reads from healthy subjects comprise reads corresponding to the genomic locations of each known chromosomal structural variant.
53. The system of any one of embodiments 44-52, wherein the geometric data structure is a k-dimensional tree (k-d tree).
54. The system of embodiment 53, wherein the k-d tree is a 2 dimensional (2-d) k-d tree.
55. The system of embodiment 54, wherein a first axis of the 2-d k-d tree represents a first genomic region, and a second axis of the k-d represents a second genomic location, and wherein the k-d tree represents a frequency of links between any two genomic locations in each of the sets of reads from (i) and (ii).
56. The system of any one of embodiments 53-55, wherein the 2-d k-d tree can encode an arbitrary resolution.
57. The system of embodiment 56, wherein the arbitrary resolution is chosen based on the size of a known chromosomal structural variant.
58. The system of any one of embodiments 44-52, wherein the geometric data structure is a matrix.
59. The system of embodiment 58, wherein each cell of the matrix represents a frequency of links between any two genomic locations in each of the sets of reads from (i) and (ii).
60. The system of embodiment 59, wherein each cell of the matrix comprises between about 1 million and 10 million bp of the genome of the subject.
61. The system of embodiment 59, wherein each cell of the matrix comprises between about 3 million bp of the genome of the subject.
62. The system of any one of embodiments 44-61, wherein the label at step (iii) identifies the known chromosomal structural variant as a balanced translocation, an unbalanced translocation, an inversion, an insertion, a deletion, a repeat expansion, or a combination thereof.
63. The system of any one of embodiments 39-62, further comprising filtering out reads in the test set of reads that align poorly to the reference genome prior to applying the machine learning model.
64. The system of any one of embodiments 39-63, further comprising partitioning the test set of reads from the subject by genomic location and transforming the partitioned test set of reads into a geometric data structure prior to applying the machine learning model.
65. The system of embodiment 64, wherein applying the machine learning model comprises fitting the transformed and partitioned test set of reads from the subject to the null model and to an alternate model for each known chromosomal structural variant.
66. The system of embodiment 65, wherein the fitting comprises fitting across the entire genome.
67. The system of embodiment 65, wherein the fitting comprises fitting across a portion of the genome corresponding to the bounding rectangle of each known chromosomal or subchromosomal structural variant.
68. The system of any one of embodiments 44-67, wherein computing a likelihood comprises computing a likelihood ratio of the fit of the transformed and partitioned test set of reads to the null model versus the alternative models for each known chromosomal structural variant.
69. The system of embodiment 68, wherein the subject is determined to have a known chromosomal structural variant when the likelihood ratio for that known chromosomal variant is less than 0.5, 0.45, 0.40, 0.35, 0.30, 0.25, 0.20, 0.15, 0.10, 0.09, 0.08, 0.07, 0.06, 0.05, 0.04, 0.03, 0.02, 0.01, 0.009, 0.008, 0.007, 0.006, 0.005, 0.003, 0.002, 0.001, 0.0009, 0.0008, 0.007, 0.006, 0.005, 0.0004, 0.0003, 0.0002 or 0.0001.
70. The system of embodiment 68, wherein the likelihood ratio is greater than 75%, 80%, 85%, 90%, 95%, 96%, 97, 98%, 99%, 99.1%, 99.2%, 99.3%, 99.4%, 99.5%, 99.6%, 99.7%, 99.8% or 99.9%.
71. The system of embodiment 68, wherein the likelihood ratio is expressed as a log likelihood ratio.
72. The system of any one of embodiments 38-71, wherein chromatin conformation analysis technique comprises chromatin conformation capture (3C), circularized chromatin conformation capture (4C), carbon copy chromosome conformation capture (5C), chromatin immunoprecipitation (ChIP), ChIP-Loop, Hi-C, combined 3C-ChIP-cloning (6C), Capture-C, Split-pool barcoding (SPLiT-seq), Nuclear Ligation Assay (NLA), Single-cell Hi-C (scHi-C), Combinatorial Single-cell Hi-C, Concatamer Ligation Assay (COLA), Cleavage Under Targets and Release Using Nuclease (CUT& RUN), in vitro proximity ligation (Chicago®), in situ proximity ligation (in situ Hi-C), proximity ligation followed by sequencing on an Oxford Nanopore machine (Pore-C), proximity ligation sequenced on a Pacific Biosciences machine (SMRT-C), DNase Hi-C, Micro-C or Hybrid Capture Hi-C.
73. The system of any one of embodiments 38-72, wherein the subject has cancer.
74. The system of embodiment 73, wherein the sample is from a tumor.
75. The system of embodiment 74, wherein the tumor is a solid tumor or a liquid tumor.
76. A method of identifying chromosomal structural variants in a subject comprising:
   a. training a first machine learning model to identify at least one region of a first contact matrix comprising at least one chromosomal structural variant;
   b. receiving the first contact matrix from a subject by the first machine learning model,
   wherein the first contact matrix is produced by a chromosome conformation analysis technique;
   c. applying the first machine learning model to the first contact matrix to identify at least one region of the first contact matrix containing at least one chromosomal structural variant;
   d. expressing each chromosomal structural variant identified by the first machine learning model as a bounding box comprising a start location and an end location in a genome, and a label;
   e. training a second machine learning model to relate the at least one chromosomal structural variant to biological information;
   f. receiving the bounding box and the label of the at least one chromosomal structural variant identified by the first machine learning model by the second machine learning model; and
   g. applying the second machine learning model to the bounding box and the label of the at least one chromosomal structural variant identified by the first machine learning classifier, after training the second machine learning model;
   thereby identifying each chromosomal structural variant of the subject and the biological information related to each chromosomal structural variant of the subject.
77. The method of embodiment 76, wherein each cell of the first contact matrix comprises between about 100 bp and 10,000,000 bp of the genome of the subject.
78. The method of embodiment 76 or 77, wherein the first contact matrix comprises the entire genome of the subject.
79. The method of any one of embodiments 76-78, further comprising, after step (d) and before step (e):
   i. generating a second contact matrix,
   wherein the second contact matrix comprises the start and end genomic locations of the bounding box, and
   wherein a resolution of the second contact matrix is finer than a resolution of the first contact matrix;
   ii. applying the first machine learning model to the second contact matrix to identify at least one region of the second contact matrix containing the at least one chromosomal structural variant; and
   iii. expressing the at least one chromosomal structural variant as a second bounding box comprising a second start and a second end genomic location of the at least one chromosomal structural variant, and the label,
   wherein the second bounding box comprises a higher resolution than the bounding box.
80. The method of embodiment 79, further comprising repeating steps (i), (ii) and (iii) until a resolution of at least 500,000 bp per cell, at least 100,000 bp per cell, at least 50,000 bp per cell, at least 10,000 bp per cell, at least 1,000 bp per cell, at least 500 bp per cell or at least 100 bp per cell of the contact matrix is reached.
81. The method of any one of embodiments 76-80, wherein the first contact matrix comprises a data structure that can be accessed at an arbitrary resolution.
82. The method of embodiment 81, wherein the data structure comprises a k-dimensional tree (k-d tree).
83. The method of embodiment 82, wherein the k-d tree is a 2 dimensional (2-d) k-d tree.
84. The method of embodiment 83, wherein a first axis of the 2-d k-d tree represents a first genomic region, and a second axis of the k-d represents a second genomic location, and wherein the k-d tree represents a frequency of links between any two genomic locations.
85. The method of any one of embodiments 82-84, wherein the 2-d k-d tree can encode an arbitrary resolution.
86. The method of embodiment 85, wherein the arbitrary resolution is chosen based on the size of a known chromosomal structural variant.
87. The method of any one of embodiments 76-86, wherein the first contact matrix is an averaged contact matrix, a median contact matrix or a contact matrix with a percentile cut-off.
88. The method of embodiment 87, wherein the averaged contact matrix has a resolution of between 100 bp per cell and 10,000,000 bp per cell.
89. The method of any one of embodiments 76-88, wherein the label identifies the chromosomal structural variant as a balanced translocation, an unbalanced translocation, an inversion, an insertion, a deletion, a repeat expansion or a combination thereof.
90. The method of any one of embodiments 76-89, wherein the first machine learning model comprises a convolutional neural network (CNN).
91. The method of embodiment 90, wherein training the first machine learning model comprises training the CNN on contact matrices generated from simulated and/or biological samples.
92. The method of embodiment 91, wherein training the CNN comprises:
   i. receiving a first training dataset by the CNN, wherein the training dataset comprises contact matrices generated from simulated and/or biological samples;
   ii. using transfer learning to apply a pre-trained model to the CNN; and
   iii. re-training the CNN with a second training dataset,
   wherein the second training dataset comprises or consists of contact matrices from biological samples.
93. The method of embodiment 92, wherein the first training dataset comprises or consists of contact matrices from subjects that do not have chromosomal structural variants.
94. The method of embodiment 92, wherein the first training dataset comprises at least one contract matrix form a subject with a chromosomal structural variant.
95. The method of embodiment 92, wherein the first training dataset comprises contact matrices comprising a plurality of chromosomal structural variants.
96. The method of any one of embodiments 93-95 wherein the first training dataset comprises full genome contract matrices and contact matrices consisting of portions of genomes.
97. The method of any one of embodiments 76-96, wherein the first contact matrix from the subject is generated by:
   a. performing a chromosome conformation analysis technique on a sample from the subject to generate a set of reads;
   b. aligning the set of reads from the subject to a reference genome; and
   c. transforming the aligned set of reads into a contact matrix.
98. The method of embodiment 97, wherein the chromatin conformation analysis technique comprises chromatin conformation capture (3C), circularized chromatin conformation capture (4C), carbon copy chromosome conformation capture (5C), chromatin immunoprecipitation (ChIP), ChIP-Loop, Hi-C, combined 3C-ChIP-cloning (6C), Capture-C, Split-pool barcoding (SPLIT-seq), Nuclear Ligation Assay (NLA), Single-cell Hi-C (scHi-C), Combinatorial Single-cell Hi-C, Concatamer Ligation Assay (COLA), Cleavage Under Targets and Release Using Nuclease (CUT& RUN), in vitro proximity ligation (Chicago®), in situ proximity ligation (in situ Hi-C), proximity ligation followed by sequencing on an Oxford Nanopore machine (Pore-C), proximity ligation sequenced on a Pacific Biosciences machine (SMRT-C), DNase Hi-C, Micro-C or Hybrid Capture Hi-C.
99. The method of embodiment 97 or 98, further comprising filtering out reads from the set of reads from the subject that align poorly to the reference genome prior to transforming the aligned set of reads from the subject into the contact matrix.
100. The method of any one of embodiments 76-99, wherein the second machine learning model comprises a recurrent neural network, a sense detector or a k-nearest neighbors model.
101. The method of embodiment 100, wherein the sense detector is trained using clinical label data from known chromosomal structural variations, diagnosis data, clinical outcome data, drug or treatment response data or metabolic data.
102. The method of any one of embodiments 76-101, wherein the second machine learning model identifies the chromosomal structural variant as a balanced translocation, an unbalanced translocation, an inversion, an insertion, a deletion, a repeat expansion or a combination thereof
103. The method of any one of embodiments 76-102 wherein the biological information comprises one or more genes, a diagnosis, a patient outcome, a metabolic effect, a drug target, a drug response, a course of treatment or a combination thereof.
104. The method of embodiment 103, wherein the subject has a disease or a disorder caused by the at least one chromosomal structural variant.
105. The method of embodiment 104, wherein the method comprises treating the subject for the disease or disorder caused by the at least one chromosomal structural variant.
106. The method of any one of embodiments 76-105, wherein the subject has cancer.
107. The method of embodiment 106, wherein the first contact matrix from the subject is from a cancer sample.
108. The method of embodiment 107, wherein the cancer is a solid tumor or a liquid tumor.
109. A system for identifying chromosomal structural variants in a subject comprising:
   a. a computer-readable storage medium which stores computer-executable instructions comprising:
      i. instructions for receiving a first contact matrix from a subject by a first machine learning model,
   wherein the first contact matrix is produced by a chromosome conformation analysis technique;
      ii. instructions for applying the first machine learning model to the contact matrix to identify at least one region of the first contact matrix comprising at least one chromosomal structural variant;
      iii. instructions for expressing each chromosomal structural variant identified by the first machine learning model as a bounding box comprising a start and an end in a genome, and a label;
      iv. instructions for receiving the bounding box and the label of the at least one chromosomal structural variant identified by the first machine learning model into a second machine learning model; and
      v. instructions for applying the second machine learning model, wherein the second machine learning model is trained to relate a chromosomal structural variant to biological information, and wherein applying the second machine learning model occurs after training the second machine learning model; and
   b. a processor which is configured to perform steps comprising:
      i. receiving a set of input files which comprise at least the first contact matrix from the subject; and
      ii. executing the computer-executable instructions stored in the computer-readable storage medium.
110. The system of embodiment 109, wherein the computer-executable instructions further comprise instructions for training a first machine learning model to detect at least one region of a contact matrix containing a chromosomal structural variant.
111. The system of embodiment 110, wherein the set of input files further comprises a first training dataset for the first machine learning model.
112. The system of any one of embodiments 109-111, wherein the computer-executable instructions further comprise instructions for training a second machine learning model to relate a chromosomal structural variant to known biological information.
113. The system of embodiment 112, wherein the set of input files further comprises a second training dataset for the second machine learning model.
114. The system of any one of embodiments 101-114, wherein each cell of the first contact matrix comprises between about 100 bp and 10,000,000 bp of the genome of the subject.
115. The system of any one of embodiments 109-114, wherein the first contact matrix comprises the entire genome of the subject.
116. The system of any one of embodiments 109-115, further comprising, after step (d) and before step (e):
   i. generating a second contact matrix, wherein the second contact matrix comprises the start and end genomic locations of the bounding box, and
   wherein a resolution of the second contact matrix is finer than a resolution of the first contact matrix;
   ii. applying the first machine learning model to the second contact matrix to identify at least one region of the second contact matrix containing the at least one chromosomal structural variant; and
   iii. expressing the at least one chromosomal structural variant as a second bounding box comprising a second start and a second end genomic location of the at least one chromosomal structural variant, and the label,
   wherein the second bounding box comprises a higher resolution than the bounding box.
117. The system of embodiment 116, further comprising repeating steps (i), (ii) and (iii) until a resolution of at least 500,000 bp per cell, at least 100,000 bp per cell, at least 50,000 bp per cell, at least 10,000 bp per cell, at least 1,000 bp per cell, at least 500 bp per cell or at least 100 bp per cell of the contact matrix is reached.
118. The system of any one of embodiments 109-117, wherein the first contact matrix comprises a data structure that can be accessed at an arbitrary resolution.
119. The system of embodiment 118, wherein the data structure comprises a k-dimensional tree (k-d tree).
120. The system of embodiment 119, wherein the k-d tree is a 2 dimensional (2-d) k-d tree.
121. The system of embodiment 120, wherein a first axis of the 2-d k-d tree represents a first genomic region, and a second axis of the k-d represents a second genomic location, and wherein the k-d tree represents a frequency of links between any two genomic locations.
122. The system of any one of embodiments 119-121, wherein the 2-d k-d tree can encode an arbitrary resolution.
123. The system of embodiment 122, wherein the arbitrary resolution is chosen based on the size of a known chromosomal structural variant.
124. The system of any one of embodiments 109-123, wherein the first contact matrix is an averaged contact matrix, a median contact matrix or a contact matrix with a percentile cut-off.
125. The system of embodiment 124, wherein the averaged contact matrix has a resolution of between 100 bp per cell and 10,000,000 bp per cell.
126. The system of any one of embodiments 109-125, wherein the label identifies the chromosomal structural variant as a balanced translocation, an unbalanced translocation, an inversion, an insertion, a deletion, a repeat expansion or a combination thereof.
127. The system of any one of embodiments 109-126, wherein the first machine learning model comprises a convolutional neural network (CNN).
128. The system of embodiment 127, wherein training the first machine learning model comprises training the CNN on contact matrices generated from simulated and/or biological samples.
129 The system of embodiment 128, wherein training the CNN comprises:
   i. receiving a first training dataset by the CNN, wherein the training dataset comprises contact matrices generated from simulated and/or biological samples;
   ii. using transfer learning to apply a pre-trained model to the CNN; and
   iii. re-training the CNN with a second training dataset, wherein the second training dataset comprises or consists of contact matrices from biological samples.
130. The system of embodiment 129, wherein the first training dataset comprises or consists of contact matrices from subjects that do not have chromosomal structural variants.
131. The system of embodiment 129, wherein the first training dataset comprises at least one contract matrix form a subject with a chromosomal structural variant.
132. The system of embodiment 129, wherein the first training dataset comprises contact matrixes comprising a plurality of chromosomal structural variants.
133. The system of any one of embodiments 129-131, wherein the first training dataset comprises full genome contract matrices and contact matrices consisting of portions of genomes.
134. The system of any one of embodiments 109-133, wherein the first contact matrix from the subject is generated by:
   a. performing a chromosome conformation analysis technique on a sample from the subject to generate a set of reads;
   b. aligning the set of reads from the subject to a reference genome; and
   c. transforming the aligned set of reads into a contact matrix.
135. The system of embodiment 134, wherein the chromatin conformation analysis technique comprises chromatin conformation capture (3C), circularized chromatin conformation capture (4C), carbon copy chromosome conformation capture (5C), chromatin immunoprecipitation (ChIP), ChIP-Loop, Hi-C, combined 3C-ChIP-cloning (6C), Capture-C, Split-pool barcoding (SPLiT-seq), Nuclear Ligation Assay (NLA), Single-cell Hi-C (scHi-C), Combinatorial Single-cell Hi-C, Concatamer Ligation Assay (COLA), Cleavage Under Targets and Release Using Nuclease (CUT& RUN), in vitro proximity ligation (Chicago®), in situ proximity ligation (in situ Hi-C), proximity ligation followed by sequencing on an Oxford Nanopore machine (Pore-C), proximity ligation sequenced on a Pacific Biosciences machine (SMRT-C), DNase Hi-C, Micro-C or Hybrid Capture Hi-C.
136. The system of embodiment 134 or 135, further comprising filtering out reads from the set of reads from the subject that align poorly to the reference genome prior to transforming the aligned set of reads from the subject into the contact matrix.
137. The system of any one of embodiments 109-136, wherein the second machine learning model comprises a recurrent neural network or a sense detector.
138. The system of embodiment 137, wherein the sense detector is trained using clinical label data from known chromosomal structural variations.
139. The system of any one of embodiments 109-136, wherein the second machine learning model identifies the chromosomal structural variant as a balanced translocation, an unbalanced translocation, an inversion, an insertion, a deletion, a repeat expansion, or a combination thereof
140. The system of any one of embodiments 109-139, wherein the biological information comprises one or more genes, a diagnosis, a patient outcome, a metabolic effect, a drug target, a drug response, a course of treatment or a combination thereof.
141. The system of embodiment 140, wherein the subject has a disease or a disorder caused by the at least one chromosomal structural variant.
142. The system of any one of embodiments 109-141, wherein the subject has cancer.
143. The system of embodiment 1441, wherein the first contact matrix from the subject is from a cancer sample.
144. The system of embodiment 143, wherein the cancer is a solid tumor or a liquid tumor.
145. A method of identifying chromosomal structural variants in a subject comprising:
   a. receiving a contact matrix, wherein the contact matrix is produced by a chromosome conformation analysis technique applied to a sample from the subject;
   b. representing the contact matrix as an image, wherein an intensity of each pixel in the image represents a density of links between two genomic locations in the contact matrix; and
   c. applying image processing to the image;
   thereby detecting chromosomal structural variants in the subject.
146. The method of embodiment 145, wherein each pixel represents 5-500 kilobase pairs (kbp) of a genome of the subject.
147. The method of embodiment 145, wherein each pixel represents 40 kbp of a genome of the subject.
148. The method of any one of embodiments 145-147, wherein the image processing in step (c) comprises:
   i. applying a global normalization to the image;
   ii. applying a first threshold to the image;
   iii. identifying sub regions of the image corresponding to chromosome comparisons;
   iv. applying a second threshold to each sub region;
   v. de-noising each sub region;
   vi. applying an edge and/or corner detecting algorithm to the image;
   vii. applying at least one filter to remove false positives; and
   viii. determining the genomic locations of all chromosomal structural variants in the image.
149. The method of embodiment 148, wherein applying an edge and/or corner detecting algorithm at (vi) comprises applying the edge and/or corner detecting algorithm to each sub region.
150. The method of embodiment 148, wherein the global normalization of (i) comprises fitting a matrix of weights to the image.
151. The method of embodiment 148, wherein each cell in the matrix corresponds to a pixel in the image.
152. The method of embodiment 151, wherein fitting a matrix of weights comprises
   i. generating a contact matrix from a healthy sample;
   ii. representing the contact matrix from the healthy subject as an image from a healthy subject; and
   iii. subtracting the image from the healthy subject from the image,
   wherein pixels within 10-300 kbp of a cis-chromosome diagonal of the image are excluded.
153. The method of embodiment 152, wherein the contact matrix from a healthy sample is generated using a simulated set of reads, a theoretical set of reads or a set of reads experimentally determined from a healthy tissue.
154. The method of embodiment 153, wherein the healthy tissue comprises a tissue from the subject that does not have a disease or disorder.
155. The method of embodiment 153, wherein the contact matrix from the healthy sample comprises a reference matrix.
156. The method of embodiment 152, wherein subtracting the matrix of weights from the image minimizes a sum of each row and each column of pixels of the image.
157. The method of any one of embodiments 148-156, further comprising calculating a balanced interaction density for each pixel.
158. The method of any one of embodiments 148-157, wherein the first threshold comprises a global threshold.
159. The method of embodiment 158, wherein the global threshold is calculated using the balanced density interaction for each pixel.
160. The method of any one of embodiments 148-159, wherein the edge and/or corner detecting algorithm comprises a Harris corner method, a Roberts cross method, a Hough transform or a combination thereof.
161. The method of any one of 148-160, wherein the least one filter to remove false positives comprises a Diagonal Path Finder, non-maximum suppression filter, Neighbor threshold or a combination thereof.
162. The method of any one of embodiments 145-161, wherein the chromosomal structural variant is a balanced translocation, an unbalanced translocation, an inversion, an insertion, a deletion, a repeat expansion or a combination thereof.
163. The method of any one of any one of embodiments 145-162, wherein the subject has a disease or disorder caused by the chromosomal structural variant.
164. The method of embodiment 163, further comprising treating the subject for the disease or disorder caused by the chromosomal structural variant.
165. The method of any one of any one of embodiments 145-164, wherein the chromosome conformation analysis technique chromatin conformation capture (3C), circularized chromatin conformation capture (4C), carbon copy chromosome conformation capture (5C), chromatin immunoprecipitation (ChIP), ChIP-Loop, Hi-C, combined 3C-ChIP-cloning (6C),Capture-C, Split-pool barcoding (SPLIT-seq), Nuclear Ligation Assay (NLA), Single-cell Hi-C (scHi-C), Combinatorial Single-cell Hi-C, Concatamer Ligation Assay (COLA), Cleavage Under Targets and Release Using Nuclease (CUT& RUN), in vitro proximity ligation (Chicago®), in situ proximity ligation (in situ Hi-C), proximity ligation followed by sequencing on an Oxford Nanopore machine (Pore-C), proximity ligation sequenced on a Pacific Biosciences machine (SMRT-C), DNase Hi-C, Micro-C or Hybrid Capture Hi-C.
166. The method of any one of embodiments 145-165, wherein the subject has cancer.
167. The method of embodiment 166, wherein the sample is from a tumor.
168. The method of embodiment 167, wherein the tumor is a solid tumor or a liquid tumor.
169. A system for identifying chromosomal structural variants in a subject, wherein the system is configured to apply the methods of any one of embodiments 145-165.
170. A system for identifying chromosomal structural variants in a subject comprising:
   a. a computer-readable storage medium which stores computer-executable instructions comprising:
      i. instructions for receiving a contact matrix, wherein the contact matrix is produced by a chromosome conformation analysis technique applied to a sample from the subject; ii. instructions for representing the contact matrix as an image, wherein an intensity of each pixel in the image represents a density of links between two genomic locations in the contact matrix; and
      iii. instructions for applying image processing to the image; and
   b. a processor which is configured to perform the steps of executing the computer executable-instructions for receiving a first contact matrix, representing the contact matrix as an image, and applying image processing to the image, which are stored in the computer-readable storage medium;
   thereby detecting chromosomal structural variants in the subject.
171. A method comprising:
   a. contacting a sample from a subject with a stabilizing agent, wherein said sample comprises nucleic acids;
   b. cleaving the nucleic acids into a plurality of fragments comprising at least a first segment and a second segment;
   c. attaching the first segment and the second segment at a junction to generate a plurality of fragments comprising attached segments;
   d. obtaining at least some sequence on each side of the junction of the plurality of fragments comprising attached segments to generate a plurality of reads; and
   e. applying the method of any one of embodiments 1-38, 76-108 or 145-168.
172. The method of embodiment 171, wherein the nucleic acids comprise genomic DNA.
173. The method of embodiment 172, wherein the stabilizing agent comprises ultraviolet light or a chemical fixative.
174. The method of embodiment 173, wherein the chemical fixative comprises formaldehyde.
175. The method of any one of embodiments 171-174, wherein cleaving the nucleic acids comprises mechanical cleavage or enzymatic cleavage.
176. The method of any one of embodiments 171-175, wherein attaching the first segment and the second segment comprises ligation.
177. The method of any one of embodiments 171-176, wherein obtaining at least some sequence on each side of the junction comprises high throughput sequencing.
178. A method of treating a subject with a chromosomal structural variant comprising:
   a. receiving a test set of reads from a sample from the subject;
   b. aligning the test set of reads from the subject to a reference genome to produce a mapped set of reads from the subject;
   c. generating a geometric data structure from the mapped set of reads;
   d. training a machine learning model to distinguish between geometric data structures from sets of reads from healthy subjects and sets of reads corresponding to known chromosomal structural variants;
   e. applying the machine learning model to the geometric data structure from the subject after training the machine learning model;
   f. computing a likelihood that the subject has a known chromosomal structural variant based on applying the machine learning model to the geometric data structure from the subject; and
   g. generating a karyotype of the subject based on the likelihood the subject has the known chromosomal structural variant;
   wherein the test set of reads, the sets of reads from healthy subjects and the sets of reads corresponding to known chromosomal structural variants are generated by a chromosome conformation analysis technique.
179. The method of embodiment 178, wherein the known chromosomal structural variant causes a disease or a disorder in a subject.
180. The method of embodiment 178 or 179, further comprising treating the subject for the disease or disorder caused by the known chromosomal structural if the karyotype indicates that the subject has said known chromosomal structural variant.
181. The method of any one of embodiments 178-180, wherein the machine learning model includes a deep learning model, a gradient descent model, a graph network model, a neural network model, a support vector machine, an export system model, a decision tree model, a logistic regression model, a clustering model, a Markov model, a Monte Carlo model, or a likelihood model.
182. The method of any one of embodiments 178-180, wherein the machine learning model is a likelihood model classifier.
183. The method of embodiment 182, wherein training the likelihood model classifier in step (c) comprises:
   i. receiving a plurality of geometric data structures generated from sets of reads from healthy subjects into the machine learning model;
   ii. receiving a plurality of geometric data structures generated from sets of reads corresponding to known chromosomal structural variants into the machine learning model;
   iii. representing each known chromosomal structural variant as a bounding rectangle comprising a start location and an end location in a genome of the chromosomal structural variant, and a label;
   iv. modeling a frequency of links between any two genomic locations for the sets of reads from (i) and (ii) using a negative binomial distribution model; and
   v. training the negative binomial distribution model to recognize a null distribution from the plurality of sets of reads from healthy subjects,
   wherein the negative binomial distribution model is trained to recognize a null distribution at the bounding rectangle of each known chromosomal structural variant.
184. The method of any one of embodiments 178-183, wherein generating the geometric data structure from the test set of reads, the sets of reads from healthy subjects, or the sets of reads corresponding to known chromosomal structural variants comprises:
   i. partitioning the sets of reads by genomic location; and
   ii. transforming the partitioned sets of reads into a geometric data structure.
185. The method of embodiment 183 or 184, wherein the geometric data structure represents a frequency of links between any two genomic locations in each of sets of reads.
186. The method of embodiment 184 or 185, wherein the partitioning step partitions the set of reads into genomic locations corresponding to cytogenetic bands in a karyotype.
187. The method of embodiment 186, wherein the cytogenetic bands in the karyotype comprise a resolution of about 5 Mb per band.
188. The method of any one of embodiments 183-187, wherein at least one set of reads corresponding to a known chromosomal structural variant in (ii) is experimentally determined.
189. The method of any one of embodiments 183-187, wherein at least one set of reads corresponding to a known chromosomal structural variant in (ii) is simulated.
190. The method of any one of embodiments 183-188, wherein at least one set of reads from healthy subjects in (i) comprises a simulated set of reads, a theoretical set of reads, or a set of reads experimentally determined from a healthy tissue.
191. The method of embodiment 190, wherein the healthy tissue comprises a tissue from the subject that does not have the disease or disorder.
192. The method of any one of embodiments 183-191, wherein the sets of reads from healthy subjects comprise reads corresponding to the genomic locations of each known chromosomal structural variant.
193. The method of any one of embodiments 183-192, wherein the geometric data structure is a k-dimensional tree (k-d tree).
194. The method of embodiment 193, wherein the k-d tree is a 2 dimensional (2-d) k-d tree.
195. The method of embodiment 193, wherein a first axis of the k-d tree represents a first genomic region, and a second axis of the k-d represents a second genomic location, and wherein the k-d tree represents a frequency of links between any two genomic locations in the set of reads from the subject, the sets of reads from healthy subjects or the sets of reads corresponding to known chromosomal structural variants.
196. The method of any one of embodiments 193-195, wherein the k-d tree can encode an arbitrary resolution.
197. The method of embodiment 196, wherein the arbitrary resolution is chosen based on the size of a known chromosomal structural variant.
198. The method of any one of embodiments 178-192, wherein the geometric data structure is a matrix.
199. The method of embodiment 198, wherein each cell of the matrix represents a frequency of links between any two genomic locations in each of the sets of reads from the subject, the sets of reads from healthy subjects or the sets of reads corresponding to known chromosomal structural variants.
200. The method of embodiment 199, wherein each cell of the matrix comprises between about 1 million and 10 million base pairs (bp) of the genome of the subject.
201. The method of embodiment 199, wherein each cell of the matrix comprises between about 3 million bp of the genome of the subject.
202. The method of any one of embodiments 183-201, wherein the label at step (iii) identifies the known chromosomal structural variant as a balanced translocation, an unbalanced translocation, an inversion, an insertion, a deletion, a repeat expansion, or a combination thereof.
203. The method of any one of embodiments 178-202, further comprising filtering out reads in the test set of reads that align poorly to the reference genome prior to applying the machine learning model.
204. The method of embodiment 203, wherein applying the machine learning model at step (e) comprises fitting the geometric data structure from the test set of reads from the subject to the null model and to an alternate model for each known chromosomal structural variant.
205. The method of embodiment 204, wherein the fitting comprises fitting across the entire genome.
206. The method of embodiment 204, wherein the fitting comprises fitting across a portion of the genome corresponding to the bounding rectangle of each known chromosomal or subchromosomal structural variant.
207. The method of any one of embodiments 183-206, wherein step (f) comprises computing a likelihood ratio of the fit of the transformed and partitioned test set of reads to the null model versus the alternative models for each known chromosomal structural variant.
208. The method of embodiment 207, wherein the subject is determined to have a known chromosomal structural variant when the likelihood ratio for that known chromosomal variant is less than 0.5, 0.45, 0.40, 0.35, 0.30, 0.25, 0.20, 0.15, 0.10, 0.09, 0.08, 0.07, 0.06, 0.05, 0.04, 0.03, 0.02, 0.01, 0.009, 0.008, 0.007, 0.006, 0.005, 0.003, 0.002, 0.001, 0.0009, 0.0008, 0.007, 0.006, 0.005, 0.0004, 0.0003, 0.0002 or 0.0001.
209. The method of embodiment 207, wherein the likelihood ratio is greater than 75%, 80%, 85%, 90%, 95%, 96%, 97, 98%, 99%, 99.1%, 99.2%, 99.3%, 99.4%, 99.5%, 99.6%, 99.7%, 99.8% or 99.9%.
210. The method of embodiment 209, wherein the likelihood ratio is expressed as a log likelihood ratio.
211. The method of any one of embodiments 178-210, wherein the chromatin conformation analysis technique comprises chromatin conformation capture (3C), circularized chromatin conformation capture (4C), carbon copy chromosome conformation capture (5C), chromatin immunoprecipitation (ChIP), ChIP-Loop, Hi-C, combined 3C-ChIP-cloning (6C), Capture-C, Split-pool barcoding (SPLIT-seq), Nuclear Ligation Assay (NLA), Single-cell Hi-C (scHi-C), Combinatorial Single-cell Hi-C, Concatamer Ligation Assay (COLA), Cleavage Under Targets and Release Using Nuclease (CUT& RUN), in vitro proximity ligation (Chicago®), in situ proximity ligation (in situ Hi-C), proximity ligation followed by sequencing on an Oxford Nanopore machine (Pore-C), proximity ligation sequenced on a Pacific Biosciences machine (SMRT-C), DNase Hi-C, Micro-C or Hybrid Capture Hi-C.
212. The method of any one of embodiments 178-211, wherein the subject has cancer.
213. The method of embodiment 212, wherein the sample is from a tumor.
214. The method of embodiment 213, wherein the tumor is a solid tumor or a liquid tumor.
215. A system for determining that a subject has a chromosomal structural variant, wherein the system is configured to apply the methods of any one of embodiments 178-214.
216. A system for determining if a subject has a known chromosomal structural variant comprising:
   a. a computer-readable storage medium which stores computer-executable instructions comprising:
      i. instructions for receiving a test set of reads from a sample from the subject, wherein the test set of reads is generated by a chromosome conformation analysis technique;
      ii. instructions for mapping the test set of reads from the subject onto a reference genome;
      iii. instructions for generating a geometric data structure from the mapped set of reads;
      iv. instructions for applying a machine learning model to the geometric data structure from test set of reads from the subject after training the machine learning model,
   wherein the machine learning model is trained to distinguish between geometric data structures sets of reads from healthy subjects and sets of reads corresponding to known chromosomal structural variants;
      v. instructions for computing a likelihood that the geometric data structure from test set of reads contains a known chromosomal structural variant based on applying the machine learning model to the test set of reads; and
      vi. instructions for generating a karyotype of the subject based on the likelihood the subject has the known chromosomal structural variant; and
   b. a processor which is configured to perform steps comprising:
      ii. receiving a set of input files which comprise the test set of reads from the subject and the reference genome; and
   ii. executing the computer-executable instructions stored in the computer-readable storage medium.

The following examples are intended to illustrate various embodiments of the invention. As such, the specific embodiments discussed are not to be construed as limitations on the scope of the invention. It will be apparent to one skilled in the art that various equivalents, changes, and modifications may be made without departing from the scope of invention, and it is understood that such equivalent embodiments are to be included herein. Further, all references cited in the disclosure are hereby incorporated by reference in their entirety, as if fully set forth herein.

### EXAMPLES

### Example 1: Genotype Human Structural Variants of Known Significance

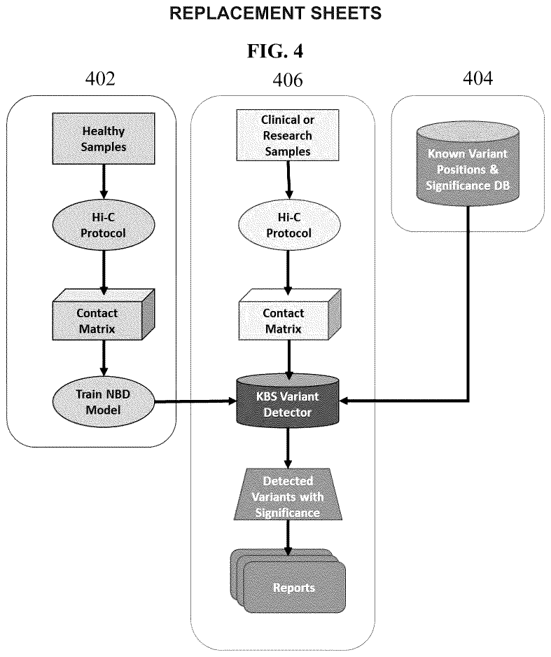

In one implementation (FIG. 4), a likelihood model classifier is created and used to identify variants of known clinical significance in human samples. The likelihood model classifier is trained using Hi-C data derived from both simulated and biological samples, reflecting structural variation present in the sample. Variants are detected with the likelihood model classifier by providing Hi-C data from clinical or research samples outside the training set. The likelihood model classifier represents all variants as bounding rectangles encoding the start and end position (in genomic bands) of the structural variant, with a label. The label can describe the nature of the variant such as balanced or unbalanced translocation, inversion, or insertion, deletion, or repeat expansion. A list of variants with known clinical significance is also input into the likelihood model classifier, with the entire set of all clinically relevant events curated into a database. The Hi-C data is binned into cytogenetic bands and transformed into a geometric data structure (e.g. a KD-Tree) that can be rapidly queried to quantify the number of links between any two genomic regions.

To recursively build the KD-Tree, the following function in C is used. The function calls qsort to sort the kd_nodes on alternating dimensions in with a O(n log n) runtime for each call. The range of the data that is sorted is logged every iteration. The function takes an array header pointer [t] and builds a 2D KD-Tree. The function takes the following parameters, defined as follows: t—a kd_node; start—index of the kd_node array; end—the length of the kd_node array; dim—the dimension 0==x; 1==y. The return statement is the root of the 2D KD tree. Once the KD-Tree is built, “qsort” is used to sort along the dimensions, narrowing the range. The midpoint of the array is calculated using the “mid”. Lastly, if there are nodes left, then more subtrees are built.

The KD-Tree is recursively built as follows:

| | |
|---|---|
| kd_node * make_tree | (kd_node * t, |
| | int start, |
| | int end, |
| | int dim) { |

```
  if (start = = end) return NULL;
qsort (&t[start], end-start, sizeof (kd_node), (dim = = 0 ? cmp_x :
cmp_y));
int mid = start + ((end-start)/2);
if (end-start) > 1 {
  t[mid] .left = make_tree (t, start, mid, (dim+1) % MAX_DIM);
  t[mid] .right = make_tree (t, mid+1, end, (dim+1) % MAX_DIM);
  }
Return &t[mid];
}
```

The KD-Tree can be rapidly queried to quantify the number of links between any two genomic regions. The C function used to recursively query the KD-Tree to find the number of Hi-C links between two loci is described below. This function's runtime complexity is O(sqrt(n)+K), where n is the number of nodes in the tree and K is the number of reported nodes (i.e., nodes with links). This function queries a bounding box X_0, X_1, y_0, y_1 and returns the number of datum within the specified range. The function takes the following parameters, defined as follows: node—kd_node*root of the tree; range—an array pointer of uint32_t for which you wish to query; dim—the starting dimension; c—the count. The function returns 1 is the query is valid, and returns 0 otherwise. The “contained” function checks that the query is within the bounding box. The search is then pruned down to <o(n). Ranges to the left and right of the node are searched. The range is also contained so both nodes are searched.

```
        The KD-Tree is queried as follows:
int query (kd_node * node,
          uint32_t * range,
          int dim,
          uint32_t * c) {
  if (node = = NULL) return 0;
  if (contained(node, range)) {
        *c +=1;
  }
  int i1 = dim + dim;
  int i2 = dim+1 + dim;
  if (node->x[dim] < range [i1]
    && node->x[dim] < range[i2] ) {
    query (node->right, range, (dim+1) % MAX_DIM, c);
  }
  else {
      if (node->x[dim] <>range [i1]
        && node->x[dim] > range[i2] ) {
          query (node-> left, range, (dim+1) % MAX_DIM, c);
        }
        else {
          query (node->left, range, (dim+1) % MAX_DIM, c );
          query (node->right, range, (dim+1) % MAX_DIM, c );
        }
  }
  return 1;
}
```

To accurately test for each possible known variant, the frequency of Hi-C interactions is modeled in training data for that variant using a negative binomial distribution. A negative binomial, unlike the Poisson distribution, can account for over dispersion of the count data. For each variant of known significance's bounding box, the model is trained across a number of healthy control samples, thus learning the null distribution. In clinical or research samples being tested with the model, Hi-C data is generated and mapped, then compute a Likelihood Ratio Test (LRT) for each variant of known significance, with two degrees of freedom. This ratio is applied to determine the chance that each event is real and present in the sample or not.

The results of this method are summarized in a report, such as PDF booklet, that will be returned the user. Importantly, the data and visualizations in the report will include information similar to that in a standard karyotype or FISH report that genetic counselors and clinicians typically see, even though they were not generated with those methods.

The steps below summarize the procedure for the first major KBS application:
1. Map the Hi-C data to the human reference genome (using BWA-mem).
2. Filter out low-quality alignment data (<MQ 20).
3. Transform the hi-c genomic positions into a KD-Tree.
4. Fit the likelihood ratio model.
5. Test new samples for statistical significance.
6. Generate reports.

### Example 2: Detecting and Annotating all Structural Variants in an Organism Using a Convolutional Neural Network (CNN)

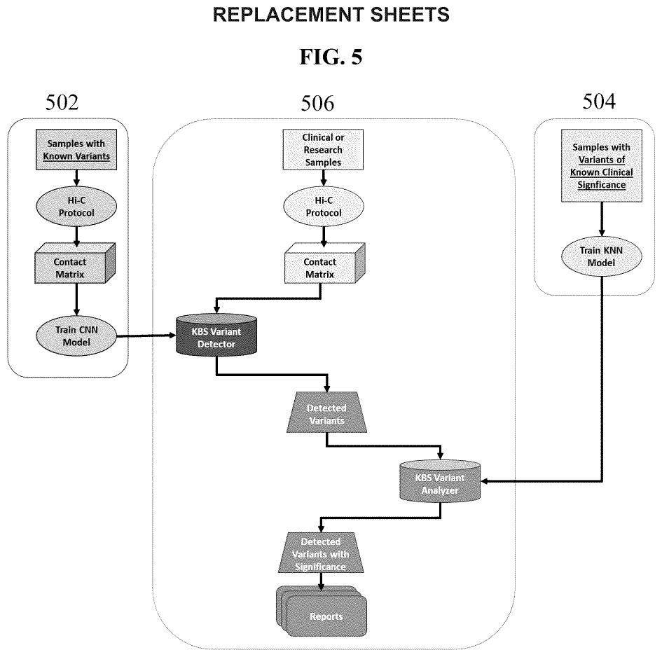

In another KBS implementation (FIG. 5), a set of deep learning models is created and used to identify any structural variant in an organism, and to assign possible actions, interpretations, or meanings to the variant based on known clinical or biological data. This implementation includes two machine learning models.

In this example, the first machine learning model is a convolutional neural network (CNN) which receives as input a contact matrix. This matrix may be averaged to a resolution such that feeding the matrix into a CNN would be computationally feasible (e.g., each cell in the matrix represents 1,000,000 base pairs), or a continuously scalable data structure (such as the KD-tree data structure described for the first major application). The first machine learning model detects regions of the contact matrix which appear to contain a structural variant, expressed as a bounding box in genomic coordinates, and also predicts a label for the variant (such as balanced or unbalanced translocation, inversion, insertion, deletion, repeat expansion). Alternatively the label may be a description of the variant that does qualitatively predict of the type of variant per se, but is input into the second machine learning model.

A CNN usable for this application can be defined with the following code in Python. This code is implemented in Keras with a TensorFlow backend as a custom CNN class. The function full_model(self, input_shape=(1000, 1000, 3), classes=5, verbose=False) constructs the full ResNet50 model. It takes the argument input_shape ((int, int, int)) which the shape of the images of the dataset. There must be 2 ints in a tuple (or list). It also takes the argument classes (int), which is number of classes and defaults to 1. It returns Keras.models.Model, which is the configured ResNet50 model. X_input defines the input as a tensor with shape input_shape. It then proceeds in 5 stages, shown below. The output layer makes individual layers and then concatenates them, allowing for the use of different activations in the output layer. Labels for the output layer are contains_event,

```
global_variant_start, global_variant_end, insertion_point and is_translocation.
print ‘Creating ResNet50 model with shape’, input_shape, ‘and’, classes, ‘classes. . .’
sys.stdout.flush( )
filters_1 = 32
filters_2 = [32, 32, 128]
filters_3 = [64, 64, 256]
filters_4 = [128, 12, 512]
filters_5 = [256, 256, 1024]
X_input = Input(input_shape)
X = ZeroPadding2D((2, 2,))(X_input)
# Stage 1
  X = Conv2D(32, (3, 3), strides = (1, 1), name = ‘conv1’,
    Kernel_initializer = glorot_uniform(seed=0))(X)
X = Conv2D(filters_1, (5, 5) strides = (3, 3)_, name = ‘conv1’,
X = BatchNormalization(axis = 3, name = ‘bn_conv1’)(X)
X = Activation(‘relu’)(X)
X = MaxPooling2D((3, 3), strides=(2, 2))(X)
X = Dropout(0.25)(X)
#Stage 2
X = self.convolutional_block(X, f = 3, filters = filters_2,
    stage = 2, block=’a’, s = 1)
X = self.identity_block(X, 3, filters_2, stage=2, block=’b’)
X = self.identity_block(X, 3, filters_2, stage=2, block=’c’)
X = Dropout(0.25)(X)
# Stage 4
X = self.convolutional_block(X, f = 3, filters = filters_4,
    stage = 4, block=’a’, s = 2)
X = self.identity_block(X, 3, filters_4, stage=4, block=’b’)
X = self.identity_block(X, 3, filters_4, stage=4, block=’c’)
X = self.identity_block(X, 3, filters_4, stage=4, block=’d’)
X = self.identity_block(X, 3, filters_4, stage=4, block=’e’)
X = self.identity_block(X, 3, filters_4, stage=4, block=’f’)
X = Dropout(0.25)(X)
# Stage 5
X = self.convolutional_block(X, f = 3, filters = filters_5,
    stage = 5, block=’a’, s = 2)
X = self.identity_block(X, 3, filters_5, stage=5, block=’b’)
X = self.identity_block(X, 3, filters_5, stage=5, block=’c’)
#AVGPOOL
X = AveragePooling2D(pool_size=(2,2), name=’avg_pool’)(X)
#output layer
X = Flatten( )(X)
  # X = Conv2D(5, 7, 7), name = ‘outcopy’,
    kernel_initializer = glorot_uniform(seed=0))(X)
  #X = Flatten( )(X)
  #X = Activation(‘sigmoid’)(X)
X = Dense(classes, activation= ‘linear’,
  Name= ‘fc’ + string(classes),
  kernel_initializer = glorot_uniform(seed=0))(X)
```

A CNN usable for this application can be compiled and trained in Python as described below. compile(self) compiles self.model so it is ready to run. train(self, X_train, Y_train, epochs=20, batch_size=32) trains self.model using X_train and Y_train, with mini-batches of size batch_size and for a number of training epochs equal to epochs. X_train and Y_train should be fully normalized and ready for training prior to calling this method. It takes the following arguments: X_train (np.vector[images]) is an input numpy vector of images to train with. Y_train (np.vector[np.vector[int]]) is the labels for the training images. epochs (int) is the number of training epochs to run, and batch.size (int) is

```
the size of minibatches to run.
print ‘Compiling ResNet50 model’
sys.stdout.flush( )
opt. = adam(lr=1e−6)
self.model.compile(optimizer=opt,#SGD(lr=1e−5),
  loss= ‘mse’,
  metrics=[‘accuracy’, ‘mse’, ‘mae’, float_accuracy(2), bin_acc])
print ‘ResNet50 model compiled’
sys.stdout.flush( )
print ‘Training ResNet50 model’
sys.stdout.flush( )
self.model.fit(X_train, Y_train, epochs = epochs, batch_size = batch_size)
print ‘ResNet50 training complete’
```

Both simulated and biological samples are used to train this machine learning model. First, the machine learning model is trained using contact matrices generated with a dataset containing all of the simulated samples, possibly in combination with a minority of data from biological samples. The contact matrices are fed into training both at full genome-wide scale, as well as zoomed in to portions of the matrix at a variety of resolutions.

Next, transfer learning is performed by clearing edge weights in the final several layers of the network, and the network is re-trained using the same methods but with data entirely from biological sources. This transfer learning step helps reduce the amount of genuine biological data required to train the model, which is important and advantageous to the overall design because obtaining detailed data about the tens of thousands or more actual cancer samples would be expensive (at least approximately $20 Million in sequencing costs alone), time consuming, and perhaps even impossible.

Once the machine learning model has obtained a set of regions which it has detected a variant at full genome scale, a complementary subroutine generates a contact map which zooms in on the portion of the contact matrix in which the variants were detected by generating a new submatrix at a finer resolution. For contact matrixes which include averaged data, this process generates submatrices which represent averages of smaller regions (e.g., a cell represents the average of 100,000 bp instead of 1,000,000 bp). For a continuously scaled contact matrix such as that represented by a KD-tree, the subroutine will zoom in by choosing the zoom factor for each region of interest on a continuous scale. The machine learning model runs again on these submatrices to refine the estimates for the bounding box, and correct the variant label if needed. This process is repeated recursively until satisfactory precision is obtained, enabling the high resolution of the Hi-C data to be leveraged without requiring a massive CNN. For example, this recursive process enables resolution of 1,000 bp or even finer on the human genome with a network containing a 300×300 input matrix by starting with each cell in the matrix representing 10,000,000 bp and recursively generating finer and finer submatrices until each cell in the matrix represented 1,000 bp. Conversely, without the recursive steps, a 30,000×30,000 input matrix would be needed for 1,000 bp resolution on the human genome. This represents a 10,000-fold increase in the number of input nodes required and greatly increases complexity deeper in the network, certainly making it extremely costly and possibly moving it into the realm of computational impossibility at current technological levels.

Once the first machine learning model has detected and labeled variants, a second machine learning model is used to relate the variants to known clinical or biological information. The second machine learning model is a k-nearest neighbors (KNN) model which associates the bounding boxes of specific variants, expressed in genomic coordinates, with curated clinical or biological data associated with the variant. This data is essentially similar to the data used in the Example 1, but expressed in genomic coordinates instead of genomic bands, and is not restricted to human samples. The second machine learning model is trained using contact matrixes from biological sources only, with the data labeled with known clinical or biological information such as specific diagnoses, patient outcomes, metabolic effect, associated drug targets/responses, and other actionable or relevant data.

After each machine learning model has been run on a sample, the results will be summarized in a report, such as PDF booklet, that will be returned the user. Importantly, the data and visualizations in the report will include information similar to that in a standard karyotype or FISH report that genetic counselors and clinicians typically see, even though they were not generated with those methods.

The steps below summarize the procedure for this example:
1. Map the Hi-C data to the organism's draft or reference genome (using BWA-mem).
2. Filter out low-quality alignment data (<MQ 20).
3. Transform the Hi-C genomic positions into a contact map.
4. Use CNN machine learning model to detect and label variants.
5. Repeat 3 and 4 until desired resolution is obtained, or no further improvement can be made.
6. Label each variant with relevant clinical or biological data using the second machine learning model.
7. Generate reports.

### Example 3: Detecting and Annotating all Structural Variants in an Organism Using an Edge Detection Algorithm

This is a multi-faceted approach that represents Hi-C link density between a pair of chromosomes as pixels in an image, then uses a series of image processing techniques and novel algorithms to identify translocation bounding boxes and the point of insertion. Pre-processing steps including global normalization, global thresholding, and per image de-noising are applied to the image, and then three edge/corner detection algorithms/modules (Harris corner method, Roberts cross, Hough transform) are used to identify large changes in the signal intensity gradient and convert those signals to bounding boxes (structural variant calls). Additional filters are applied to remove false positives, including a novel recursive algorithm for eliminating spurious detections close to the diagonal of intra-contig images.

False positive filtering techniques are non-trivial and are paramount to accuracy. Diagonal Path Finder (DPF), described below, is a false positive reducing algorithm used in this approach. Diagonal Path Finder is implemented in Python. This algorithm is used to determine whether or not a possible translocation is interchromosomal. Diagonal Path Finder works by walking up all possible Hi-C gradient paths. If no path reaches the main diagonal of the contact matrix, the translocation is interchromosomal. Given a row r and a column c of an upper triangular matrix “mat” of Hi-C data, “has_path_to_diag” determines whether or not here is a path to the diagonal that consists solely of cells with intensity >=mat[r, c]. The function has_path_to_diag (mat, r, c, val=None, exclude=None) has the parameters: mat (np.array): a 2-D array of intensity values; r (int): row index of the starting point; c (int): column index of the starting point; val (float): intensity of the starting point; exclude (set((int, int))): the set of (row, column) tuples that have been explored. The function returns: has_path (bool) which indicates whether or not there is a path to the diagonal; and exclude set ((int, int)), which is the set of (row, column) tuples that have been explored.

```
  if r>c:
    raise ValueError(‘Row must be <= column. Instead row = { } and col =
    { }’
.format(r, c)) if exclude is None:
    exclude = set( )
  if val is None:
    val = mat[r, c]
  if r = = c:
    return True, exclude
  exclude.add((r, c))
  has_path = False
  for (row, col) in [r (r+1, c−1), (r+1, c), (r, c−1)]:
    if (mat[row, col] >= val) and (row <= col) and (not has_path) and \
      ((row, col) not in exclude);
        has_path, exclude = has_path_to_diag(mat, row, col, val=val,
exclude=exclude)
  return has_path, exclude
```

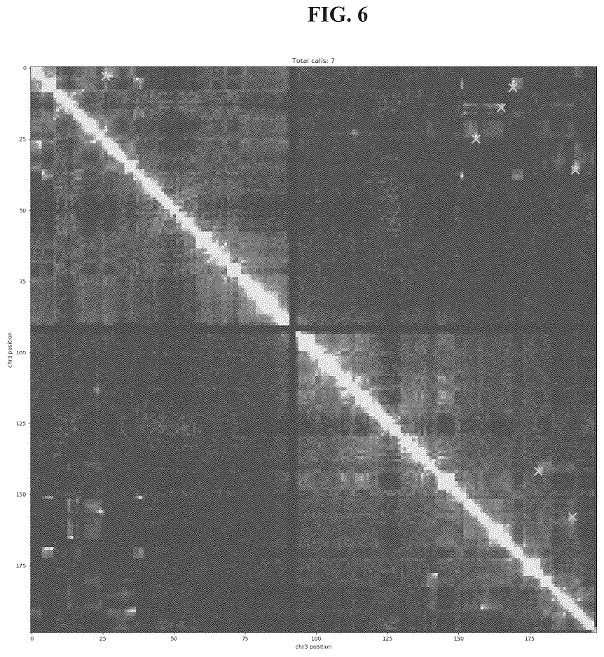

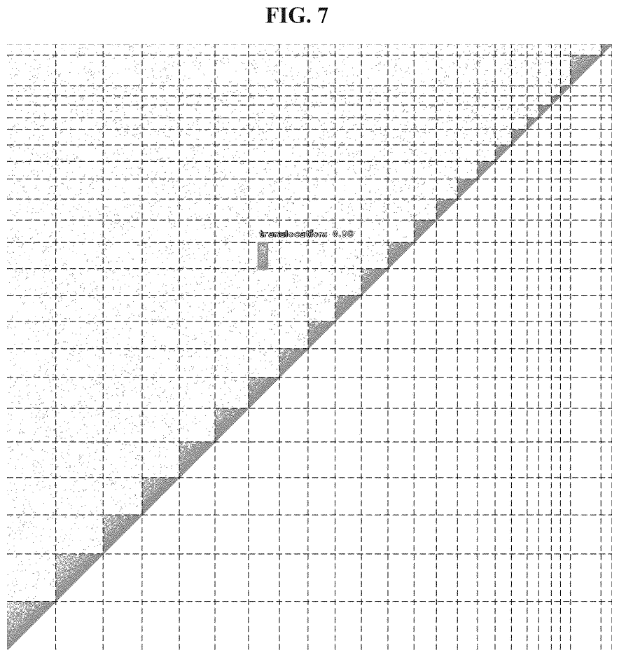

Finally, we output a set of translocation calls in the standard Variant Call Format (VCF). The prototype code is already producing reliable calls on clinical data. The results of the edge detection algorithm(s) can be seen in FIG. 7 where seven novel de novo large-scale intra chromosomal events have been identified. An example image of a contact matrix showing chromosome 3 from a cancer sample is shown in FIG. 6. The marked corners correspond to structural variants on the chromosome.

The steps performed in this embodiment can be summarized as follows:
1) Store interactions in a compressed, sparse matrix representation (40 Kbp bins)
2) Fit a set of weights that force row and column sums to be close to zero (ignoring bins within 100 Kbp of diagonal) and use them to calculate balanced interaction density for each bin
3) Calculate global thresholds using balanced interaction density
   a) Median for each diagonal of cis-chromosome pairs
   b) Use median balanced interaction density Y of bins at X bp from diagonal as minimum threshold for corners (for example 4 Mbp).
4) For each sub region of the matrix (chromosome comparisons)
   a) Clip balanced density values to 2*Y (prevents diagonal from washing out signal)
   b) Denoise submatrix (Use bilateral method to preserve edges)
   c) Use resulting pixel intensity values (Z)
   d) Detect corners (Harris corner method or Roberts cross*Z)
      e) Filter false positives
      f) Non-max suppression (removes cases with multiple calls for a single peak)
      g) Diagonal climb (removes calls due to spurious, strong edges near diagonal while preserving inversions)
      h) Neighbor threshold (removes calls from single hot pixel)
5) Reconstruct translocation call in VCF format
6) Summarize events in PDF report.

### Example 4: Simulating Chromosomal Structural Variants in Chromosome Conformational Capture Data

Given the high costs of sequence large numbers of samples, it can be advantageous to train machine learning models used in the methods disclosed herein using simulated Hi-C. Described below is a method, in Python, which initializes a class capable of simulating structural variations, such as cancer mutations and balanced translocations, unbalanced translocations, insertions, and deletions, and generating simulated Hi-C data based on these simulated structural variations.

Class HiCSimulator simulates HiC data. It has the properties: fai (str): the fai that was used to initialize the simulator; gv (list): a genome vector; chrom_bin_lengths (str: int): the length of each chromosome, in bins; bin_size (int): the size of the bins to make; reads (int): the number of intracontig reads to simulate; background_reads (int): the number of intercontig reads to simulate; max_coordinate (int): the max coordinate in the assembly, for converting bp to pixels simulate which defaults to 0.1% of reads; chrom_bounds (dict[tuple[int, int]): global start and end coordinates for each chromosome. The class HiCSimulator is initialized as follows:

```
def_init_(self, fai, bin_size, reads, background_reads = None):
  random.seed( )
  self.fai = fai
  self.bin_size = bin.size
  self.reads = reads
    self.background.reads = background_reads if background_reads is not
    None else
  int)(0.001*reads)
  self.max_coordinate = 0
  self.chrom_bounds = dict( )
  self.chrom_bin_bounds = dict( )
  self.gv = [ ]
  offset = −1 * bin_size;
  offset_count = −1
  chr_dest = ‘a’
  with open(fai) as tsv:
      for line in csv.read(tsv, delimiter=”\t”):
      start = −1 * bin_size;
      end = −1 * bin_size;
      if int(line[1]) + int(line[2]) > self.max_coordinate:
        self.max_coordinate = int(line[1]) + int(line[2])
      self.chrom_bounds[line[0]] = (int(line[2]), int(line[1])−int(line[2]))
      self.chrom_bin_bounds[line[0]] = [None, None]
  while (end < int(line[1])):
      start += bin_size
      end = start + bin size
      if end > int(line[1]):
        end = int(line[1])
      offset += end − start
      offset_count += 1
          if self.chrom_bin_bounds[line[0]][0] is None or
  self.chrom_bin_bounds[line[0]][0] > offset_count:
        self.chrom_bin_bounds[line[0]][0] = offset_count
      if self.chrom_bin_bounds[line[0]][1] is None or
  self.chrom_bin_bounds[line[0]][1] < offset count:
        self.chrom_bin_bounds[line[0]][1] = offset_count
```

| | | |
|---|---|---|
| bin_datum = { | ‘chr’ | : line[0], |
| | ‘beg’ | : start, |
| | ‘end’ | : end, |
| | ‘width’ | : end − start, |
| | ‘offset’ | : offset #genomic offset |
| | ‘cnf’ | : 1, #copy number float |
| | ‘offset_count’ | : 0, |
| | ‘offset_per’ | : 0, |
| | ‘event’ | : “none” |
| | } | |

```
      self,gv.append(bin_datum)
self.chrom_bin_lengths = collections.defaultdict(lambda: 0)
for bin in self.gv:
  self.chrom_bin_lengths[bin[‘chr’]] += 1
```

The Customer HiCSimulator class is used to simulate structural variations such as cancer mutations, and simulates Hi-C data based on these simulated structural variations following a statistical model of the biochemical characteristics of the Hi-C protocol in Python.

```
def make_heatmap_data(self, sv_bins_length, heatmap_data_file, label_file, verbose=False,
make_null_example=False, heatmap_id=”, img_height=1000, img_width=1000,
img_depth=3):
  if verbose:
      print ‘Simulating data from’, self.fai
      print ‘bin_size =’, self.bin_size
      print ‘reads =’, self.reads
      print ‘background_reads =’, self.background_reads
      print ‘sv_bins_length =’, self.sv_bins_length
      print ‘heatmap_data_file =’, heatmap_data_file
      print ‘label_file =’, label_file
      print ‘make_null_example =’, make_null_example
      print ‘heatmap_id =’, heatmap_id
      print ‘img_height =’, img_height
      print ‘img_width =’, img_width
      print ‘img_depth =’, img_depth
      print ‘verbose =’, verbose
  chr_dest = ‘α’
  chr_src = ‘α’
  gv = deepcopy(self.gv)
  while(chr_dest = = chr_src):
      #the source piece must be sv_bins_length
      r_dest = self.find_within_chr(gv, sv_bins_length)
      #the destination can be any point
      r_dest = self.find_within_chr(gv, 1)
      chr_dest = gv[r_dest][‘chr’]
      chr_src = gv[r_src][‘chr’]
  if(r_dest < 0 or r_src< 0):
      raise ValueError (‘failed to find insertion point’)
  src_start = r_src
  src_end = r_src+sv_bins_length
  if gv[src_start][‘chr’] != gv[scr_end][‘chr’]
      raise ValueError(‘Source chromosomes don\'t match! {0}:{1}, {2}:{3}’\
            .format(src_start, gv[src_start][‘chr’], src_end,
gv[src_end][‘chr’]))
  if not make_null_example:
      for i in range(src_start, src_end):
        gv[i][‘cnf’] += 1
        gv[i][‘event’] = ‘t’
      for i in range(0, length(gv)):
        if (gv[i][‘chr’] = = chr_dest):
          gv[i][‘event] = ‘t’
  event_type = ‘null’ if make_null_example else gv[r_dest][‘event’]
  variant_start = gv[src_start][‘beg’]
  variant_end = gv[src_end][‘end’]
  dest_start = gv[r_dest][‘beg’]
  dest_end = gv[r_dest][‘end’]
  event_width = variant_end − variant_start
  event_code = ‘{0}({1}[{2}-{3}], {4}[{5}])’.format(envent_type,
                                      chr_src,
                                      variant_start,
                                      variant_end,
                                      chr_dest,
                                      dest_start)
label = label(labeled_file=heatmap_id, img_height=img_height, img_width=img_width,
          img_depth=img_depth, source= ‘Simulated data’)
if event_type != ‘null’;
  #label normalizes to pixel space
  if r_src >= r_dest:
    label.add_labeled_object(‘translocation’,
      int(round(img_width *
float(self.chrom_bin_bounds[chr_dest][0])/len(self.gv))),
      int(round(img+width *
float(self.chrom_bin_bounds[chr_dest][1])/len(self.gv))),
      int(round(img_height * (1.0 − float(src_end)/len(self.gv)))),
      int(round(img_height * (1.0 − float(src_start)/len(self.gv))))
  else:
    label.add_labeled_object(‘translocation’,
      int(round(img_width * float(src_start)/len(self.gov))),
      int(round(img_width * float(src_send)/len(self.gov))),
        int(round(img_height * (1.0 −
float(self.chrom_bin_bounds[chr_dest][1])/len(self.gv)))),
      int(round(img_height * (1.0 −
float(self.chrom+bin_bounds[chr_dest][0])/len(self.gv)))))
# writing the labels clears out the current contents of the files
with open(heatmap_data_file, ‘w’) as f:
  f.write(event_code+ ‘\n’)
label.write_label_to_xml_file(label_file)
if verbose:
  print ‘Variant moves {0}-{1}({2}kbp, {3} bins) on {4} to {5} on {6}’.format(
                    variant_start, variant_end,
                    (variant_end-variant_start)/1000,
                    (variant_end-variant_start)/self.bin_size,
                    Chr_src, gv[r_dest][‘beg’], chr_dest)
  print ‘event code:’, event_code
  print ‘Label:’, label
  print ‘Bins:’, float(src_start)/len(self.gv) * self.max_coordinate/1e6,\
          float(src_end/len(self.gv) * self.max_coordinate/1e6,\
          self. chrom_bin_bounds[chr_dest][0],\
          self. chrom_bin_bounds[chr_dest][1],
gv_len = len(gv)
offc = 0
for k in range (0, len(gv)):
```

| | |
|---|---|
| gv[k][‘offset_count’] | = gv_len − offc |
| gv[k][‘offset_per’] | = gv_len − offc |

```
  offc += 1
binned_data =collections.defaultdict(lambda: 0)
read_pairs = 0
tmp_bin = 0
if verbose:
  print ‘Writing’, self.reads, ‘intrachromosomal reads. . .’
while(read_pairs < self.reads):
  r_bin_one =int(random.uniform(0-, gv_len))
  #r_bin_two = int(random.uniform(r_bin_one, gv_len))
  #r_bin_one = 950
  #r_bin_two = int(random.uniform(0,, gv[r_bin_one][‘offset_count’]))
  r_bin_two = int(random.uniform(r_bin_one, gv[r_bin_one][‘offset_count’]))
  if(gv[r_bin_one][‘chr’] != gv[r_bin_two]p’chr’]:
        if (gv[r_bin_one][‘event’]!= ‘t’ or gv[r_bin_two][‘event’]!= ‘t’):
  gv[r_bin_one][‘offset_count’] = r_bin_two
  if(r_bin_two < r_bin_one):
    tmp_bin = r_bin_two
    r_bin_two = r_bin_one
    r_bin_one = tmp_bin
  read_pairs += 1
  binned_data[’{0}:{1}’.format(r_bin_one, r_bin_two)] +30=1
read_pairs =0
if verbose:
  print ‘Writing’, self.background_reads, ‘background reads. . .’
while(read_pairs < self.background_reads);
  r_bin_one = int(random.uniform(0, len(gv)))
  r_bin_two = int(random.uniform(0, len(gv)))
  if(r_bin_two < r_bin_one):
      tmp_bin = r_bin_two
      r_bin_two = r_bin_one
      r_bin_one = tmp_bin
  read_pairs += 1
  binned_dta[‘{0}:{1}’. format(r_bin_one, r_bin_two)]+=1
with open(heatmap_data_file, ‘a’) as f:
  for key in binned_data:
      kv = key.split(‘:’)
      if(gv[int(kv [0])][‘offset’] < gv[ing(kv[1])]{‘offset’]):
        f.write(‘{0} {1} {2} {3} {4}\n’.format(gv[int(kv[0])][‘offset’],
                                  gv[int(kv[1])][‘offset’],
                                  binned_dta[key],
                                  gv[int(kv[0])][‘chr’],
                                  gv[int(kv[1])][‘chr’]))
return label
```

### Example 5: Comparing Karyotype by Sequencing (KBS) Methods with Other Methods for Detecting Chromosomal Structural Variants

Using data from a leukemia sample, the deep-learning-based Karyotype by Sequencing (KBS) method was compared to three other current methods for detecting structural variants in Hi-C datasets. These included the following:
hic_breakfinder (described in Dixon, Jesse R et al. “Integrative detection and analysis of structural variation in cancer genomes.” Nature genetics vol. 50,10 (2018): 1388-1398. doi: 10.1038/s41588-018-0195-8),
CNVnator (described in Abyzov, Alexej, et al. “CNVnator: an approach to discover, genotype, and characterize typical and atypical CNVs from family and population genome sequencing.” Genome research 21.6 (2011): 974-984), and
HiNT (described in Wang, Su, et al. “HINT: a computational method for detecting copy number variations and translocations from Hi-C data.” biorxiv (2019): 657080).

These tools all use human-defined algorithms for recognizing signatures of structural variants, as opposed to the deep-learning-based KBS approach. Hic_breakfinder aggregates and filters the results of 3 different tools: DELLY, Lumpy, and Control-FREEC. DELLY uses a dynamic programming approach on alignment and kmer data. Lumpy uses alignment to identify adjacent base pairs in sequence data which are not adjacent in the reference genome and calculates a probability distribution for the base pairs reflecting a real difference relative to the reference. Control-FREEC estimates copy number and is used to refine the calls made by DELLY or Lumpy, and tries to identify deletions. CNVnator looks for changes in coverage to identify changes in copy number variation, which is the standard approach. CNVator refines the standard approach with a partitioning scheme that lets it deal with noise/variation in coverage, and correct for GC content. HiNT detects copy number variation in a method similar to CNVnator, except it attempts to correct for GC content, mappability, and restriction fragment length. To find translocations, it identifies possible SV regions by looking at 1-dimensional Hi-C data, then examines the reads that align to those regions. In contrast to these methods, KBS learns what different kinds of variants look like, as opposed to defining a model of what the data look like in the absence of structural variants. KBS then computes a probability that there is a variant in a given dataset.

Karyotyping and FISH analyses were previously performed against this sample, providing a ground-truth for which variants are expected to be present in the sample. Table 5 below shows the variants detected using traditional cytogenetics, and how well they were detected by each Hi-C-based method. In table 5, “count” refers to counting true and false positives, missing an event of any size is of equal weight. “bp” refers to weighting those calls by the size of the event, so missing a 1 megabase call is 1,000 times “worse’ than missing a 1 kilobase call.

TABLE 5
Comparison of KBS and other methods

| | Event Size | | | | |
|---|---|---|---|---|---|
| Event | (bp) | CNVnator | hic_breakfinder | HiNT | KBS |
| t(1q21; 17p13) | 22,700,000 | 0 | 1 | 0 | 1 |
| t(2; 9; 4)(p23; p23; q25) | 124,400,000 | 0 | 1 | 1 | 1 |
| del(4)(q27q31) | 14,600,000 | 1 | 0 | 0 | 0 |
| der(12)t(12; 17)(p13; q11.2) | 21,200,000 | 0 | 1 | 0 | 1 |
| trisonny chr18 | 80,373,285 | 1 | 0 | 0 | 1 |
| add(4)(q35) | 7,914,555 | 0 | 0 | 0 | 0 |
| del(4)(q11.2q25) | 8,300,000 | 1 | 0 | 0 | 1 |
| CDK2N2A x0 (c.hr9) | 26,871 | 1 | 0 | 0 | 0 |
| True positive | | 4 | 3 | 1 | 5 |
| False negative | | 4 | 5 | 7 | 3 |
| False positive | | 33 | 17 | 0 | 3 |
| Sensitivity (count) | | 50% | 38% | 13% | 63% |
| Sensitivity (bp-based) | | 37% | 60% | 45% | 92% |
| False Discovery Rate | | 89% | 85% | 0% | 37.5% |

The data in table 5 shows how KBS, CNVator, hic_breakfinder and HiNT performed against a real, karyotyped data set that also had 1 FISH test performed. Generally CNVator, hic_breakfinder and HiNT methods are less comprehensive than karyotyping, and have coarser resolution than FISH. Furthermore, Hic_breakfinder struggles to detect deletions, insertions, or aneuploidies. CNVnator cannot detect translocations. HINT claims to be able to do both, but the method is lacking in actual capabilities as can be seen from Table 5. Further, only KBS is a learning model, meaning its performance over time will improve as it has access to more data. The results in Table 5 were generated using a KBS system trained with 10,000 simulated Hi-C datasets only.

The KBS method showed significantly better sensitivity to detecting structural variants, particularly when weighting each variant based on the number of base pairs it affects. Additionally, its false discovery rate is significantly better than two of the other methods, and the only other approach with a better false discovery rate had very poor sensitivity, only detecting one of eight true events as well.

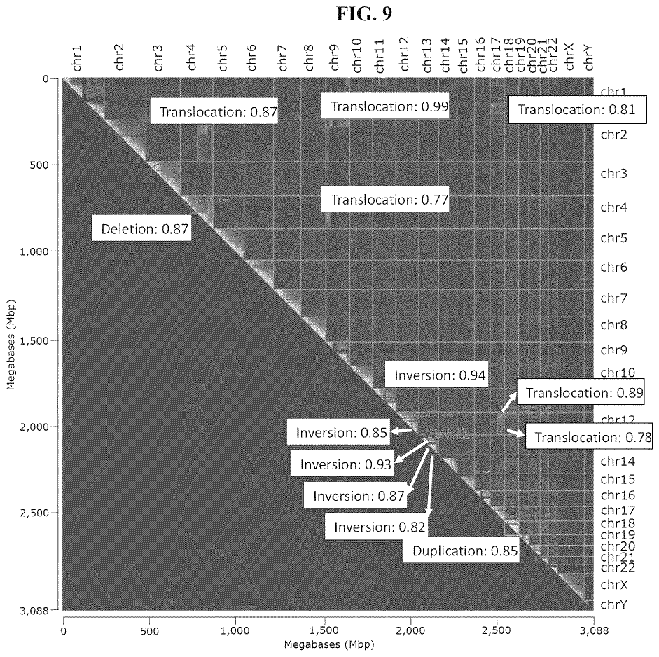

FIG. 9 shows the events detected by KBS in the leukemia sample. The three red boxes along the top edge of FIG. 9 are the three false positives listed in Table 5, which seem to be related to a common biological feature of chromosome 1. Since KBS is deep-learning-based, training the system with more data will likely to reduce false discovery rate by learning as KBS is trained to understand which patterns are within normal biological variation.

Table 6 below compares the capabilities of the KBS system to comparable in-market cytogenetic methods. KBS methods represent a significant improvement over the current tests available in clinical settings. These methods include conventional karyotyping, FISH, and chromosomal microarray (CMA).

TABLE 6
KBS versus current cytogenetic methods

| | Karyo- | | | Hi-C |
|---|---|---|---|---|
| Aberration | typing | FISH | CMA | KBS |
| Genome-wide detection | Yes | No | Yes | Yes |
| Unbalanced Chromosomal | Yes | Yes | Yes | Yes |
| alterations | | | | |
| (deletion/duplication/ | | | | |
| amplication) | | | | |
| Balanced rearrangements | Yes | Yes | No | Yes |
| (translocation/ | | | | |
| inversion/insertion) | | | | |
| Complex rearrangement | Some | No | No | Yes |
| Chromothripsis (cth) | No | No | Yes | Yes |
| Resolution (bp) | 10⁶ | 10⁵ | 10⁴-10⁵ | 10³-10⁴ |
| Turn around time | 1-3 weeks | 1-3 weeks | 1-3 weeks | 2-7 days |
| Diseases/conditions/ | 10³ | 10¹ | 10¹ | 10⁴ |
| markers per test | | | | |
| Cost | $1000 | $1000 | $1000 | $1000 |

### Example 6: Convolutional Neural Network (CNN) Model Design

Two common CNN architectures, resnet-50 and RetinaNet, provided a suitable starting point for the detection of structural variants in Hi-C matrixes.

Using a small simulated Hi-C dataset in a modified resnet-50 network, 96.5% accuracy was achieved in detecting the presence of unbalanced translocations in a sample, with a loss of 3.29%. The bounding box of such translocations was identified with an accuracy of 59.5% and a loss of 3.58%.

Testing the same data in RetinaNet, an average precision in excess of 95% was achieved for detecting the location simulated events over 1 Mbp. These results demonstrate that performance at least comparable to karyotyping is achievable with this approach, despite only using a small amount of simulated data and a relatively unmodified CNN. With additional training data, customization of the CNN model (including testing other network approaches such as that illustrated by yolo-v3; Redmon, J. and Farhadi, A., 2018. Yolov3: An incremental improvement. arXiv preprint arXiv: 1804.02767), and identification of optimal hyperparameters, model performance will be improved. Due to the nature of identifying events with CNNs, a variant-class label and confidence score for each call made by the CNN can be used to classify events and filter out low-confidence events to improve sensitivity and specificity.

### Example 7: Training Machine Learning Models

Obtaining sufficient high-quality labeled data is critical to the implementation of a deep learning system, which can be an expensive and challenging problem in genomics. To address these issues, the CNN will be trained using a mixture of simulated Hi-C data and real-world Hi-C data in a two-stage transfer learning process.

First, simulated positive samples will be generated by randomly creating structural variants (SVs) and copy number variants (CNVs) in the human reference genome, and then simulating Hi-C data from these SVs and CNVs. Because the variations in these samples will be generated computationally, it will also be possible to provide exact labels for them detailing what variations have been represented within the simulated Hi-C data. Additionally a set of simulated data will be generated to provide negative controls to the CNN.

After training the CNN on a large body (several million or more if necessary) of simulated samples, transfer learning will be performed by clearing the weights in the final one to two layers of the CNN and re-training the weights on only those layers using real Hi-C data from a smaller number of both healthy and tumor tissue samples (~500). This approach allows for the use of relatively cheap simulated data to train the network to detect basic features in Hi-C datasets, while using more expensive real-world data to train it on how to extrapolate genuine SV and CNV calls from those features.

### Example 8: Normalizing Hi-C Data Relative to Healthy Cells and Identifying Fine-Scale Variants

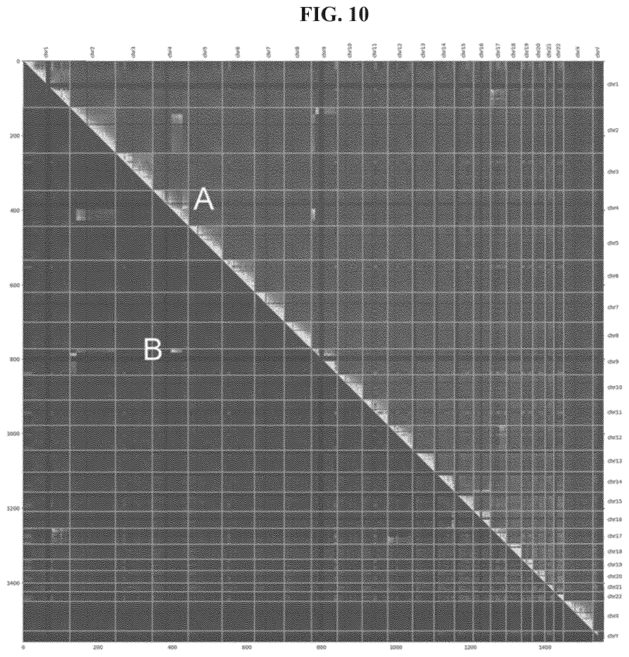

Raw Hi-C data are useful for identification of fine-scale variations in chromatin structure as well as CNVs such as deletions and duplications. However, natural chromatin structures such as topologically associating domains (TADs) and A/B compartments can create false positives, and as such methods which analyze Hi-C data often include normalization procedures to exclude such effects. The symmetric nature of Hi-C datasets to allows the generation a matrix reflecting both raw and normalized versions of the Hi-C data, where the normalized version is generated by dividing the raw Hi-C matrix by a background model generated from healthy tissue (FIG. 10).

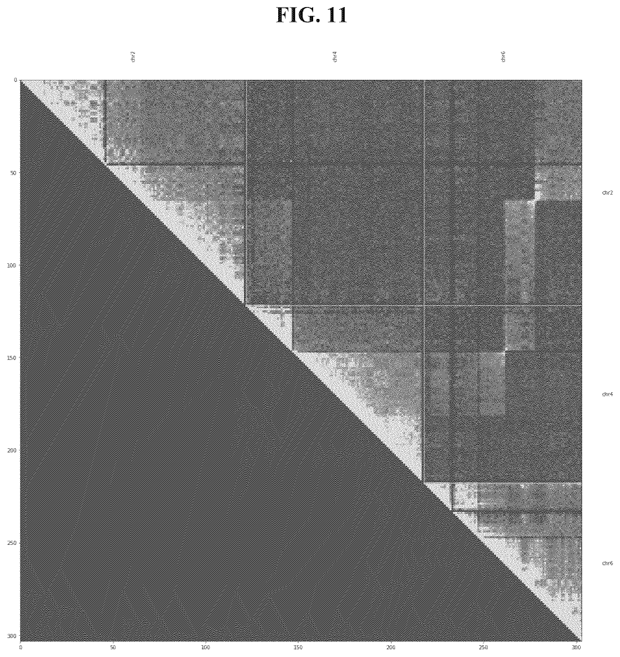

To provide the ability to achieve resolution of variants at least as fine as FISH (10⁵ bp) without requiring the CNN to have millions of input nodes, the Hi-C data will be generated at multiple scales and analyze it recursively. Initially, the matrix will be generated and examined at the genome-wide level by breaking it into several hundred to several thousand bins (exact initial bin size is a tradeoff between initial resolution and performance, which will be determined through experimentation). Bounding boxes for possible SVs and CNVs will be identified in the initial matrix by the CNN. For each such bounding box an additional matrix will be generated which zooms into the coordinates of bounding box at finer resolution, with the specific resolution determined by the size of the bounding box and the number of nodes in the input layer of the CNN. Each such matrix will be and passed back through the CNN to generate one or more refined bounding box coordinates. This process will be repeated recursively until desired resolution (10 kb) is obtained, or the bounding box cannot be refined further. In this manner, zooming in enables fine-scale analysis of complex structural variants that exceed the capabilities of other analysis methods (FIG. 11). By ensuring training data includes labeled examples of complex variants, the CNN will have the opportunity to learn how to recognize such events from their Hi-C patterns.

While illustrative embodiments have been illustrated and described, it will be appreciated that various changes can be made therein without departing from the spirit and scope of the invention.

1. A computer-implemented method of improving computational efficiency of processing high-resolution chromatin interaction information to generate karyotypic data, the method comprising:
receiving, by a computing system, a contact matrix based on a biological sample;
generating, by the computing system, a first contact matrix at a first resolution based on the received contact matrix, wherein the first contact matrix has a size that corresponds to an input dimension of a first machine learning model;
processing, by the computing system, the first contact matrix using the first machine learning model to detect at least one region of the first contact matrix that appears to contain a chromosomal structural variant, wherein output of the first machine learning model includes a bounding box and a variant label for each region that appears to contain a chromosomal structural variant;
for each region of the at least one region that appears to contain a chromosomal structural variant:
generating, by the computing system, a second contact matrix at a second resolution based on a sub-portion of the received contact matrix, wherein the second contact matrix has a size that corresponds to the input dimension of the first machine learning model, wherein the sub-portion of the received contact matrix includes the bounding box of the region, and wherein the second resolution is finer than the first resolution; and
processing, by the computing system, the second contact matrix using the first machine learning model to generate an updated bounding box and an updated variant label for the region; and
processing, by the computing system, each updated bounding box and updated variant label using a second machine learning model to generate output that relates chromosomal structural variants detected in the biological sample to at least one of a known clinical significance or a known biological significance.

2. The computer-implemented method of claim 1, further comprising:
repeating generation of contact matrices at finer resolutions and processing using the first machine learning model to update the bounding boxes and the variant labels for each region until a predetermined precision is reached.

3. The computer-implemented method of claim 1, wherein generating the first contact matrix at the first resolution based on the received contact matrix includes averaging, by the computing system, the received contact matrix to the first resolution; and
wherein generating the second contact matrix at the second resolution based on a sub-portion of the received contact matrix includes averaging, by the computing system, the sub-portion of the received contact matrix to the second resolution.

4. The computer-implemented method of claim 1, further comprising converting, by the computing system, the received contact matrix into a continuously scalable data structure;
wherein generating the first contact matrix at the first resolution based on the received contact matrix includes extracting, by the computing system, the first contact matrix from the continuously scalable data structure at the first resolution; and
wherein generating the second contact matrix at the second resolution based on the received contact matrix includes extracting, by the computing system, the second contact matrix from the continuously scalable data structure at the second resolution.

5. The computer-implemented method of claim 4, wherein the continuously scalable data structure is a KD-tree.

6. The computer-implemented method of claim 1, further comprising:
generating, by the computing system, a report comprising karyotypic data for the biological sample, wherein the karyotypic data comprise:
identification of one or more chromosomal structural variants indicated by the updated bounding boxes and updated variant labels; and
annotation of the at least one of the known clinical significance or the known biological significance.

7. The method of claim 1, wherein the first machine learning model includes a convolutional neural network (CNN) trained by:
training the CNN using a first training dataset that includes contact matrices generated from simulated samples and, optionally, biological samples;
clearing edge weights in one or more final layers of the CNN; and
re-training the CNN using a second training dataset that includes contact matrices generated from biological samples and does not include contact matrices generated from simulated samples.

8. The method of claim 1, wherein the second machine learning model comprises a k-nearest neighbors model.

9. The method of claim 1, wherein the high-resolution chromatin interaction information is generated using a chromatin conformation analysis technique comprising one or more of chromatin conformation capture (3C), circularized chromatin conformation capture (4C), carbon copy chromosome conformation capture (5C), chromatin immunoprecipitation (ChIP), ChIP-Loop, Hi-C, combined 3C-ChIP-cloning (6C), Capture-C, Split-pool barcoding (SPLIT-seq), Nuclear Ligation Assay (NLA), Single-cell Hi-C (scHi-C), Combinatorial Single-cell Hi-C, Concatamer Ligation Assay (COLA), Cleavage Under Targets and Release Using Nuclease (CUT& RUN), in vitro proximity ligation (Chicago®), in situ proximity ligation (in situ Hi-C), proximity ligation followed by sequencing on an Oxford Nanopore machine (Pore-C), proximity ligation sequenced on a Pacific Biosciences machine (SMRT-C), DNase Hi-C, Micro-C, or Hybrid Capture Hi.C.

10. The method of claim 1, wherein the known biological significance or the known clinical significance includes one or more of biological information relating to one or more genes, a diagnosis, a patient outcome, a metabolic effect, a drug target, a drug response, or a course of treatment.

11. A non-transitory computer-readable medium having computer-executable instructions stored thereon that, in response to execution by one or more processors of a computing system, cause the computing system to perform actions for improving computational efficiency of processing high-resolution chromatin interaction information to generate karyotypic data, the actions comprising:
receiving, by the computing system, a contact matrix based on a biological sample;
generating, by the computing system, a first contact matrix at a first resolution based on the received contact matrix, wherein the first contact matrix has a size that corresponds to an input dimension of a first machine learning model;
processing, by the computing system, the first contact matrix using the first machine learning model to detect at least one region of the first contact matrix that appears to contain a chromosomal structural variant, wherein output of the first machine learning model includes a bounding box and a variant label for each region that appears to contain a chromosomal structural variant;
for each region of the at least one region that appears to contain a chromosomal structural variant:
generating, by the computing system, a second contact matrix at a second resolution based on a sub-portion of the received contact matrix, wherein the second contact matrix has a size that corresponds to the input dimension of the first machine learning model, wherein the sub-portion of the received contact matrix includes the bounding box of the region, and wherein the second resolution is finer than the first resolution; and
processing, by the computing system, the second contact matrix using the first machine learning model to generate an updated bounding box and an updated variant label for the region; and
processing, by the computing system, each updated bounding box and updated variant label using a second machine learning model to generate output that relates chromosomal structural variants detected in the biological sample to at least one of a known clinical significance or a known biological significance.

12. The non-transitory computer-readable medium of claim 11, wherein the actions further comprise:
repeating generation of contact matrices at finer resolutions and processing using the first machine learning model to update the bounding boxes and the variant labels for each region until a predetermined precision is reached.

13. The non-transitory computer-readable medium of claim 11, wherein generating the first contact matrix at the first resolution based on the received contact matrix includes averaging, by the computing system, the received contact matrix to the first resolution; and
wherein generating the second contact matrix at the second resolution based on a sub-portion of the received contact matrix includes averaging, by the computing system, the sub-portion of the received contact matrix to the second resolution.

14. The non-transitory computer-readable medium of claim 11, wherein the actions further comprise converting, by the computing system, the received contact matrix into a continuously scalable data structure;
wherein generating the first contact matrix at the first resolution based on the received contact matrix includes extracting, by the computing system, the first contact matrix from the continuously scalable data structure at the first resolution; and
wherein generating the second contact matrix at the second resolution based on the received contact matrix includes extracting, by the computing system, the second contact matrix from the continuously scalable data structure at the second resolution.

15. The non-transitory computer-readable medium of claim 14, wherein the continuously scalable data structure is a KD-tree.

16. The non-transitory computer-readable medium of claim 11, wherein the actions further comprise:
generating, by the computing system, a report comprising karyotypic data for the biological sample, wherein the karyotypic data comprise:
identification of one or more chromosomal structural variants indicated by the updated bounding boxes and updated variant labels; and
annotation of the at least one of the known clinical significance or the known biological significance.

17. The non-transitory computer-readable medium of claim 11, wherein the first machine learning model includes a convolutional neural network (CNN) trained by:
training the CNN using a first training dataset that includes contact matrices generated from simulated samples and, optionally, biological samples;
clearing edge weights in one or more final layers of the CNN; and
re-training the CNN using a second training dataset that includes contact matrices generated from biological samples and does not include contact matrices generated from simulated samples.

18. The non-transitory computer-readable medium of claim 11, wherein the second machine learning model comprises a k-nearest neighbors model.

19. The non-transitory computer-readable medium of claim 11, wherein the high-resolution chromatin interaction information is generated using a chromatin conformation analysis technique comprising one or more of chromatin conformation capture (3C), circularized chromatin conformation capture (4C), carbon copy chromosome conformation capture (5C), chromatin immunoprecipitation (ChIP), ChIP-Loop, Hi-C, combined 3C-ChIP-cloning (6C), Capture-C, Split-pool barcoding (SPLIT-seq), Nuclear Ligation Assay (NLA), Single-cell Hi-C (scHi-C), Combinatorial Single-cell Hi-C, Concatamer Ligation Assay (COLA), Cleavage Under Targets and Release Using Nuclease (CUT& RUN), in vitro proximity ligation (Chicago®), in situ proximity ligation (in situ Hi-C), proximity ligation followed by sequencing on an Oxford Nanopore machine (Pore-C), proximity ligation sequenced on a Pacific Biosciences machine (SMRT-C), DNase Hi-C, Micro-C, or Hybrid Capture Hi-C.

20. The non-transitory computer-readable medium of claim 11, wherein the known biological significance or the known clinical significance includes one or more of biological information relating to one or more genes, a diagnosis, a patient outcome, a metabolic effect, a drug target, a drug response, or a course of treatment.
